(19) United States
(12) Patent Application Publication
(10) Pub. No.: US 2024/0252428 A1
(43) Pub. Date: Aug. 1, 2024

(54) METHODS OF PROVIDING BIRTH CONTROL

(71) Applicant: THE POPULATION COUNCIL, INC., New York, NY (US)

(72) Inventors: George William CREASY, II, Glen Gardner, NJ (US); Ruth Beverly MERKATZ, Rye, NY (US); Marlena PLAGIANOS, Bardonia, NY (US); Regine SITRUK-WARE, Paris (FR); Bruce VARIANO, New York, NY (US)

(21) Appl. No.: 18/559,268

(22) PCT Filed: May 6, 2022
(86) PCT No.: PCT/US2022/028058

§ 371 Date: Nov. 6, 2023

Related U.S. Application Data
(60) Provisional application No. 63/185,809, filed on May 7, 2021.

(51) Int. Cl. A61K 9/00; A61K 31/565; A61K 31/573; A61P 15/18

(52) U.S. Cl. CPC A61K 9/0036; A61K 31/565; A61K 31/573; A61P 15/18

(57) ABSTRACT

The present disclosure relates to a vaginal system that prevents pregnancy comprised of segesterone acetate and ethinyl estradiol and is suitable for four quarterly product-use cycles or for a 365-day product-use cycle.

### FIELD

The present disclosure relates to methods of providing birth control using a vaginal system comprising a progestin, such as segesterone acetate, and an estrogen, such as ethinyl estradiol, that is suitable for four quarterly product-use cycles or one annual product-use cycle.

### BACKGROUND

The use of oral contraception is widespread in the female population. But the need to remember a daily pill and the inconvenience of having to obtain frequent refills can reduce compliance, jeopardizing its effectiveness.

The use of subcutaneous upper arm implants and intrauterine devices (IUDs) as a means of administering contraception is often seen as a way of overcoming these drawbacks as they remain effective for more than one year. These devices, however, have their own disadvantages as insertion and removal of implants and IUDs require a medical professional, such as a doctor, nurse, or physician's assistant.

Intravaginal rings are annularly shaped articles containing pharmaceutical agents (drugs) that can be introduced into the vagina in a simple manner without medical assistance. For example, NUVARING® (etonogestrel/ethinyl estradiol vaginal ring) was designed to be used during single 28-day cycles. NUVARING® is discarded at 21 days and a new ring inserted at the beginning of the next 28-day cycle. While the product provides a month of contraception without having to remember a daily pill, there is still a need for regular prescription refills during the year.

### SUMMARY

In a first aspect, the present disclosure provides a method of preventing pregnancy in a female of reproductive potential over a consecutive 364-day product-use period, the method comprising:
(a) inserting into the vagina of the female a vaginal ring system, the ring system comprising:
   i. a silicone elastomer ring body having a platinum concentration of approximately 3 ppm to approximately 10 ppm and a hydride/vinyl ratio from approximately 1:1 to approximately 1.3:1 before curing; and
      a. a first cylindrical channel adapted to receive a first cylindrical core; and
      b. a second cylindrical channel adapted to receive a second cylindrical core;
   ii. a first cylindrical core disposed within the first cylindrical channel, wherein the first cylindrical core comprises first and second condensation-cure silicone elastomers, dibutyltin dilaurate, and a viscosity agent selected from the group consisting of diatomaceous earth, cellulose, talc, and silica, and wherein the first cylindrical core comprises approximately 50% segesterone acetate by mass;
   iii. a second cylindrical core disposed within the second cylindrical channel, the second cylindrical core comprising a third condensation-cure silicone elastomer, wherein the second and third condensation-cure silicone elastomers are optionally the same, and dibutyltin dilaurate, and wherein the second cylindrical core comprises approximately 40% segesterone acetate by mass and approximately 12% ethinyl estradiol by mass; and
   iv. approximately 103 mg of segesterone acetate and approximately 17.4 mg of ethinyl estradiol, wherein both the segesterone acetate and ethinyl estradiol are contained within the cores of the vaginal ring system;
      wherein:
      the first and second cylindrical cores have a volume ratio of about 11:18; and
(b) retaining the ring system in the vagina for 364 consecutive days, wherein the method achieves in a serum sample of a female subject one or more of the following:
   i) a segesterone acetate concentration in serum on day 364 of approximately 100 pmol/L to approximately 350 pmol/L; and
   ii) an ethinyl estradiol concentration in serum on day 364 of approximately 10 pmol/L to approximately 100 pmol/L.

In a first embodiment of the first aspect, the method achieves in a serum sample of a female subject one or both of:
i) a segesterone acetate concentration in serum on day 364 of approximately 184 pmol/L on average; and
ii) an ethinyl estradiol concentration in serum on day 364 of approximately 43 pmol/L on average.

In a second embodiment of the first aspect, the method further achieves in a serum sample of a female subject one or both of the following during the 364-day product-use period:
iii) a segesterone acetate Cₘₐₓ of approximately 3,097±850 pmol/L of segesterone acetate over the 364-day product-use period; and
iv) an ethinyl estradiol Cₘₐₓ of approximately 435±131 pmol/L of ethinyl estradiol over the 364-day product-use period.

In a second aspect, the present disclosure provides a method of preventing pregnancy in a female of reproductive potential, the method comprising:
(a) initially inserting into the vagina of the female a reusable vaginal ring system; and
(b) removing the vaginal ring system on the day following the end of a consecutive 80- to 90-day product-use period;
   wherein the vaginal ring system comprises:
i. a silicone elastomer ring body having a platinum concentration of approximately 3 ppm to approximately 10 ppm and a hydride/vinyl ratio from approximately 1:1 to approximately 1.3:1 before curing; and
   a. a first cylindrical channel adapted to receive a first cylindrical core; and
   b. a second cylindrical channel adapted to receive a second cylindrical core;
ii. a first cylindrical core disposed within the first cylindrical channel, wherein the first cylindrical core comprises first and second condensation-cure silicone elastomers, dibutyltin dilaurate, and a viscosity agent selected from the group consisting of diatomaceous earth, cellulose, talc, and silica, and wherein the first cylindrical core comprises approximately 50% segesterone acetate by mass;
iii. a second cylindrical core disposed within the second cylindrical channel, the second cylindrical core comprising a third condensation-cure silicone elastomer, wherein the second and third condensation-cure silicone elastomers are optionally the same, and dibutyltin dilaurate, and wherein the second cylindrical core comprises approximately 40% segesterone acetate by mass and approximately 12% ethinyl estradiol by mass; and
iv. approximately 103 mg of segesterone acetate and approximately 17.4 mg of ethinyl estradiol, wherein both the segesterone acetate and ethinyl estradiol are contained within the cores of the vaginal ring system;
   wherein:
the first and second cylindrical cores have a volume ratio of approximately 11:18;
wherein the method achieves in a serum sample of a female subject one or both of the following:
i) an average segesterone acetate concentration in serum during the product-use period of approximately 350 pmol/L to approximately 425 pmol/L; and
ii) an average ethinyl estradiol concentration in serum during the product-use period of approximately 75 pmol/L to approximately 125 pmol/L.

In a first embodiment of the second aspect, the method further comprises:
(c) storing the removed vaginal ring system for a removal period of approximately 3 to 7 days including the removal date of step (b), wherein the product-use period and removal period together comprise a product-use cycle; and
(d) repeating steps (a), (b), and (c), for a total of up to four product-use cycles.

In a second embodiment of the second aspect, the method achieves in a serum sample of a female subject one or more of the following:
i) an average segesterone acetate concentration in serum during the second quarterly product-use period of approximately 275 pmol/L to approximately 350 pmol/L;
ii) an average segesterone acetate concentration in serum during the third quarterly product-use period of approximately 225 to approximately 300 pmol/L of a third quarterly product-use period;
iii) an average segesterone acetate concentration in serum during the fourth quarterly product-use period of approximately 175 to approximately 250 pmol/L;
iv) an average ethinyl estradiol concentration in serum during the second quarterly product-use period of approximately 75 pmol/L to approximately 100 pmol/L;
v) an average ethinyl estradiol concentration in serum during the third quarterly product-use period of approximately 55 to approximately 75 pmol/L; and
vi) an average ethinyl estradiol concentration in serum during the fourth quarterly product-use period approximately 40 to approximately 60 pmol/L.

In a third embodiment of the second aspect, the method further achieves in a serum sample of a female subject one or both of the following:
iii) an average segesterone acetate AUC_{1F-1L} of approximately 725,000 pmol*h/L to approximately 825,000 pmol*h/L of a first product-use period; and
iv) an average ethinyl estradiol AUC_{1F-1L} of approximately 190,000 pmol*h/L to approximately 240,000 pmol*h/L of a first product-use period.

In a fourth embodiment of the second aspect, the method further achieves in a serum sample of a female subject one or more of the following:
i) an average segesterone acetate AUC_{2F-2L} of approximately 600,000 pmol*h/L to approximately 675,000 pmol*h/L;
ii) an average segesterone acetate AUC_{3F-3L} of approximately 480,000 pmol*h/L to approximately 575,000 pmol*h/L;
iii) a segesterone acetate AUC_{4F-4L} of approximately 400,000 pmol*h/L to approximately 510,000 pmol*h/L;
iv) an average ethinyl estradiol AUC_{2F-2L} of approximately 150,000 pmol*h/L to approximately 200,000 pmol*h/L;
v) an average ethinyl estradiol AUC_{3F-3L} of approximately 100,000 pmol*h/L to approximately 150,000 pmol*h/L; and
vi) an average ethinyl estradiol AUC_{4F-4L} of approximately 75,000 pmol*h/L to approximately 125,000 pmol*h/L.

In a fifth embodiment of the second aspect, the method further achieves in a serum sample of a female subject one or both of the following:
i) a segesterone acetate Cₘₐₓ of approximately 3,097±850 pmol/L of segesterone acetate over the first product-use period; and
ii) an ethinyl estradiol Cₘₐₓ of approximately 435±131 pmol/L of ethinyl estradiol over the first product-use period.

In a sixth embodiment of the second aspect, the product-use period consists of 80 consecutive days.

In a seventh embodiment of the second aspect, the product-use period consists of 81 consecutive days.

In an eighth embodiment of the second aspect, the product-use period consists of 82 consecutive days.

In a ninth embodiment of the second aspect, the product-use period consists of 83 consecutive days.

In a tenth embodiment of the second aspect, the product-use period consists of 84 consecutive days.

In an eleventh embodiment of the second aspect, the product-use period consists of 85 consecutive days.

In a twelfth embodiment of the second aspect, the product-use period consists of 86 consecutive days.

In a thirteenth embodiment of the second aspect, the product-use period consists of 87 consecutive days.

In a fourteenth embodiment of the second aspect, the product-use period consists of 88 consecutive days.

In a fifteenth embodiment of the second aspect, the product-use period consists of 89 consecutive days.

In a sixteenth embodiment of the second aspect, the product-use period consists of 90 consecutive days.

In a seventeenth embodiment of the second aspect, the removal period consists of 3 consecutive days.

In an eighteenth embodiment of the second aspect, the removal period consists of 4 consecutive days.

In a nineteenth embodiment of the second aspect, the removal period consists of 5 consecutive days.

In a twentieth embodiment of the second aspect, the removal period consists of 6 consecutive days.

In a twenty-first embodiment of the second aspect, the removal period consists of 7 consecutive days.

In some embodiments of the aspects described herein, approximately 80% to approximately 90% of the ethinyl estradiol is recoverable from the ring system after approximately 18 months of storage at 25° C. and 60% relative humidity and wherein no more than approximately 10% to approximately 20% of the ethinyl estradiol undergoes hydrosilylation with unreacted hydrosilane in the ring body after approximately 18 months of storage at 25° C. and 60% relative humidity.

In some embodiments of the aspects described herein, the first cylindrical core has a length of approximately 11 mm.

In some embodiments of the aspects described herein, the second cylindrical core has a length of approximately 18 mm.

In some embodiments of the aspects described herein, the first cylindrical core has a diameter of approximately 3 mm.

In some embodiments of the aspects described herein, the second cylindrical core has a diameter of approximately 3 mm.

In some embodiments of the aspects described herein, the first and second cylindrical channels each have a diameter of approximately 3 mm.

In some embodiments of the aspects described herein, the first and second cylindrical cores are secured in the first and second channels, respectively, with an adhesive.

In some embodiments of the aspects described herein, the first cylindrical core is substantially longitudinally centered in the first cylindrical channel, further wherein the second cylindrical core is substantially longitudinally centered within the second cylindrical channel.

In embodiments of the aspects described herein,
a. the first cylindrical core has a first end face and a second end face, wherein the first cylindrical core is fully disposed within the first cylindrical channel;
b. the second cylindrical core has a first end face and a second end face, wherein the second cylindrical core is fully disposed within the second cylindrical channel; and
c. an end face of the first cylindrical core is substantially coplanar with an end face of the second cylindrical core.

In some embodiments of the aspects described herein, the first cylindrical channel and the second cylindrical channels each have lengths of approximately 27 mm.

In some embodiments of the aspects described herein, the first and second cylindrical channels are substantially parallel to each other.

In some embodiments of the aspects described herein, any void spaces in the first and second cylindrical channels not occupied by the first and second cylindrical cores are filled with adhesive.

In some embodiments of the aspects described herein, the ring body has an outer diameter, an inner diameter, and a cross-sectional diameter.

In some embodiments of the aspects described herein, the outer diameter is approximately 56 mm.

In some embodiments of the aspects described herein, the inner diameter is approximately 40 mm.

In some embodiments of the aspects described herein, the cross-sectional diameter is approximately 8.4 mm.

In some embodiments of the aspects described herein, the silicone elastomer ring body has a platinum concentration of approximately 4 ppm to approximately 9 ppm before curing.

In some embodiments of the aspects described herein, the silicone elastomer ring body has a platinum concentration of approximately 5 ppm to approximately 8 ppm before curing.

In some embodiments of the aspects described herein, the silicone elastomer ring body has a shore A hardness of approximately 25 to approximately 30, a mean fatigue parallel to the cores of approximately 95% and a mean fatigue perpendicular to the cores of approximately 98%.

In some embodiments of the aspects described herein, the first cylindrical core is impregnated with a first amount of segesterone acetate particles having a particle size distribution: D90 of not more than 10 microns and a D50 of not more than 5 microns; further wherein in the second cylindrical core is impregnated with a second amount of segesterone acetate particles and an amount of ethinyl estradiol particles, wherein the ethinyl estradiol particles have a particle size distribution of 100% max 15 microns, 99% max 12.5 microns, 95% max 10 microns and max 40% less than or equal to 1.3 microns.

In some embodiments of the aspects described herein, at least 75% of the segesterone acetate comprises segesterone acetate Polymorphic form I.

In some embodiments of the aspects described herein, the segesterone acetate comprises up to 25% segesterone acetate Polymorphic form II.

In some embodiments of the aspects described herein, after 18 months of storage of the vaginal system, at least one degradation product selected from the group consisting of 6α-OH-EE, 6β-OH-EE, 6α-OH-NES, 6β-OH-NES, 17β-estradiol, NES ST-alcohol, NES iso-ST-alcohol, 6,7-didehydro-EE & 9,11-didehydro-EE, estrone, Δ⁶-NES, Iso-NES, 3-enolacetate-NES, 3-methoxy-NES, and combinations thereof, is detectable but does not account for more than 5% of ring extractables as measured by HPLC.

In some embodiments of the aspects described herein, the at least one degradation product is detectable but does not account for more than 1% of ring extractables as measured by HPLC.

In some embodiments of the aspects described herein, the ring body comprises approximately 4% TiO₂ by weight.

In another aspect, the present disclosure provides a method of birth control that uses a vaginal system compatible with male condoms made from natural rubber latex, polyisoprene, or polyurethane and is suitable for four quarterly or one annual product-use cycle.

In certain embodiments of this aspect the first product-use cycle begins with an initial insertion of the vaginal system on any day of the female's menstrual cycle. In some embodiments, the first product-use period begins with an initial insertion of the vaginal system on day 2, 3, 4, or 5 of the female's menstrual cycle.

In certain embodiments, each product-use cycle comprises a removal period during which the vaginal system is outside of the female's vagina. In some embodiments, the removal period begins with removal of the vaginal system on the day after the end of the product-use period. In some embodiments, the vaginal system is stored at room temperature during the removal period.

### BRIEF DESCRIPTION OF THE DRAWINGS/FIGURES

FIG. 1A and FIG. 1B are diagrams of the vaginal system disclosed herein.

FIG. 2 is an XRPD comparison of the ethinyl estradiol/segesterone acetate core to the historical patterns of segesterone acetate Polymorphic forms I and II.

FIG. 3 is an XRPD comparison of ethinyl estradiol to the calculated patterns of ethinyl estradiol hemihydrate and anhydrous ethinyl estradiol.

FIG. 4 is a schematic of the platinum-catalyzed reaction that forms the ring body elastomer.

FIG. 5 is a schematic of the reaction between ethinyl estradiol and components of ring body elastomer.

FIG. 6A is a ¹³C-solid state NMR spectra of 17α-ethinyl-¹³C₂-estradiol (20,21-¹³C₂ labelled; 99.1% isotopic enrichment)

FIG. 6B is a ¹³C-solid state NMR spectra of NuSil™ MED4-4224 (9:1 mixture of Part A:Part B)

FIG. 7A is a ¹³C-solid state NMR spectra of an EE-¹³C₂ silicone sample before solvent extraction.

FIG. 7B is a ¹³C-solid state NMR spectra of an EE-¹³C₂ silicone sample after solvent extraction.

FIG. 8A is a diagram of the upper and lower rig used to measure tensile strength and elongation.

FIG. 8B is a diagram showing tensile measurement orientations parallel and perpendicular to the ring core.

FIG. 8C shows a ring loaded for tensile strength and elongation measurement parallel to the ring cores.

FIG. 8D shows a ring loaded for tensile strength and elongation measurement perpendicular to the ring cores.

FIG. 9A is a diagram showing compression measurement orientations parallel and perpendicular to the ring core.

FIG. 9B is a diagram showing the compression probe appliance of the compression rig.

FIG. 9C is a diagram showing the lower compression rig.

FIG. 9D is a diagram showing the lower compression rig including the nylon strap.

FIG. 9E shows a ring loaded for compression measurement parallel to the ring cores.

FIG. 9F shows a ring loaded for compression measurement perpendicular to the ring cores.

FIG. 10A shows the structures of the identified NES and EE degradation products.

FIG. 10B shows the structures of the identified NES and EE degradation products.

FIG. 10C shows the structures of the identified NES and EE degradation products.

FIG. 10D shows the structures of the identified NES and EE degradation products.

FIG. 11 shows a graphical representation of exemplary schedules for using the vaginal system described herein over 4 quarterly product-use cycles.

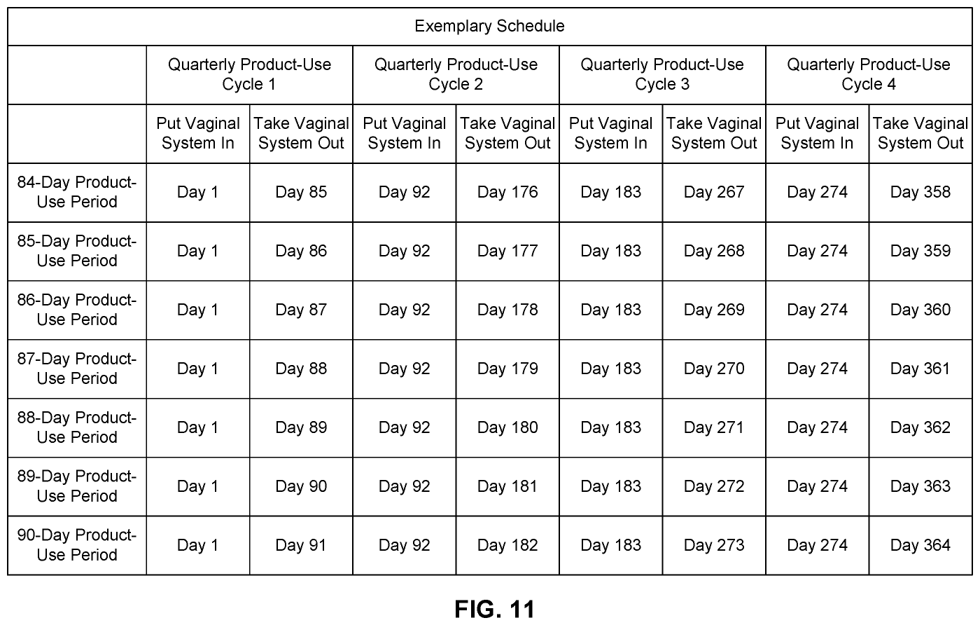

FIG. 12 shows a log-linear model of observed and predicted hormone levels over time in a pharmacokinetic study for segesterone acetate (SA).

FIG. 13 shows a log-linear model of observed and predicted hormone levels over time in a pharmacokinetic study for ethinyl estradiol (EE).

### DETAILED DESCRIPTION

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the term “approximately” refers to #10% of a noted value, except when used in conjunction with a time period measured in days, in which case “approximately” refers to +2 days.

As used herein, the term “AUC_{1F-1L}” refers to the area under the curve between the first and the last day of a first quarterly product-use period.

As used herein, the term “AUC_{2F-2L}” refers to the area under the curve between the first and the last day of a second quarterly product-use period.

As used herein, the term “AUC_{3F-3L}” refers to the area under the curve between the first and the last day of a third quarterly product-use period.

As used herein, the term “AUC_{4F-4L}” refers to the area under the curve between the first and the last day of a fourth quarterly product-use period.

The term “bioequivalent,” has the meaning defined in 21 C.F.R. § 320.1(e) and refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended-release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use and is considered medically insignificant for the drug. In practice, two products are considered bioequivalent if the 90% confidence interval of the AUC or Cₘₐₓ is within 80% to 125%.

The term “compatible” as used herein, refers to the ability of two or more items of different chemical makeup to come into repeated contact with each other over the course of an extended period, such as approximately 1 year, without a detrimental effect to any of the items coming into contact with each other over the period of time. Exemplary detrimental effects that do not occur when two or more items are compatible include, but are not limited to, a chemical reaction between the two or more items, an increase in brittleness in one or more of the items, tearing of one or more of the items, expansion or contraction of one or more of the items, breakage of one or more of the items, hardening of one or more of the items, softening of one or more of the items, erosion of one or more of the items, and/or reduced functionality of one or more of the items, such as a change in the rate of drug release from one of the items.

The term “day” as used herein, refers to a period of 24 hours.

The term “elongation” as used herein, is the amount of increase in length that occurs before a substance breaks under tension. The procedure used to measure elongation of the subject vaginal ring is described in Example 5 herein.

“Ethinyl estradiol” and “EE” as used herein, refer to the compound with the established name 19-nor-17α-pregna-1,3,5(10)-trien-20-yne-3,17-diol, molecular formula C₂₀H₂₄O₂, having the structure:

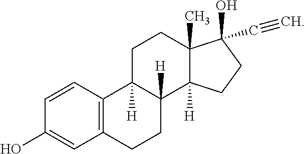

The physical form of the compound is a white to slightly yellowish-white crystalline powder. The compound is practically insoluble in water, freely soluble in alcohol, and dissolves in alkaline solution. In certain embodiments, the EE comprises a crystalline form that melts from approximately 181° C. to approximately 186° C. In some embodiments, the EE comprises a crystalline form that melts from approximately 141° C. to approximately 146° C.

The term “fatigue” as used herein, refers to the weakening of a material caused by repeatedly applied loads. The procedure used to measure the fatigue of the vaginal ring described in this disclosure is described in Example 6 herein.

The term “hydrosilation” as used herein, refers to the catalyzed addition of Si—H bonds across unsaturated bonds.

The phrase “initial insertion” as used herein, refers to the first insertion of the vaginal system into the vagina during a product-use cycle. In some embodiments of the present disclosure the initial insertion can occur on day 2, 3, 4, or 5 of the female's menstrual cycle. In some embodiments of the present disclosure, the initial insertion period can occur within 1, 2, 3, 4, or 5 days of unprotected intercourse.

The terms “menstrual cycle” and “menstrual period” as used herein, refer to the process of ovulation and menstruation in women. In one embodiment of the present disclosure, day 1 is the first day of menstruation.

The phrase “natural rubber latex” as used herein, refers to the substance derived from the milky fluid obtained from plants such as the rubber tree.

The term “polyisoprene,” as used herein, refers to a polymer of isoprene, the polymer having the structure:

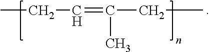

The term “polyurethane” as used herein, refers to a polymer composed of organic units joined by carbamate (urethane) links.

The phrase “product-use period” as used herein, refers to the period of consecutive days during which the vaginal system described herein is inside of a female's vagina, notwithstanding short periods of time during the use period which the vaginal system is removed from the vagina intentionally or unintentionally, such as intentionally for repositioning, to increase comfort during sex, or for any other reason, or unintentionally, such as while removing a tampon, during sex, or with straining during a bowel movement. For clarity, a product-use period does NOT include the removal period between product-use periods. In some embodiments of the present disclosure, the product-use period for the vaginal system described herein can be approximately 80, approximately 81, approximately 82, approximately 83, approximately 84, approximately 85, approximately 86, approximately 87, approximately 88, approximately 89, or approximately 90 days. In some embodiments of the present disclosure, the product-use period of the vaginal system described herein can be approximately 364 days.

The phrase “product-use cycle” as used herein, refers to the combined number of days of one product-use period and one removal period. For example, in some embodiments, the product-use cycle can be approximately 91 days and can comprise a product-use period of approximately X days and a removal period of approximately Y days, wherein X is ≥ approximately 80 and wherein X+Y≤approximately 91.

The term “quarterly” as used herein refers to a period of between approximately 80 days and approximately 90 days. In some embodiments, the term “quarterly” refers to a period of approximately 80, approximately 81, approximately 82, approximately 83, approximately 84, approximately 85, approximately 86, approximately 87, approximately 88, approximately 89, or approximately 90 days.

The terms “reinserting” and “reinsertion” as used herein, refer to any second or subsequent insertion of the vaginal system into the vagina within a given product-use cycle, and in particular, within the product-use period.

The term “relative humidity” as used herein, refers to the amount of water vapor present in the air, expressed as a percentage of the amount needed for saturation at the same temperature.

The term “reproductive potential” as used herein refers to the capacity for a female to produce offspring.

The phrase “room temperature” as used herein, refers to a temperature from 15° C. and 30° C.

The phrase “removal period” as used herein, refers to the consecutive days that the vaginal system is outside of a subject's vagina during a product-use cycle. The removal period is a non-overlapping period immediately following a product-use period and is a “dose-free” interval. That is, the subject does not receive either SA or EE during this period. In some embodiments, a withdrawal bleed can occur during the removal period. In some embodiments, the length of the removal period can be selected to last until a withdrawal bleed occurs. In accordance with such embodiments, the subject can commence a subsequent product-use cycle the day after a withdrawal bleed occurs. For example, and in certain embodiments, the subject can insert the vaginal system the day after a withdrawal bleed occurs, to thereby commence a subsequent product-use cycle. In some embodiments, the removal period can be from approximately 3 to approximately 7 days. In some embodiments, the removal period can be from approximately 4 to approximately 7 days. In some embodiments, the removal period can be from approximately 4 to approximately 6 days. In some embodiments, the removal period can be approximately 1 approximately 2, approximately 3, approximately 4, approximately 5, approximately 6, or approximately 7 days.

The phrase “secondary contraception product” as used herein refers to a product other than the vaginal system described herein that provides birth control. In one embodiment of the present disclosure the secondary contraception product does not comprise estrogen.

“Segesterone acetate,” “SA,” and “NES” as used herein refer to the compound with the established name 16-methylene-17α-acetoxy-19-nor-pregn-4-ene-3,20-dione, molecular formula C₂₃H₃₀O₄, having the structure:

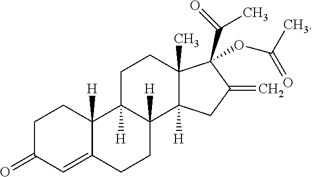

The physical form of the compound is a white, or yellowish white powder. The compound is slightly soluble in n-hexane, soluble in ethyl acetate and methanol, and freely soluble in acetone (USP classification). Segesterone acetate is sold under the trade name NESTORONE®.

The term “subject” as used herein, refers to a human female of reproductive potential.

The term “substantially pure” as used herein, refers to a polymorph of a compound which is greater than approximately 90% pure. This means that the polymorph does not contain more than approximately 10% of any other compound or any other form of the compound.

The term “tensile strength” as used herein, refers to the resistance of a substance to lengthwise stress, measured in force per unit of cross-sectional area, by the greatest load pulling in the direction of length that a given substance can bear without tearing apart. The procedure used to measure tensile strength is described in Example 5 herein.

The term “unacceptable EE burst” as used herein, refers to an EE burst of greater than or equal to approximately 0.13 mg (i.e., greater than or equal to approximately ten times the average amount of EE released per day by the vaginal system).

The term “vaginal system” as used herein, refers to a device that is inserted into the vagina and prevents pregnancy. In one embodiment of the present disclosure the vaginal system comprises a vaginal ring. In another embodiment of the present disclosure the vaginal system comprises a progestin/estrogen combined hormonal contraceptive (CHC). In another embodiment of the present disclosure the vaginal system is a segesterone acetate and ethinyl estradiol system. In another embodiment, the vaginal system is sold under the trade name ANNOVERA®.

In certain embodiments, the vaginal system of the present disclosure is a toroidal-shaped (i.e., ring-shaped), non-biodegradable, flexible, opaque white, silicone elastomer that is 56 mm in overall diameter and 8.4 mm in cross-sectional diameter. In some embodiments, the elastomer can be a methyl siloxane-based polymer. In some embodiments, the vaginal system further comprises inactive ingredients including titanium dioxide, dibutyltin dilaurate, and silicone medical adhesive.

In typical embodiments, each vaginal system is individually packaged in an aluminum pouch. Typically, the pouch consists of a laminate material comprising, from outside to inside, polyester, aluminum foil, and polyethylene. A compact case that is inert to the vaginal system can be provided for subjects to store the system.

In some embodiments, the vaginal system described herein contains from approximately 90 mg to approximately 120 mg of segesterone acetate (SA). In some embodiments, the vaginal system described herein contains from approximately 95 mg to approximately 115 mg of SA. In some embodiments, the vaginal system described herein contains from approximately 100 mg to approximately 110 mg of SA. In some embodiments, the vaginal system described herein contains approximately 103 mg of SA. In some embodiments, the vaginal system described herein contains 103 mg of SA.

In some embodiments, the vaginal system described herein contains from approximately 10 mg to approximately 25 mg of ethinyl estradiol (EE). In some embodiments, the vaginal system described herein contains from approximately 15 mg to approximately 20 mg of EE. In some embodiments, the vaginal system described herein contains approximately 17.4 mg of EE. In some embodiments, the vaginal system described herein contains 17.4 mg of EE.

In some embodiments, the vaginal system described herein contains 103 mg of SA and 17.4 mg of EE.

### Methods of Use

As noted above, it has been surprisingly discovered that the vaginal system described herein can be suitable for and used over one, two, three, or four quarterly product-use cycles or one consecutive 364-day product-use period. In certain embodiments, to commence the first product-use period, the vaginal system described herein can be inserted into the vagina of a female of reproductive potential on day 2, 3, 4, or 5 of the female's menstrual cycle. In some embodiments, the vaginal system can be retained in the vagina for the product-use period (e.g., quarterly or 364 days) to deliver contraceptively effective amounts of segesterone acetate and ethinyl estradiol over the product-use period. Further details of these embodiments are disclosed below.

### 364-Day Product-Use Period

As noted above, in accordance with some embodiments, disclosed herein are methods of preventing pregnancy in, or providing birth control to, a female of reproductive potential over a consecutive 364-day period, the methods comprising:
(a) inserting into the vagina of the female a vaginal ring system described herein; and
(b) retaining the ring system in the vagina for a consecutive 364-day product-use period.

In some embodiments, on the last day of a 364-day product-use period, the vaginal system can be removed from the vagina of the female and discarded.

As noted above and discussed herein, a consecutive 364-day product-use period can include short periods of time during which the vaginal system is removed from the vagina intentionally or unintentionally, such as intentionally for repositioning, to increase comfort during sex, or for any other reason, or unintentionally, such as while removing a tampon, during sex, or with straining during a bowel movement.

In certain embodiments, the method can achieve in a serum sample of a female subject a segesterone acetate concentration in serum on day 364 of approximately 100 pmol/L to approximately 350 pmol/L of segesterone acetate. In some embodiments, the method can achieve in a serum sample of a female subject a segesterone acetate concentration in serum on day 364 of approximately 100 pmol/L to approximately 345 pmol/L, approximately 100 pmol/L to approximately 340 pmol/L, approximately 101 pmol/L to approximately 335 pmol/L, approximately 102 pmol/L to approximately 332 pmol/L, approximately 100 pmol/L to approximately 330 pmol/L, approximately 100 pmol/L to approximately 325 pmol/L, approximately 100 pmol/L to approximately 320 pmol/L, approximately 100 pmol/L to approximately 315 pmol/L, approximately 100 pmol/L to approximately 310 pmol/L, approximately 100 pmol/L to approximately 300 pmol/L, approximately 100 pmol/L to approximately 275 pmol/L, approximately 100 pmol/L to approximately 250 pmol/L, approximately 100 pmol/L to approximately 225 pmol/L, or approximately 100 pmol/L to approximately 200 pmol/L of segesterone acetate. In some embodiments, the method can achieve in a serum sample of a female subject a segesterone acetate concentration in serum on day 364 of approximately 100 pmol/L, approximately 101 pmol/L, approximately 102 pmol/L, approximately 103 pmol/L, approximately 104 pmol/L, approximately 105 pmol/L, approximately 106 pmol/L, approximately 107 pmol/L, approximately 108 pmol/L, approximately 109 pmol/L, approximately 110 pmol/L, approximately 115 pmol/L, approximately 120 pmol/L, approximately 12 pmol/L 5, approximately 130 pmol/L, approximately 135 pmol/L, approximately 140 pmol/L, approximately 145 pmol/L, approximately 150, pmol/L approximately 155 pmol/L, approximately 160 pmol/L, approximately 161 pmol/L, approximately 162 pmol/L, approximately 163 pmol/L, approximately 164 pmol/L, approximately 165 pmol/L, approximately 166 pmol/L, approximately 167 pmol/L, approximately 168 pmol/L, approximately 169 pmol/L, approximately 170 pmol/L, approximately 171 pmol/L, approximately 172 pmol/L, approximately 173 pmol/L, approximately 174 pmol/L, approximately 175 pmol/L, approximately 176 pmol/L, approximately 177 pmol/L, approximately 178 pmol/L, approximately 179 pmol/L, approximately 180 pmol/L, approximately 181 pmol/L, approximately 182 pmol/L, approximately 183 pmol/L, approximately 184 pmol/L, approximately 185 pmol/L, approximately 186 pmol/L, approximately 187 pmol/L, approximately 188 pmol/L, approximately 189 pmol/L, approximately 190 pmol/L, approximately 191 pmol/L, approximately 192 pmol/L, approximately 193 pmol/L, approximately 194 pmol/L, approximately 195, approximately 196 pmol/L, approximately 197 pmol/L, approximately 198 pmol/L, approximately 199 pmol/L, approximately 200 pmol/L, approximately 210 pmol/L, approximately 220 pmol/L, approximately 230 pmol/L, approximately 240 pmol/L, approximately 250 pmol/L, approximately 260, approximately 270 pmol/L, approximately 280 pmol/L, approximately 290 pmol/L, approximately 300 pmol/L, approximately 310 pmol/L, approximately 320 pmol/L, approximately 330 pmol/L, approximately 340 pmol/L, or approximately 350 pmol/L of segesterone acetate.

Surprisingly, the linear regression model used to generate this data predicted that the serum SA level on day 364 would be above 100 pmol/L, the level that has previously been shown to suppress ovulation, without the 7-day “product-out” periods that are indicated in the currently approved 13 product-cycle regimen. The vaginal ring described herein can therefore provide an effective long-term, continuous, easy-to-use contraceptive option for women.

Additionally, the method can achieve in a serum sample of a female subject an ethinyl estradiol concentration in serum on day 364 of approximately 10 pmol/L to approximately 100 pmol/L of ethinyl estradiol. In some embodiments, the method can achieve in a serum sample of a female subject an ethinyl estradiol concentration in serum on day 364 of approximately 15 pmol/L to approximately 99 pmol/L, approximately 16 pmol/L to approximately 98 pmol/L, approximately 17 pmol/L to approximately 98 pmol/L, approximately 18 pmol/L to approximately 97 pmol/L, approximately 18 pmol/L to approximately 96 pmol/L, approximately 19 pmol/L to approximately 95 pmol/L, approximately 20 pmol/L to approximately 93 pmol/L, approximately 21 pmol/L to approximately 92 pmol/L, approximately 22 pmol/L to approximately 91 pmol/L, approximately 23 pmol/L to approximately 90 pmol/L, approximately 24 pmol/L to approximately 90 pmol/L, approximately 25 pmol/L to approximately 90 pmol/L, approximately 26 pmol/L to approximately 85 pmol/L, approximately 27 pmol/L to approximately 80 pmol/L, approximately 28 pmol/L to approximately 75 pmol/L, approximately 29 pmol/L to approximately 70 pmol/L, approximately 30 pmol/L to approximately 70 pmol/L, approximately 35 pmol/L to approximately 65 pmol/L, approximately 40 pmol/L to approximately 60 pmol/L, or approximately 40 pmol/L to approximately 50 pmol/L of ethinyl estradiol. In some embodiments, the method can achieve in a serum sample of a female subject an ethinyl estradiol concentration in serum on day 364 of approximately 10 pmol/L, approximately 11 pmol/L, approximately 12 pmol/L, approximately 13 pmol/L, approximately 14 pmol/L, approximately 15 pmol/L, approximately 16 pmol/L, approximately 17 pmol/L, approximately 18 pmol/L, approximately 19 pmol/L, approximately 20 pmol/L, approximately 21 pmol/L, approximately 22 pmol/L, approximately 23 pmol/L 5, approximately 24 pmol/L, approximately 25 pmol/L, approximately 26 pmol/L, approximately 27 pmol/L, approximately 28, pmol/L approximately 29 pmol/L, approximately 30 pmol/L, approximately 35 pmol/L, approximately 40 pmol/L, approximately 41 pmol/L, approximately 42 pmol/L, approximately 43 pmol/L, approximately 44 pmol/L, approximately 45 pmol/L, approximately 46 pmol/L, approximately 47 pmol/L, approximately 48 pmol/L, approximately 49 pmol/L, approximately 50 pmol/L, approximately 55 pmol/L, approximately 60 pmol/L, approximately 65 pmol/L, approximately 70 pmol/L, approximately 75 pmol/L, approximately 80 pmol/L, approximately 85 pmol/L, approximately 90 pmol/L, approximately 91 pmol/L, approximately 92 pmol/L, approximately 93 pmol/L, approximately 94 pmol/L, approximately 95 pmol/L, approximately 96 pmol/L, approximately 97 pmol/L, approximately 98 pmol/L, approximately 99 pmol/L, or approximately 100 pmol/L of ethinyl estradiol.

In some embodiments, the method can achieve in a serum sample of a female subject a segesterone acetate concentration in serum on day 364 of approximately 100 pmol/L to approximately 350 pmol/L of segesterone acetate and an ethinyl estradiol concentration in serum on day 364 of approximately 10 pmol/L to approximately 100 pmol/L of ethinyl estradiol. In some embodiments, the method can achieve in a serum sample of a female subject a segesterone acetate concentration in serum on day 364 of approximately 184 pmol/L of segesterone acetate and an ethinyl estradiol concentration in serum on day 364 of approximately 43 pmol/L of ethinyl estradiol.

Surprisingly, the linear regression model used to generate this data predicted no accumulation of serum EE levels at day 364, which is significant in because of concerns regarding venous thromboembolism (VTE) risk with continuous vaginal ring use. As higher EE levels have been observed with the continuous use of transdermal patches, the vaginal ring described herein provides a safe long-term contraceptive option for women.

Additionally or alternatively, the methods can achieve, as measured in a sample obtained from the female, a segesterone acetate Cₘₐₓ of approximately 2,241 pmol/L to approximately 3,982 pmol/L over the 364-day product-use period. For example, the method can achieve, as measured in a sample obtained from the female, a segesterone acetate Cₘₐₓ of approximately 3,097±850 pmol/L over the 364-day product-use period.

### Quarterly Product-Use Period

As noted above, in certain embodiments, the vaginal systems described herein can be suitable for and used over four quarterly product-use cycles. In accordance with these embodiments, each product-use period within the quarter can be approximately 80 to approximately 90 days. In certain embodiments, on the last day of a product-use period, the vaginal ring can be removed from the vagina and maintained outside the vagina for a removal period of approximately 1 to approximately 11 consecutive days, to complete one product-use cycle. In some embodiments, during the first, second, or third quarterly product-use cycles, the vaginal system can be cleaned and stored at room temperature during the removal period. In some embodiments, removal of the vaginal system during this removal period can comprise a withdrawal bleed. In some embodiments, on the day after the last day of a removal period, the same vaginal system can be reinserted to commence a new quarterly product-use cycle. In some embodiments, the vaginal system described herein can be re-used three times after the initial quarterly product-use cycle, for a total of up to four quarterly product-use cycles including the first product-use cycle. In some embodiments, upon completion of the fourth quarterly product-use period, the vaginal system can be removed from the vagina of the female and discarded.

Thus, in accordance with some embodiments, disclosed herein are methods of preventing pregnancy in or providing birth control to a female of reproductive potential, the methods comprising:
(a) initially inserting into the vagina of the female a reusable vaginal ring system as described herein; and
(b) removing the vaginal ring system on the day following the end of a quarterly product-use period of approximately 80, approximately 81, approximately 82, approximately 83, approximately 84, approximately 85, approximately 86, approximately 87, approximately 88, approximately 89, or approximately 90 consecutive days.

In some embodiments, the quarterly product-use period can be approximately 84 consecutive days. In some embodiments, the quarterly product-use period can be approximately 86 consecutive days. In some embodiments, the quarterly product-use period can be approximately 90 consecutive days.

In some embodiments, a product-use period of the methods described herein can include short periods of time during which the vaginal system is removed from the vagina intentionally or unintentionally, such as intentionally for repositioning, to increase comfort during sex, or for any other reason, or unintentionally, such as while removing a tampon, during sex, or with straining during a bowel movement.

In some embodiments, retaining the ring in the vagina for a product-use period of approximately 80 to approximately 90 consecutive days achieves in a serum sample of a female subject an average segesterone acetate concentration in serum during the first quarterly product-use period of approximately 350 pmol/L to approximately 425 pmol/L. In some embodiments, the method achieves in a serum sample of a female subject an average segesterone acetate concentration in serum during the first product-use period of approximately 360 pmol/L to approximately 410 pmol/L, approximately 365 pmol/L to approximately 400 pmol/L, approximately 370 pmol/L to approximately 390 pmol/L, approximately 375 pmol/L to approximately 385 pmol/L, approximately 377 pmol/L to approximately 386 pmol/L, or approximately 378 pmol/L to approximately 387 pmol/L. In some embodiments, the method achieves in a serum sample of a female subject an average segesterone acetate concentration in serum during the first product-use period of approximately 350 pmol/L, approximately 355 pmol/L, approximately 360 pmol/L, approximately 365 pmol/L, approximately 366 pmol/L, approximately 367 pmol/L, approximately 368 pmol/L, approximately 369 pmol/L, approximately 370 pmol/L, approximately 371 pmol/L, approximately 372 pmol/L, approximately 373 pmol/L, approximately 374 pmol/L, approximately 375 pmol/L, approximately 376 pmol/L, approximately 377 pmol/L, approximately 378 pmol/L, approximately 379 pmol/L, approximately 380 pmol/L, approximately 381 pmol/L, approximately 382 pmol/L, approximately 383 pmol/L, approximately 384 pmol/L, approximately 385 pmol/L, approximately 386 pmol/L, approximately 387 pmol/L, approximately 388 pmol/L, approximately 389 pmol/L, approximately 390 pmol/L, approximately 395 pmol/L, approximately 400 pmol/L, approximately 405 pmol/L, approximately 410 pmol/L, approximately 415 pmol/L, approximately 420 pmol/L, or approximately 425 pmol/L.

In some embodiments, retaining the ring in the vagina for a product-use period of approximately 80 to approximately 90 consecutive days achieves in a serum sample of a female subject an average ethinyl estradiol concentration in serum during the first product-use period of approximately 75 pmol/L to approximately 125 pmol/L. In some embodiments, the method achieves in a serum sample of a female subject an average ethinyl estradiol concentration in serum during the product-use period of approximately 80 pmol/L to approximately 120 pmol/L, approximately 85 pmol/L to approximately 115 pmol/L, approximately 90 pmol/L to approximately 115 pmol/L, approximately 95 pmol/L to approximately 115 pmol/L, approximately 107 pmol/L to approximately 110 pmol/L, or approximately 106 pmol/L to approximately 110 pmol/L. In some embodiments, the method can achieve in a serum sample of a female subject an average ethinyl estradiol concentration in serum during the first product-use period of approximately 80 pmol/L, approximately 85 pmol/L, approximately 90 pmol/L, approximately 95 pmol/L, approximately 96 pmol/L, approximately 97 pmol/L, approximately 98 pmol/L, approximately 99 pmol/L, approximately 100 pmol/L, approximately 101 pmol/L, approximately 102 pmol/L, approximately 103 pmol/L, approximately 104 pmol/L, approximately 105 pmol/L, approximately 106 pmol/L, approximately 107 pmol/L, approximately 108 pmol/L, approximately 109 pmol/L, approximately 110 pmol/L, approximately 111 pmol/L, approximately 112 pmol/L, approximately 113 pmol/L, approximately 114 pmol/L, approximately 115 pmol/L, approximately 116 pmol/L, approximately 117 pmol/L, approximately 118 pmol/L, approximately 119 pmol/L, approximately 120 pmol/L, approximately 121 pmol/L, approximately 122 pmol/L, approximately 123 pmol/L, approximately 124 pmol/L, or approximately 125 pmol/L.

In some embodiments, the method achieves in a serum sample of a female subject a segesterone acetate AUC_{1F-1L} of approximately 725,000 pmol*h/L to approximately 825,000 pmol*h/L. In some embodiments, the method can achieve in a serum sample of a female subject a segesterone acetate AUC_{1F-1L} of approximately 735,000 pmol*h/L to approximately 835,000 pmol*h/L, approximately 740,000 pmol*h/L to approximately 830,000 pmol*h/L, approximately 745,000 pmol*h/L to approximately 820,000 pmol*h/L, approximately 750,000 pmol*h/L to approximately 800,000 pmol*h/L, approximately 750,000 pmol*h/L to approximately 780,000 pmol*h/L or approximately 750,000 pmol*h/L to approximately 755,000 pmol*h/L.

In some embodiments, the method can achieve in a serum sample of a female subject an ethinyl estradiol AUC_{1F-1L} of approximately 190,000 pmol*h/L to approximately 240,000 pmol*h/L. In some embodiments, the method can achieve in a serum sample of a female subject an ethinyl estradiol AUC_{1F-1L} of approximately 195,000 pmol*h/L to approximately 235,000 pmol*h/L, approximately 200,000 pmol*h/L to approximately 230,000 pmol*h/L, approximately 215,000 pmol*h/L to approximately 235,000 pmol*h/L, approximately 205,000 pmol*h/L to approximately 225,000 pmol*h/L, or approximately 210,000 pmol*h/L to approximately 220,000 pmol*h/L.

Additionally, the methods can achieve, as measured in a sample obtained from the female, a segesterone acetate Cₘₐₓ of approximately 3,097±850 pmol/L over the first quarterly product-use period.

Additionally or alternatively, the method can achieve, as measured in a sample obtained from the female, an ethinyl estradiol Cₘₐₓ of approximately 435±131 pmol/L of ethinyl estradiol over the first quarterly product-use period.

In certain embodiments, the vaginal system can be maintained outside the vagina for a removal period of approximately 1 to approximately 11 days, or to complete a product-use cycle. After completion of the first, second, and third quarterly product-use cycles, the same vaginal system can be reinserted into the vagina to commence a subsequent product-use period. Thus, in some embodiments, the quarterly product-use period methods further comprises:
(c) storing the removed vaginal ring system for a removal period of approximately 1 to approximately 11 days (including the removal date of step (b)), wherein the product-use period and removal period together comprise a product-use cycle; and
(d) repeating steps (a), (b), and (c), for a total of up to four quarterly product-use cycles per vaginal system.

In some embodiments, the present disclosure provides a method of preventing pregnancy in or providing birth control to a female, of reproductive potential the method comprising (a) initially inserting into the vagina of the female a reusable vaginal ring system as described herein; (b) removing the vaginal ring system on the day following the end of a quarterly product-use period of approximately 84 consecutive days; (c) storing the removed vaginal ring system for a removal period of approximately 1, approximately 2, approximately 3, approximately 4, approximately 5, approximately 6, approximately 7, approximately 8, approximately 9, approximately 10, or approximately 11 days including the removal date of step (b), wherein the product-use period and removal period together comprise a product-use cycle; and repeating steps (a), (b), and (c), for a total of up to four quarterly product-use cycles. In other embodiments, step (b) comprises removing the vaginal ring system on the day following the end of a quarterly product-use period of approximately 86 consecutive days. In other embodiments, step (b) comprises removing the vaginal ring system on the day following the end of a quarterly product-use period of approximately 90 consecutive days.

In some embodiments, the method can achieve in a serum sample of a female subject an average segesterone acetate concentration in serum during the second quarterly product-use period of approximately 275 pmol/L to approximately 350 pmol/L. In some embodiments, the method can achieve in a serum sample of a female subject an average segesterone acetate concentration in serum during the second quarterly product-use period of approximately 280 pmol/L to approximately 345 pmol/L, approximately 290 pmol/L to approximately 340 pmol/L, approximately 295 pmol/L to approximately 335 pmol/L, approximately 300 pmol/L to approximately 330 pmol/L, approximately 308 pmol/L to approximately 316 pmol/L, approximately 310 pmol/L to approximately 325 pmol/L, or approximately 317 pmol/L to approximately 320 pmol/L. In some embodiments, the method can achieve in a serum sample of a female subject an average segesterone acetate concentration in serum during the second quarterly product-use period of approximately 275 pmol/L, approximately 280 pmol/L, approximately 285 pmol/L, approximately 290 pmol/L, approximately 295 pmol/L, approximately 300 pmol/L, approximately 305 pmol/L, approximately 310 pmol/L, approximately 311 pmol/L, approximately 312 pmol/L, approximately 313 pmol/L, approximately 314 pmol/L, approximately 315 pmol/L, approximately 316 pmol/L, approximately 317 pmol/L, approximately 318 pmol/L, approximately 319 pmol/L, approximately 320 pmol/L, approximately 321 pmol/L, approximately 322 pmol/L, approximately 323 pmol/L, approximately 324 pmol/L, approximately 325 pmol/L, approximately 330 pmol/L, approximately 335 pmol/L, approximately 340 pmol/L, approximately 345 pmol/L, or approximately 350 pmol/L.

In some embodiments, the method can achieve in a serum sample of a female subject an average ethinyl estradiol concentration in serum during the second quarterly product-use period of approximately 75 pmol/L to approximately 100 pmol/L. In some embodiments, the method can achieve in a serum sample of a female subject an average ethinyl estradiol concentration in serum during the second quarterly product-use period of approximately 80 pmol/L to approximately 95 pmol/L, approximately 81 pmol/L to approximately 90 pmol/L, approximately 82 pmol/L to approximately 89 pmol/L, approximately 83 pmol/L to approximately 88 pmol/L, approximately 84 pmol/L to approximately 86 pmol/L, approximately 85 pmol/L to approximately 87 pmol/L, or approximately 83 pmol/L to approximately 85 pmol/L. In some embodiments, the method can achieve in a serum sample of a female subject an average ethinyl estradiol concentration in serum during the second quarterly product-use period of approximately 75 pmol/L, approximately 80 pmol/L, approximately 81 pmol/L, approximately 82 pmol/L, approximately 83 pmol/L, approximately 84 pmol/L, approximately 85 pmol/L, approximately 86 pmol/L, approximately 87 pmol/L, approximately 88 pmol/L, approximately 89 pmol/L, approximately 90 pmol/L, approximately 95 pmol/L, or approximately 100 pmol/L.

In some embodiments, the method can achieve in a serum sample of a female subject a segesterone acetate AUC_{2F-2L} of approximately 600,000 pmol*h/L to approximately 675,000 pmol*h/L. In some embodiments, the method can achieve in a serum sample of a female subject a segesterone acetate AUC_{2F-2L} of approximately 640,000 pmol*h/L to approximately 681,000 pmol*h/L, approximately 615,000 pmol*h/L to approximately 670,000 pmol*h/L, approximately 618,000 pmol*h/L to approximately 665,000 pmol*h/L, approximately 619,000 pmol*h/L to approximately 660,000 pmol*h/L, or approximately 620,000 pmol*h/L to approximately 658,000 pmol*h/L.

In some embodiments, the method can achieve in a serum sample of a female subject an ethinyl estradiol AUC_{2F-2L} of approximately 150,000 pmol*h/L to approximately 200,000 pmol*h/L. In some embodiments, the method can achieve in a serum sample of a female subject an ethinyl estradiol AUC_{2F-2L} of approximately 155,000 pmol*h/L to approximately 195,000 pmol*h/L, approximately 158,000 pmol*h/L to approximately 190,000 pmol*h/L, approximately 160,000 pmol*h/L to approximately 185,000 pmol*h/L, approximately 165,000 pmol*h/L to approximately 180,000 pmol*h/L, or approximately 170,000 pmol*h/L to approximately 186,000 pmol*h/L.

In some embodiments, the method can achieve in a serum sample of a female subject an average segesterone acetate concentration in serum during the third quarterly product-use period of approximately 225 pmol/L to approximately 300 pmol/L. In some embodiments, the method can achieve in a serum sample of a female subject an average segesterone acetate concentration in serum during the third quarterly product-use period of approximately 230 pmol/L to approximately 290 pmol/L, approximately 235 pmol/L to approximately 285 pmol/L, approximately 240 pmol/L to approximately 280 pmol/L, approximately 240 pmol/L to approximately 270 pmol/L, approximately 255 pmol/L to approximately 265 pmol/L, approximately 261 pmol/L to approximately 264 pmol/L, or approximately 252 pmol/L to approximately 258 pmol/L. In some embodiments, the method can achieve in a serum sample of a female subject an average segesterone acetate concentration in serum during the third quarterly product-use period of approximately 225 pmol/L, approximately 230 pmol/L, approximately 235 pmol/L, approximately 240 pmol/L, approximately 245 pmol/L, approximately 250 pmol/L, approximately 251 pmol/L, approximately 252 pmol/L, approximately 253 pmol/L, approximately 254 pmol/L, approximately 255 pmol/L, approximately 256 pmol/L, approximately 257 pmol/L, approximately 258 pmol/L, approximately 259 pmol/L, approximately 260 pmol/L, approximately 261 pmol/L, approximately 262 pmol/L, approximately 263 pmol/L, approximately 264 pmol/L, approximately 265 pmol/L, approximately 266 pmol/L, approximately 267 pmol/L, approximately 268 pmol/L, approximately 269 pmol/L, approximately 270 pmol/L, approximately 275 pmol/L, approximately 280 pmol/L, approximately 290 pmol/L, or approximately 300 pmol/L.

In some embodiments, the method can achieve in a serum sample of a female subject an average ethinyl estradiol concentration in serum during the third quarterly product-use period of approximately 55 pmol/L to approximately 75 pmol/L. In some embodiments, the method can achieve in a serum sample of a female subject an average ethinyl estradiol concentration in serum during the third quarterly product-use period of approximately 57 pmol/L to approximately 72 pmol/L, approximately 60 pmol/L to approximately 70 pmol/L, approximately 65 pmol/L to approximately 69 pmol/L, approximately 66 pmol/L to approximately 68 pmol/L, or approximately 64 pmol/L to approximately 66 pmol/L. In some embodiments, the method can achieve in a serum sample of a female subject an average ethinyl estradiol concentration in serum during the third quarterly product-use period of approximately 55 pmol/L, approximately 60 pmol/L, approximately 61 pmol/L, approximately 62 pmol/L, approximately 63 pmol/L, approximately 64 pmol/L, approximately 65 pmol/L, approximately 66 pmol/L, approximately 67 pmol/L, approximately 68 pmol/L, approximately 69 pmol/L, approximately 70 pmol/L, approximately 71 pmol/L, approximately 72 pmol/L, approximately 73 pmol/L, approximately 74 pmol/L, or approximately 75 pmol/L.

In some embodiments, the method can achieve in a serum sample of a female subject a segesterone acetate AUC_{3F-3L} of approximately 480,000 pmol*h/L to approximately 575,000 pmol*h/L. In some embodiments, the method can achieve in a serum sample of a female subject a segesterone acetate AUC_{3F-3L} of approximately 485,000 pmol*h/L to approximately 570,000 pmol*h/L, approximately 490,000 pmol*h/L to approximately 560,000 pmol*h/L, approximately 495,000 pmol*h/L to approximately 555,000 pmol*h/L, approximately 500,000 pmol*h/L to approximately 550,000 pmol*h/L, or approximately 534,000 pmol*h/L to approximately 585,000 pmol*h/L.

In some embodiments, the method can achieve in a serum sample of a female subject an ethinyl estradiol AUC_{3F-3L} of approximately 100,000 pmol*h/L to approximately 150,000 pmol*h/L. In some embodiments, the method can achieve in a serum sample of a female subject an ethinyl estradiol AUC_{3F-3L} of approximately 105,000 pmol*h/L to approximately 148,000 pmol*h/L, approximately 110,000 pmol*h/L to approximately 147,000 pmol*h/L, approximately 115,000 pmol*h/L to approximately 146,000 pmol*h/L, approximately 120,000 pmol*h/L to approximately 146,000 pmol*h/L, approximately 125,000 pmol*h/L to approximately 150,000 pmol*h/L, or approximately 125,000 pmol*h/L to approximately 145,000 pmol*h/L.

In some embodiments, the method can achieve in a serum sample of a female subject an average segesterone acetate concentration in serum during the fourth quarterly product-use period of approximately 175 pmol/L to approximately 250 pmol/L. In some embodiments, the method can achieve in a serum sample of a female subject an average segesterone acetate concentration in serum during the fourth quarterly product-use period of approximately 180 pmol/L to approximately 245 pmol/L, approximately 185 pmol/L to approximately 240 pmol/L, approximately 190 pmol/L to approximately 235 pmol/L, approximately 195 pmol/L to approximately 230 pmol/L, approximately 200 pmol/L to approximately 225 pmol/L, approximately 210 pmol/L to approximately 225 pmol/L, approximately 214 pmol/L to approximately 219 pmol/L, or approximately 207 pmol/L to approximately 212 pmol/L. In some embodiments, the method can achieve in a serum sample of a female subject an average segesterone acetate concentration in serum during the fourth quarterly product-use period of approximately 175 pmol/L, approximately 180 pmol/L, approximately 185 pmol/L, approximately 190 pmol/L, approximately 195 pmol/L, approximately 200 pmol/L, approximately 201 pmol/L, approximately 202 pmol/L, approximately 203 pmol/L, approximately 204 pmol/L, approximately 205 pmol/L, approximately 206 pmol/L, approximately 207 pmol/L, approximately 208 pmol/L, approximately 209 pmol/L, approximately 210 pmol/L, approximately 211 pmol/L, approximately 212 pmol/L, approximately 213 pmol/L, approximately 214 pmol/L, approximately 215 pmol/L, approximately 216 pmol/L, approximately 217 pmol/L, approximately 218 pmol/L, approximately 219 pmol/L, approximately 220 pmol/L, approximately 221 pmol/L, approximately 222 pmol/L, approximately 223 pmol/L, approximately 224 pmol/L, approximately 250 pmol/L, or approximately 250 pmol/L.

In some embodiments, the method can achieve in a serum sample of a female subject an average ethinyl estradiol concentration in serum during the fourth quarterly product-use period of approximately 40 pmol/L to approximately 60 pmol/L. In some embodiments, the method can achieve in a serum sample of a female subject an average ethinyl estradiol concentration in serum during the fourth quarterly product-use period of approximately 45 pmol/L to approximately 59 pmol/L, approximately 47 pmol/L to approximately 58 pmol/L, approximately 49 pmol/L to approximately 57 pmol/L, approximately 51 pmol/L to approximately 55 pmol/L, or approximately 52 pmol/L to approximately 53 pmol/L. In some embodiments, the method can achieve in a serum sample of a female subject an average ethinyl estradiol concentration in serum during the fourth quarterly product-use period of approximately 40 pmol/L, approximately 42 pmol/L, approximately 44 pmol/L, approximately 46 pmol/L, approximately 48 pmol/L, approximately 50 pmol/L, approximately 51 pmol/L, approximately 52 pmol/L, approximately 53 pmol/L, approximately 54 pmol/L, approximately 55 pmol/L, approximately 56 pmol/L, approximately 57 pmol/L, approximately 58 pmol/L, approximately 59 pmol/L, or approximately 60 pmol/L.

In some embodiments, the method can achieve in a serum sample of a female subject a segesterone acetate AUC_{4F-4L} of approximately 400,000 pmol*h/L to approximately 510,000 pmol*h/L. In some embodiments, the method can achieve in a serum sample of a female subject a segesterone acetate AUC_{4F-4L} of approximately 425,000 pmol*h/L to approximately 505,000 pmol*h/L, approximately 440,000 pmol*h/L to approximately 505,000 pmol*h/L, approximately 445,000 pmol*h/L to approximately 502,000 pmol*h/L, approximately 450,000 pmol*h/L to approximately 500,000 pmol*h/L, or approximately 425,000 pmol*h/L to approximately 482,000 pmol*h/L.

In some embodiments, the method can achieve in a serum sample of a female subject an ethinyl estradiol AUC_{4F-4L} of approximately 75,000 pmol*h/L to approximately 125,000 pmol*h/L. In some embodiments, the method can achieve in a serum sample of a female subject an ethinyl estradiol AUC_{4F-4L} of approximately 80,000 pmol*h/L to approximately 120,000 pmol*h/L, approximately 95,000 pmol*h/L to approximately 117,000 pmol*h/L, approximately 85,000 pmol*h/L to approximately 115,000 pmol*h/L, approximately 87,000 pmol*h/L to approximately 112,000 pmol*h/L, or approximately 90,000 pmol*h/L to approximately 110,000 pmol*h/L.

As in the 364-day product-use period analysis, the linear regression model used to generate this data has also predicted no accumulation of serum EE levels after each quarterly product-use period. The model has also predicted that the serum SA level after the last quarterly product-use period is above 100 pmol/L, the level that has previously been shown to suppress ovulation, with fewer “product-out” periods than indicated in the approved 13 product-cycle regimen. This is surprising, because the ring has been shown to be effective when longer product-use periods are employed without requiring extended removal periods to compensate for the longer periods, The vaginal ring described herein can therefore provide flexible, safe, long-term contraceptive options for women.

Thus, in some embodiments, disclosed herein is a method of preventing pregnancy in or providing birth control to a female of reproductive potential, the method comprising:
(a) inserting into the vagina of the female a reusable vaginal ring system described herein;
(b) removing the vaginal ring system on the day following the end of a quarterly product-use period of approximately 84 consecutive days;
(c) storing the removed vaginal ring system for a removal period of 1 to 11 days including the removal date of step (b), wherein the product-use period and removal period together comprise a product-use cycle; and
(d) repeating steps (a), (b), and (c), for a total of up to four product-use cycles per vaginal system, and achieving one or more of the following, as measured in a sample obtained from the female:
   i) an average segesterone acetate concentration in serum during the first quarterly product-use period of approximately 350 pmol/L to approximately 425 pmol/L;
   ii) an average ethinyl estradiol concentration in serum during the first quarterly product-use period of approximately 75 pmol/L to approximately 125 pmol/L
   iii) an average segesterone acetate concentration in serum during the second quarterly product-use period of approximately 275 pmol/L to approximately 350 pmol/L;
   iv) an average segesterone acetate concentration in serum during the third quarterly product-use period of approximately 225 to approximately 300 pmol/L of a third quarterly product-use period;
   v) an average segesterone acetate concentration in serum during the fourth quarterly product-use period of approximately 175 to approximately 250 pmol/L;
   vi) an average ethinyl estradiol concentration in serum during the second quarterly product-use period of approximately 75 pmol/L to approximately 100 pmol/L;
   vii) an average ethinyl estradiol concentration in serum during the third quarterly product-use period of approximately 55 to approximately 75 pmol/L; and
   viii) an average ethinyl estradiol concentration in serum during the fourth quarterly product-use period approximately 40 to approximately 60 pmol/L.

The concentration and AUC values described herein (regardless of use protocol) can be determined by withdrawing blood samples from a subject at various time points during use of the vaginal ring. In some embodiments, multiple blood samples can be taken and analyzed during the first day of insertion to capture, for example, Cₘₐₓ. In some embodiments, blood samples can be withdrawn on one or more additional days following the first day at intervals that are appropriate given the amount of time the ring system is intended to be retained in the vagina. In some embodiments, one or more blood samples can be taken and analyzed on the day the vaginal ring is removed from the vagina. Once the appropriate blood samples have been withdrawn, the various pharmacokinetic parameters described herein can be calculated using methods known to those of skill in the art.

Typically, in accordance with any methods disclosed herein, the vaginal system is self-inserted by the subject into the vagina for a product-use period and is removed for a removal period.

The vaginal system described herein can release therapeutic levels of SA and EE throughout the four quarterly product-use cycles or one 364-day product-use period. In certain embodiments, SA and EE can diffuse out of the vaginal system with release rates that vary over time. In certain embodiments, the daily in vitro release rates of SA and EE are higher during each initial 24-48 hours of use in a given product-use cycle, achieving a somewhat lower steady-state with continued use over subsequent days in each product-use cycle.

When used by a subject of reproductive potential, one system should be placed in the subject's vagina. Upon completion of a product-use period, the vaginal system should be removed for the removal period which, in typical embodiments, can be approximately 1 to 11 days. In some embodiments, the removal period can be approximately 3 to approximately 7 days. In certain embodiments, during the removal period, a withdrawal bleed can occur. Once removed, the vaginal system should be cleaned with mild soap and warm water, patted dry with a clean cloth towel or paper towel, and placed in its case for the duration of the removal period. At the end of the removal period, the vaginal system should be cleaned prior to being placed back in the vagina for another product-use period.

The vaginal system can be inserted using a variety of techniques. For example, the user can choose an insertion position that is comfortable, such as lying down, squatting, or standing. Typically, the sides of the vaginal system can be pressed together for insertion into a subject's vagina. When properly inserted, the vaginal system should be entirely in the vagina and behind the pelvic bone.

The system can be removed by hooking an index finger into the vaginal system inside the vagina and gently pulling the vaginal system.

After four quarterly product-use cycles or one 364-day product-use period, and because the vaginal system still contains both SA and EE, the system should be placed in a container, such as the container that may be provided with the vaginal system, and disposed of at a drug take-back location, if available. If a drug take-back location is not available, it should be discarded, in its container, in the trash.

### Beginning Use of the Vaginal System

If hormonal birth control is not being used (and if any copper IUD has been removed), the vaginal system should be started between days 2 and 5 of the menstrual period. If menstrual periods are not regular or if the system is started more than 5 days from the start of the menstrual period, a barrier method of birth control, such as a male condom or spermicide, should be used during sexual intercourse for the first 7 days that the vaginal system is used.

If changing from a combination hormonal birth control pill or patch or monthly disposable contraceptive vaginal ring to the vaginal system described herein, and the previous method has been used correctly and there is no possibility of pregnancy, a change to the system described herein can occur any day of the birth control cycle without the need for backup contraception. The vaginal system described herein cannot be started any later than the start date of the next birth control pill, the application date of the next patch, or the insertion date of the next monthly disposable contraceptive vaginal ring. No more than 7 hormone-free days should occur before starting the system.

If switching from a minipill, injection, implant, or an intrauterine system (i.e., Progestin-Only Method [Progestin-only pills (POP), Progestin Injection, Progestin Implant, Progestin Intrauterine System (IUS)] to the vaginal system described herein, and there are no contraindications to the use of EE, the switch can be made from a progestin-only method to the present system. If switching from progestin-only pills, the vaginal system should start at the time the next POP pill would have been taken. If switching from an injection, the vaginal system described herein should be started at the time of the next scheduled injection. If switching from an implant or an IUS, the system should be started at the time of implant or IUS removal. In all of these cases, an additional barrier method, such as a male condom or spermicide, should be used during sex for the first 7 days of use of the system.

If there are no contraindications to the use of EE, the vaginal system described herein can be initiated for contraception within the first 5 days following a complete first trimester abortion or miscarriage without additional back-up contraception. If more than 5 days have elapsed from the first trimester abortion or miscarriage, a non-hormonal birth control method, such as male condoms or spermicide, should be used while waiting for the next menstrual period to start. The vaginal system should then be started between days 2 and 5 of the menstrual period. If the system is started more than 5 days from the start of the menstrual period, a barrier method of birth control, such as a male condom or spermicide, should be used during sexual intercourse for the first 7 days that the system is used.

The vaginal system described herein should not be started earlier than 4 weeks (28 days) after a second trimester abortion or miscarriage (after the first 12 weeks of pregnancy) due to the increased risk of thromboembolism.

The vaginal system described herein should not be started sooner than 4 weeks postpartum and should only be started in subjects who choose not to breastfeed. Before 4 weeks postpartum there is an increased risk of thromboembolism. If the system is started 4 or more weeks after having a baby and a menstrual period has not started, another method of birth control should be used until the system has been used for 7 days in a row. The possibility of ovulation and conception occurring should be considered before initiating use of the vaginal system described herein.

Breastfeeding women should not use the vaginal system described herein. Females who are breastfeeding should use other birth control methods and not use the system until after weaning.

It has further been discovered that the vaginal system is compatible with condoms (male or female) made from natural rubber latex, polyurethane, and polyisoprene. That is, when the vaginal system is repeatedly exposed to a condom comprising the various polymers noted above over one or more of the four quarterly product-use cycles or over the 365-day product-use cycle, there is no concern that the system's efficacy will decrease because of this exposure, or conversely, that the system will have a negative impact on the condom. More specifically, contacting the vaginal system with one or more condoms over a product-use cycle will not cause or increase oxidative degradation of the vaginal system or the condom, cause or increase thermal degradation of the vaginal system or the condom, increase or decrease drug delivery rates from the vaginal system, cause or increase hydrolysis in the vaginal system or condom, or otherwise cause an unwanted reaction or side effect because of the interaction.

This compatibility is advantageous not only when using condoms as a secondary/backup form of birth control when needed, but also when using condoms to prevent the transmission of sexually transmitted infections because the vaginal system described herein does not protect against HIV-infection (AIDS) and other sexually transmitted infections.

### Considerations for Use of the Vaginal System

The vaginal system described herein has not been adequately evaluated in females with a body mass index of >29 kg/m2. This subpopulation was excluded from the clinical trials after two venous thromboembolisms (VTEs) occurred in this group. Higher body weight associates with lower systemic exposure of SA and EE. In a PK study conducted in 18 females with BMI <25 (16.89-24.34) kg/m and 21 females with BMI >25 (25.15-37.46) kg/m, up to 16% and 33% decreases in the systemic exposure (AUC 0-21 day) of SA and EE, respectively, were observed between the two BMI groups.

The vaginal system described herein is contraindicated for females with a high risk of arterial or venous thrombotic diseases. Examples include females over the age of 35 who smoke, females who have or have a history of deep vein thrombosis or pulmonary embolism, females who have cerebrovascular disease, females who have coronary artery disease. In addition, the vaginal system is contraindicated in females who have thrombogenic valvular or thrombogenic rhythm disease of the heart (for example, subacute bacterial endocarditis with valvular disease, or atrial fibrillation). The vaginal system described herein is also contraindicated in females who have inherited or acquired hypercoagulopathies. The system should be discontinued immediately if there is unexplained loss of vision, proptosis, diplopia, papilledema, or retinal vascular lesions and evaluate for retinal vein thrombosis.

The vaginal system described herein is contraindicated in females who have or have a history of breast cancer or other estrogen-or progestin-sensitive cancer, for females who have liver tumors, acute hepatitis, or severe (decompensated) cirrhosis, for females who have undiagnosed abnormal uterine bleeding, for females who have a hypersensitivity to any of the components of the system and for females who use Hepatitis C drug combinations containing ombitasvir/paritaprevir/ritonavir, with or without dasabuvir. The system can be restarted 2 weeks following completion of this regimen. Use of the vaginal system described herein should be stopped if a thrombotic or thromboembolic event occurs. The system should be stopped at least 4 weeks before and through 2 weeks after major surgery or other surgeries known to have an elevated risk of VTE

Cardiovascular risk factors should be considered before initiating in all females, particularly those over 35 years. The system should not be used by females with uncontrolled hypertension or hypertension with vascular disease. If used in females with well-controlled hypertension, blood pressure should be monitored and use of the system discontinued if blood pressure rises significantly.

The vaginal system described herein is contraindicated in females with acute hepatitis or severe (decompensated) cirrhosis of the liver. It should be discontinued if jaundice occurs. Acute liver test abnormalities may necessitate the discontinuation use until the liver tests return to normal and system causation has been excluded.

Glucose should be monitored in prediabetic and diabetic females taking the vaginal system described herein. An alternate contraceptive method should be considered for females with uncontrolled dyslipidemias. The system is contraindicated for females with diabetes mellitus who are over age 35, females who have diabetes mellitus with hypertension or vascular disease, who have other end-organ damage, or who have diabetes mellitus of >20 years duration. Females with hypertriglyceridemia, or a family history thereof, may be at an increased risk of pancreatitis when using the system.

Significant change in headaches should be evaluated and use of the system discontinued if indicated. The vaginal system is contraindicated in females who have headaches with focal neurological symptoms, who have migraine headaches with aura, or who are over age 35 with any migraine headaches.

The vaginal system described herein may cause irregular bleeding or amenorrhea. Bleeding/spotting that occurs during the dose-free week when the vaginal system is out is considered “scheduled” bleeding. Bleeding and/or spotting that occurs at any time while the vaginal system is inserted is considered “unscheduled” bleeding/spotting. If these persist, other causes should evaluated.

The most common adverse reactions (>5%) are headache/migraine, nausea/vomiting, vulvovaginal mycotic infection/candidiasis, abdominal pain lower/upper, dysmenorrhea, vaginal discharge, urinary tract infection, breast tenderness/pain/discomfort, bleeding irregularities including metrorrhagia, diarrhea, genital pruritus. Serious adverse reactions occurring in ≥2 subjects were: VTEs (deep venous thrombosis, cerebral vein thrombosis, pulmonary embolism); psychiatric events; drug hypersensitivity reactions; and spontaneous abortions.

Drugs or herbal products that induce certain enzymes, including CYP3A4, may decrease the effectiveness of the system or increase breakthrough bleeding. Examples of incompatible drugs and herbal products include, but are not limited to, aprepitant, barbiturates, bosentan, carbamazepine, efavirenz, felbamate, griseofulvin, oxcarbazepine, phenytoin, rifampin, rifabutin, rufinamide, topiramate, products containing St. John's wart, and certain protease inhibitors. Back-up or alternative method of contraception should be used when enzyme inducers are used with the system. Continue back-up contraception for 28 days after discontinuing the enzyme inducer to maintain contraceptive reliability.

Co-administration of atorvastatin or rosuvastatin and certain CHCs containing EE increase systemic exposure of EE by approximately 20-25%. Ascorbic acid and acetaminophen may increase plasma EE concentrations, possibly by inhibition of conjugation. CYP3A4 inhibitors such as itraconazole, voriconazole, fluconazole, grapefruit juice, or ketoconazole may increase systemic exposure of the estrogen and/or progestin component of the system.

Significant decreases in systemic exposure of estrogen and/or progestin have been noted when CHCs are co-administered with some HIV protease inhibitors (e.g., nelfinavir, ritonavir, darunavir/ritonavir, (fos)amprenavir/ritonavir, lopinavir/ritonavir, and tipranavir/ritonavir), some HCV protease inhibitors (e.g., boceprevir and telaprevir), and some non-nucleoside reverse transcriptase inhibitors (e.g., nevirapine). In contrast, significant increases in systemic exposure of estrogen and/or progestin have been noted when CHCs are co-administered with certain other HIV protease inhibitors (e.g., indinavir and atazanavir/ritonavir) and with other non-nucleoside reverse transcriptase inhibitors (e.g., etravirine).

Concomitant use of CHCs with lamotrigine may significantly decrease systemic exposure of lamotrigine due to induction of lamotrigine glucuronidation. Decreased systemic exposure of lamotrigine may reduce seizure control. Dose adjustment for lamotrigine may be necessary. Product labeling for lamotrigine should be consulted.

Concomitant use of CHCs with thyroid hormone replacement therapy or corticosteroid replacement therapy may increase systemic exposure of thyroid-binding and cortisol-binding globulin. The dose of replacement thyroid hormone or cortisol therapy may need to be increased. Approved product labeling for the therapy in use should be consulted.

Concomitant use of CHCs may decrease systemic exposure of acetaminophen, morphine, salicylic acid, and temazepam. Concomitant use with ethinyl estradiol containing CHCs may increase systemic exposure of other drugs (e.g., cyclosporine, prednisolone, theophylline, tizanidine, and voriconazole). The dosage of drugs that can be affected by this interaction may need to be increased or decreased. The approved product labeling for the concomitantly used drug should be consulted.

In a drug-drug interaction study with the vaginal system described herein and the concurrent use of three different formulations of vaginal miconazole, the use of water-based vaginal miconazole cream resulted in no change in exposure to EE or SA from the vaginal system. However, the use of either the 1 day or the 3-day oil-based miconazole suppository was associated with an overall increase in exposure up to 67% for EE and 32% for SA. Considering the potential long-term effect on vaginal system performance, concurrent use of oil-based vaginal suppositories should not occur with the system's use. If there is a need to treat a vaginal condition, water-based vaginal cream or oral therapy may be used concurrently with the vaginal system.

Water-based vaginal lubricants have no effect on the vaginal system described herein; however, oil-based (including silicone-based) vaginal lubricants will alter the vaginal system and/or exposure to EE and SA and should not be used.

The effect of tampon use on the systemic exposure of SA and EE from the vaginal system described herein has not been studied.

The use of contraceptive steroids may influence the results of certain laboratory tests, such as coagulation factors, lipids, glucose tolerance, and binding proteins.

Females with a history of depression should be monitored and the system should be discontinued if depression recurs to a serious degree.

The estrogen component of the vaginal system described herein may raise the serum concentrations of thyroxine-binding globulin, sex hormone-binding globulin, and cortisol-binding globulin. The dose of replacement thyroid hormone or cortisol therapy may need to be increased.

In females with hereditary angioedema, exogenous estrogens may induce or exacerbate symptoms of angioedema.

Chloasma may occur with use of the vaginal system described herein, especially in females with a history of chloasma gravidarum. Females who tend to develop chloasma should avoid exposure to the sun or ultraviolet radiation while using the system.

Cases of toxic shock syndrome (TSS) have been reported by vaginal ring users. TSS has been associated with tampons and certain barrier contraceptives, and in some TSS cases ring users were also using tampons. Causal relationship between the use of a vaginal ring and TSS has not been established. No cases of TSS occurred in clinical trials with use of the vaginal system described herein. If a female exhibits signs or symptoms of TSS, the possibility of this diagnosis should be considered and the vaginal system should be removed. Appropriate medical evaluation and treatment should be initiated.

Some females are aware of the vaginal system on occasion during days of use or during sex, and partners may feel the vaginal system during sex.

Epidemiologic studies and meta-analyses have not found an increased risk of genital or nongenital birth defects (including cardiac anomalies and limb-reduction defects) following exposure to CHCs before conception or during early pregnancy. In the U.S. general population, the estimated background risk of major birth defects and miscarriage in clinically recognized pregnancies is 2-4% and 15-20%, respectively. The vaginal system described herein should be discontinued if pregnancy occurs, because there is no reason to use CHCs during pregnancy. No studies have been conducted of the use of the system in pregnant females.

No studies were conducted in subjects with renal impairment; the vaginal system described herein is not recommended in subjects with renal impairment.

There have been no reports of serious ill effects from overdose of CHCs. Overdosage may cause withdrawal bleeding in females and nausea. In case of suspected overdose, all vaginal systems should be removed and symptomatic treatment given.

### Carcinogenesis

In a 2-year carcinogenicity study in rats with subdermal implants releasing 40, 100, and 200 μg segesterone acetate per day (approximately 17-86 times the daily dose of segesterone acetate in females using the system based on body surface area), no drug-related increase in tumor incidence was observed. In a 2-year intravaginal carcinogenicity study in mice, segesterone acetate gel produced an increased incidence of adenocarcinoma and lobular hyperplasia in the breast at a dose of 30 mg/kg/day, approximately 10 times the systemic exposure of segesterone acetate per day in females using the system described herein based on AUC. A dose of 10 mg/kg/day in the mouse, approximately 3 times the systemic exposure of segesterone acetate per day based on AUC, did not result in carcinogenic findings. Long-term continuous administration of natural and synthetic estrogens in certain animal species increases the frequency of carcinomas of the breast, uterus, cervix, vagina, testis, and liver.

### Mutagenesis

Segesterone acetate was neither mutagenic nor clastogenic in the Ames/Salmonella reverse mutation assay, the chromosomal aberration assay in Chinese hamster ovary cells, or in the in vivo mouse micronucleus test.

### Impairment of Fertility

A return to fertility study was conducted with segesterone acetate in rats, using subdermal implants releasing a dose approximately 25 times the anticipated daily vaginal human dose (based on body surface area). Three months of treatment with segesterone acetate suppressed fertility, but 7 weeks after cessation of treatment, there were no adverse effects on ovulation or resulting litter parameters.

Resumption of fertility after discontinuation of the vaginal system described herein is expected. All women followed for return of fertility experienced a return of fertility by 6 months after discontinuing use of the system.

### Vaginal System Structure

In general, the vaginal system described herein is an appropriately sized and shaped structure suitable for insertion to the vagina. The system typically comprises at least two parts: a ring body and one or more cores. The cores can be shaped in a way that is suitable for containment within the ring. The ring body is typically prepared from one or more polymeric materials, such as one or more silicone elastomers, and is generally adapted to receive, or to be coextruded with, at least one drug-containing core. The at least one drug-containing core can be prepared from the same or different polymeric materials as the ring body. The core can contain active ingredients, such as EE, SA, or a combination thereof, dissolved, dispersed (i.e., as a solid), or dissolved and dispersed throughout the at least one core. When combined, the ring body and at least one core provide the active ingredients to the user via a release rate sufficient to provide efficacious birth control over four quarterly product-use cycles or a 365-day product-use cycle.

While the ring body itself is typically manufactured without the addition of active agents, such as SA or EE, in certain embodiments, the ring body can be prepared such that it includes SA, EE, or both within the ring body in addition to or instead of within the cores. It is understood, however, that when the ring body is manufactured without active agents, either active agent or both active agents can diffuse from the cores into the ring body before the first product-use cycle.

In certain embodiments, the vaginal system of the present disclosure is ring-shaped, having an overall (exterior) diameter, an interior diameter, and a cross-sectional diameter. In some embodiments, the ring has an overall (exterior) diameter of from approximately 40 mm to approximately 70 mm. In other embodiments, the ring has an overall diameter of from approximately 45 mm to approximately 65 mm. In other embodiments, the ring an overall diameter of from approximately 50 mm to approximately 60 mm. In other embodiments, the ring has an overall diameter of from approximately 53 mm to approximately 59 mm. In some embodiments, the ring has an overall diameter of approximately 56 mm.

In certain embodiments, the ring has an interior diameter of from approximately 25 mm to approximately 55 mm. In other embodiments, the ring has an interior diameter of from approximately 30 mm to approximately 50 mm. In other embodiments, the ring has an interior diameter of from approximately 35 mm to approximately 45 mm. In some embodiments, the ring has an interior diameter of approximately 40 mm.

In certain embodiments, the vaginal system of the present disclosure is ring-shaped and has a cross-sectional diameter of from approximately 3 mm to approximately 10 mm. In other embodiments, the ring has a cross-sectional diameter of from approximately 3.5 mm to approximately 9.5 mm. In other embodiments, the ring has a cross-sectional diameter of from approximately 4 mm to approximately 9 mm. In other embodiments, the ring has a cross-sectional diameter of from approximately 5 to approximately 9 mm. In other embodiments, the ring has a cross-sectional diameter of from approximately 6 to approximately 9 mm. In other embodiments, the ring has a cross-sectional diameter of from approximately 7 to approximately 9 mm. In other embodiments, the ring has a cross-sectional diameter of from approximately 8 to approximately 9 mm. In some embodiments, the ring has a cross-sectional diameter of approximately 8.4 mm.

Sizing of the vaginal system is an important component in system design. As the system is inserted into a woman's vagina, the vaginal system can neither be too large nor too small to make insertion and/or retrieval more difficult. Similarly, the cross-sectional diameter of the vaginal system is another design component that can be tailored to provide optimal drug delivery and comfort so that the system is not considered aesthetically “bulky” or sensed within the vagina by the woman.

The vaginal system typically adopts the shape of the ring body such that, and by way of example only, when the ring body is ring-shaped, the vaginal system is ring shaped. Although the vaginal system can be ring-shaped, in some embodiments, the vaginal system can be an elliptic or oblong torus, a bohemian dome, lemon shaped, an “eight surface,” an ellipsoid, a heart surface, a sphere, a spheroid, or any other shape suitable for insertion into the subject's vagina. In some embodiments, the vaginal system can be circular or spherical. In some embodiments, the vaginal system can be in the shape of a polygon. In some embodiments, the vaginal system can be rectangular, triangular, hexagonal, petagonal, rhomboid, triangular prism, or spherical. Any shape that is appropriate for insertion into a vagina to provide maximal comfort to the user without deviating from the teaching provided in this disclosure can be selected or used.

Regardless of its shape, and in certain embodiments, the vaginal system comprises one or more channels adapted to receive at least one core. When the ring body comprises more than one core, the channels adapted to receive the cores can be on opposing sides of the ring body. In other embodiments, the channels adapted to receive the cores are in closer to proximity to each other. In some embodiments, the channels adapted to receive the cores are adjacent to each other within the ring body. In some embodiments, the channels adapted to receive the cores abut one another. In some embodiments, the channels adapted to receive the cores are both situated in the same half of the ring body.

The release rate of the agent or agents contained within cores is affected by the length of the path the agent or agents must diffuse through to exit the system into the subject. For example, a shorter diffusion path within the ring body can provide an increased release rate, while a longer diffusion path can provide a decreased release rate. As such, the amount of active agent or agents contained within the cores must be balanced against diffusion path length, among other considerations. In some embodiments, channels adapted to receive the cores have a length of from approximately 10 mm to approximately 40 mm. In other embodiments, channels adapted to receive the cores have a length of from approximately 15 mm to approximately 35 mm. In other embodiments, channels adapted to receive the cores have a length of from approximately 20 mm to approximately 35 mm. In other embodiments, channels adapted to receive the cores have a length of from approximately 25 mm to approximately 30 mm. In other embodiments, the channels adapted to receive the cores have a length of approximately 27 mm.

The channel or channels adapted to receive the at least one core can be any appropriate shape. For example, in some embodiments, the channel or channels adapted to receive the core(s) can be a bore, such as a cylindrical bore adapted to receive an appropriately shaped cylindrical or spherical core. In other embodiments, the channel or channels can be adapted to receive a core or cores shaped like a rectangular prism, including for example a square prism, or a core or cores shaped like a cone, a triangular prism, a triangular pyramid, a rectangular pyramid, a pentagonal prism, a hexagonal prism, a heptagonal prism, or any other three-dimensional shape suitable for manufacture. In some embodiments, the channel or channels can be adapted to receive a core or cores that are disc shaped. In certain embodiments, the channel or channels can be adapted to receive a cylindrical core or core shaped like a rectangular prism.

In some embodiments, the channel or channels adapted to receive the at least one core are adapted to receive a cylindrical core having a diameter of from approximately 1 mm to approximately 7 mm. In other embodiments, the channel or channels adapted to receive the at least one core are adapted to receive a cylindrical core having a diameter of from approximately 2 mm to approximately 6 mm. In other embodiments, the channel or channels adapted to receive the at least one core are adapted to receive a cylindrical core having a diameter of from approximately 2 mm to approximately 5 mm. In other embodiments, the channel or channels adapted to receive the at least one core are adapted to receive a cylindrical core having a diameter of from approximately 2 mm to approximately 4 mm. In other embodiments, the channel or channels adapted to receive the at least one core are adapted to receive a cylindrical core having a diameter of approximately 3 mm.

In some embodiments, the cores are coextruded with the elastomers of the ring body. In other embodiments, the cores can be extruded or formed by injection molding, allowed to cure, and the ring body elastomers extruded in a manner to encase the cores.

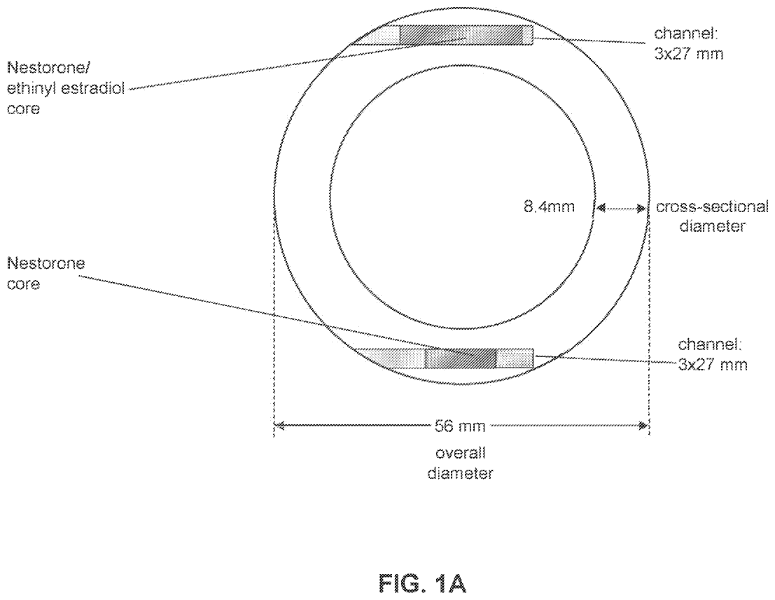

In certain embodiments, the vaginal system of the present disclosure is ring-shaped and is 56 mm in overall diameter and has a cross-sectional diameter of 8.4 mm. In some embodiments, it contains two channels, each of which is approximately 3 mm in diameter and approximately 27 mm in length, each of which is adapted to receive an appropriately sized and shaped steroid-containing core. An example of such an embodiment is shown in FIGS. 1A and 1B.

It is understood that in certain embodiments, the channels are formed in the ring at the time the ring body is prepared, either by injection molding or extrusion. In other embodiments, the channels are formed about the cores during extrusion or injection molding of the ring body.

### Cores

In certain embodiments, the vaginal system contains from approximately 50 to approximately 150 mg of SA and from approximately 5 to approximately 35 mg of EE which are distributed throughout one or more cores. In certain embodiments, the vaginal system contains from approximately 75 to approximately 125 mg of SA and from approximately 10 to approximately 25 mg of EE which are distributed throughout one or more cores. In certain embodiments, the vaginal system contains from approximately 90 to approximately 115 mg of SA and from approximately 15 to approximately 20 mg of EE which are distributed throughout one or more cores. In some embodiments, the vaginal system contains approximately 103 mg of SA and approximately 17.4 mg of EE which are distributed throughout one or more cores. In certain embodiments, the vaginal system contains from approximately 50 to approximately 150 mg of SA and from approximately 5 to approximately 35 mg of EE which are distributed throughout a single core. In certain embodiments, the vaginal system contains from approximately 75 to approximately 125 mg of SA and from approximately 10 to approximately 25 mg of EE which are distributed throughout a single core. In certain embodiments, the vaginal system contains from approximately 90 to approximately 115 mg of SA and from approximately 15 to approximately 20 mg of EE which are distributed throughout a single core. In some embodiments, the vaginal system contains approximately 103 mg of SA and approximately 17.4 mg of EE which are distributed throughout a single core. In certain embodiments, the vaginal system contains from approximately 50 to approximately 150 mg of SA and from approximately 5 to approximately 35 mg of EE which are distributed throughout multiple cores. In certain embodiments, the vaginal system contains from approximately 75 to approximately 125 mg of SA and from approximately 10 to approximately 25 mg of EE which are distributed throughout multiple cores. In certain embodiments, the vaginal system contains from approximately 90 to approximately 115 mg of SA and from approximately 15 to approximately 20 mg of EE which are distributed throughout multiple cores. In some embodiments, the vaginal system contains approximately 103 mg of SA and approximately 17.4 mg of EE which are distributed throughout multiple cores. In some embodiments, the SA is distributed throughout one core and the EE is distributed throughout a separate core. In some embodiments, the SA is distributed throughout one core and the EE is distributed throughout multiple cores. In some embodiments, the SA is distributed throughout multiple cores and the EE is distributed throughout a separate core. In certain embodiments, the vaginal system contains from approximately 50 to approximately 150 mg of SA and from approximately 5 to approximately 35 mg of EE which are distributed in two or more cores, i.e. each core in the system contains both SA and EE. In certain embodiments, the vaginal system contains from approximately 75 to approximately 125 mg of SA and from approximately 10 to approximately 25 mg of EE which are distributed in two or more cores, i.e., each core in the system contains both SA and EE. In certain embodiments, the vaginal system contains from approximately 90 to approximately 115 mg of SA and from approximately 15 to approximately 25 mg of EE which are distributed in two or more cores, i.e., each core in the system contains both SA and EE. In yet another embodiment, the vaginal system contains approximately 103 mg of SA and approximately 17.4 mg of EE which are each distributed in two or more cores, i.e., each core in the system contains both SA and EE.

In a particular embodiment, the vaginal system comprises two cores that collectively contain 103 mg of SA and 17.4 mg of EE. In one such embodiment, one core contains 17.4 mg of EE and a portion of the SA drug load. The other core, in this embodiment, contains the remainder of the SA drug load. Of course, both cores can contain both actives. In some embodiments, the EE drug load is contained in a first core and the SA drug load is split amongst two or more cores.

In some embodiments, the vaginal system contains approximately 103 mg of SA distributed throughout two cores and approximately 17.4 mg of EE distributed throughout only one of the two cores, such that one core contains only SA, while the other core contains both SA and EE. In certain embodiments, the SA is distributed between the two cores in a ratio from approximately 90:10 to approximately 10:90. In other embodiments, the SA is distributed between the two cores in a ratio from approximately 80:20 to approximately 20:80. In other embodiments, the SA is distributed between the two cores in a ratio from approximately 70:30 to approximately 30:70. In other embodiments, the SA is distributed between the two cores in a ratio from approximately 60:40 to approximately 40:60. In other embodiments, the SA is distributed between the two cores in approximately a 50:50 ratio. In certain embodiments, the SA is distributed between the two cores in a ratio of from approximately 55:45 to approximately 45:55. In some embodiments, the SA is distributed between the two cores in approximately a 55:45 ratio.

In typical embodiments, the EE is present in one core and is substantially or completely absent from the second core. In other embodiments, however, the EE is distributed between the two cores in a ratio from approximately 99:1 to approximately 1:99. In other embodiments, the EE is distributed between the two cores in a ratio from approximately 95:5 to approximately 5:95. In certain embodiments, the EE is distributed between the two cores in a ratio from approximately 90:10 to approximately 10:90. In other embodiments, the EE is distributed between the two cores in a ratio from approximately 80:20 to approximately 20:80. In other embodiments, the EE is distributed between the two cores in a ratio from approximately 70:30 to approximately 30:70. In other embodiments, the EE is distributed between the two cores in a ratio from approximately 60:40 to approximately 40:60. In other embodiments, the EE is distributed between the two cores in approximately a 50:50 ratio.

In some embodiments, the vaginal system comprises a first core which contains from approximately 40% to approximately 60% SA by mass. In some embodiments, the first core contains from approximately 45% to approximately 55% SA by mass. In certain embodiments, the first core contains approximately 50% SA by mass.

In some embodiments, the first core is from approximately 1 mm to approximately 5 mm in diameter. In some embodiments, the first core is from approximately 2 mm to approximately 4 mm in diameter. In some embodiments, the first core is approximately 3 mm in diameter. In certain embodiments, the first core is from approximately 9 mm to approximately 13 mm in length. In certain embodiments, the first core is from approximately 10 mm to approximately 12 mm in length. In some embodiments, the first core is approximately 11 mm in length.

In some embodiments, the vaginal system comprises a second core which contains from approximately 30% to approximately 50% SA by mass. In some embodiments, the second core contains from approximately 35% to approximately 45% SA by mass. In some embodiments, the second core contains approximately 40% SA by mass. In some embodiments, the second core also contains from approximately 5% to approximately 20% EE by mass. In some embodiments, the second core contains from approximately 10% to approximately 14% EE by mass. In some embodiments, the second core contains approximately 12% EE by mass. In some embodiments, the second core is from approximately 1 mm to approximately 5 mm in diameter. In some embodiments, the second core is from approximately 2 mm to approximately 4 mm in diameter. In some embodiments, the second core is approximately 3 mm in diameter. In some embodiments, the second core is from approximately 16 mm to approximately 20 mm in length. In some embodiments, the second core is from approximately 17 mm to approximately 19 mm in length. In some embodiments, the second core is approximately 18 mm in length.

In certain embodiments, the vaginal system cores comprise one or more polymers. In certain embodiments, the vaginal system cores comprise one or more polymers selected from a polystyrene, a thermoplastic polymer (including, but not limited to, poly(methyl methacrylate), acrylonitrile butadiene styrene, nylon, polylactic acid, polybenimidazole, polycarbonate, polyether sulfone, polyoxymethylene, polyetherketone, polyetherimide, polyethylene, polyphenylene oxide, polyphenylene sulfide, polypropylene, polystyrene, polyvinyl chloride, polyvinylidene floride, and teflon), and elastomers (including, but not limited to natural and synthetic polyisoprene, polybutadiene, chloroprene, butyl rubber (including halogenated derivatives thereof), styrene-butadiene, nitrile rubber (including halogenated derivatives thereof), ethylene/propylene rubbers (including both melt blends and reactor blends (block copolymers) of ethylene and propyelene), epichlorohydrin rubber, polyacrylic rubber, a silicone elastomer, fluorosilicone rubber, a fluoroelastomer (e.g. VITON, TECNOFLON, FLUOREL, AFAS, and DAI-EL), a perfluoroelastomer, a polyether block amide, chlorosulfonated polyethylene, ethylene vinylacetate (“EVA”)). In some embodiments, the cores comprise EVA. In some embodiments, the cores comprise one or more elastomers, wherein the elastomers are silicone elastomers. In some embodiments, the cores comprise a mixture of silicone and other elastomers. In some embodiments, the vaginal system cores comprise a single silicone elastomer. In other embodiments, the vaginal system cores are comprised of multiple silicone elastomers. In some embodiments, one or more of the cores comprises a single silicone elastomer and one or more of the cores comprises multiple silicone elastomers.

In some embodiments, the silicone elastomers comprise one or more agents to increase viscosity. In some embodiments, the one or more agents to increase viscosity can be diatomaceous earth, cellulose, talc, and/or silica (e.g., fumed silica or colloidal silica). In some embodiments, the agent to increase viscosity is diatomaceous earth.

In some embodiments, the vaginal system described herein comprises condensation cure silicone elastomeric cores. In some embodiments, the vaginal system comprises addition-cure silicone elastomeric cores. In some embodiments, the vaginal system comprises one or more condensation cure silicone elastomeric cores and one or more condensation cure silicone elastomeric cores.

In some embodiments, the vaginal system comprises a first core which comprises one or more condensation cure silicone elastomers. In some embodiments, the first core comprises two condensation cure silicone elastomers. In some embodiments, one or both of these condensation cure silicone elastomers can contain one or more agents to increase its viscosity. In some embodiments, the one or more agents to increase viscosity can be diatomaceous earth, cellulose, talc, and/or silica (e.g., fumed silica or colloidal silica). In some embodiments, the agent to increase viscosity is diatomaceous earth.

In some embodiments, the condensation cure silicone elastomer can be NuSil™ MED-6381. In certain embodiments, this condensation-cure silicone elastomer can be prepared from three components, “Part A,” “Part B,” and a tin catalyst. In some embodiments, Part A comprises >90% hydroxyl-terminated dimethylsiloxanes and dimethylsilicones (CAS No. 70131-67-8). In some embodiments, Part B comprises >90% tetrapropyl orthosilicate (CAS No. 682-01-9). In certain embodiments, the tin catalyst can be di-n-butylbutoxychlorotin, dibutyldiacetoxytin, dibutyltin dilaurate, dimethyldineodecanoatetin, dioctyldilauryltin, tetramethyltin, dioctylbis(2-ethylhexylmaleate)tin, or stannous octanoate. In some embodiments, the tin catalyst is stannous octanoate or dibutyltin dilaurate.

In certain embodiments, the condensation cure silicone elastomer can be NuSil™ MED-6382. In certain embodiments, this condensation-cure silicone elastomer can be prepared from two components, “Part A” and a tin catalyst. In some embodiments, Part A comprises siloxanes, silicones, and <1% amorphous, fumed, crystalline free silica (CAS No. 112945-52-5). In certain embodiments, the tin catalyst can be di-n-butylbutoxychlorotin, dibutyldiacetoxytin, dibutyltin dilaurate, dimethyldineodecanoatetin, dioctyldilauryltin, tetramethyltin, dioctylbis(2-ethylhexylmaleate)tin, or stannous octanoate. In some embodiments, the tin catalyst is stannous octanoate or dibutyltin dilaurate.

In further embodiments, the condensation cure silicone elastomer can be NuSil™ MED-6603 (formerly known as DDU-4352). In certain embodiments, this condensation-cure silicone elastomer can be prepared from two components, “Part A” and a tin catalyst. In some embodiments, Part A comprises siloxanes and silicones. In certain embodiments, the tin catalyst can be di-n-butylbutoxychlorotin, dibutyldiacetoxytin, dibutyltin dilaurate, dimethyldineodecanoatetin, dioctyldilauryltin, tetramethyltin, dioctylbis(2-ethylhexylmaleate)tin, or stannous octanoate. In some embodiments, the tin catalyst is stannous octanoate or dibutyltin dilaurate.

In further embodiments, the condensation cure silicone elastomer can be NuSil™ MED3-6603. In certain embodiments, this condensation-cure silicone elastomer can be prepared from three components, “Part A,” “Part B,” and a tin catalyst. In some embodiments, Part A comprises polydimethylsiloxane backbone. In some embodiments, Part B comprises the cross-linking agent. In certain embodiments, the tin catalyst can be di-n-butylbutoxychlorotin, dibutyldiacetoxytin, dibutyltin dilaurate, dimethyldineodecanoatetin, dioctyldilauryltin, tetramethyltin, dioctylbis(2-ethylhexylmaleate)tin, or stannous octanoate. In some embodiments, the tin catalyst is stannous octanoate or dibutyltin dilaurate.

In some embodiments, the condensation cure silicone elastomer can be NuSil™ MED-6385. In certain embodiments, this condensation-cure silicone elastomer can be prepared from two components, “Part A” and a tin catalyst. In some embodiments, Part A comprises dimethylsiloxanes, dimethylsilicones (CAS No. 70131-67-8), 20-25% diatomaceous earth (CAS No. 68855-54-9), <5% silicic acid, tetrapropyl ester (CAS No. 682-01-9), and <1% amorphous, fumed, crystalline free silica (CAS No. 112945-52-5). In certain embodiments, the tin catalyst can be di-n-butylbutoxychlorotin, dibutyldiacetoxytin, dibutyltin dilaurate, dimethyldineodecanoatetin, di-octyldilauryltin, tetramethyltin, dioctylbis(2-ethylhexylmaleate)tin, or stannous octanoate. In some embodiments, the tin catalyst is stannous octanoate or dibutyltin dilaurate.

In still further embodiments, the condensation cure silicone elastomer can be NuSil™ MED3-6385. In certain embodiments, this condensation-cure silicone elastomer can be prepared from three components, “Part A,” “Part B,” and a tin catalyst. In some embodiments, Part A comprises polydimethylsiloxane polymer backbone and diatomaceous earth. In some embodiments, Part B comprises the cross-linking agent. In certain embodiments, the tin catalyst can be di-n-butylbutoxychlorotin, dibutyldiacetoxytin, dibutyltin dilaurate, dimethyldineodecanoatetin, di-octyldilauryltin, tetramethyltin, dioctylbis(2-ethylhexylmaleate)tin, or stannous octanoate. In some embodiments, the tin catalyst is stannous octanoate or dibutyltin dilaurate.

Each of these polymers is commercially available and is referenced in one or more drug master files.

In certain embodiments, the first silicone elastomer of the first core is NuSil™ MED-6385. In some embodiments, the second silicone elastomer of the first core is NuSil™ MED-6603 (formerly known as DDU-4352). In some embodiments, the tin catalyst is dibutyltin dilaurate.

In certain embodiments, the first core comprises SA homogeneously dispersed or distributed in a silicone elastomer comprising at least two condensation cure silicone elastomers. In certain embodiments, the core can be prepared by combining the first silicone elastomer and the second silicone elastomer, adding the SA, and blending the resulting mixture. In certain embodiments, the SA can be added in batches. After sufficient mixing, a curing agent can be added, and the resulting mixture can be blended further. In some embodiments, the curing agent can be a tin catalyst. In certain embodiments, the tin catalyst can be di-n-butylbutoxychlorotin, dibutyldiacetoxytin, dibutyltin dilaurate, dimethyldineodecanoatetin, di-octyldilauryltin, tetramethyltin, dioctylbis(2-ethylhexylmaleate)tin, or stannous octanoate. In some embodiments, the tin catalyst is stannous octanoate or dibutyltin dilaurate. In some embodiments, the curing agent can be dibutyltin dilaurate. In some embodiments, the curing agent is NuSil™ MED-6603 Part B. In some embodiments, the blended mixture, also referred to as a pre-core mixture, can be shaped into a string and be subjected to curing conditions.

In certain embodiments, the pre-core mixture can be shaped into strings by injection molding. In some embodiments, the pre-core mixture can be shaped into strings by extrusion. In certain embodiments, the strings can be cured at a temperature of from approximately room temperature to approximately 140° ° C. In some embodiments, the strings can be cured at a temperature of from approximately 40° C. to approximately 135° C. In certain embodiments, the strings can be cured at a temperature of from approximately 50° C. to approximately 130° C. In some embodiments, the strings can be cured at a temperature of from approximately 55° C. to approximately 125° C. In some embodiments, the strings can be cured at a temperature of from approximately 60° C. to approximately 120° C.

In some embodiments, the amount of time that the strings are cured increases with decreasing curing temperature. In certain embodiments, the strings can be cured for from approximately 10 minutes to approximately 70 minutes. In certain embodiments, the strings can be cured for from approximately 20 minutes to approximately 60 minutes. In some embodiments, the strings can be cured for from approximately 25 minutes to approximately 50 minutes. In some embodiments, the strings can be cured for from approximately 30 minutes to approximately 45 minutes. In some embodiments, the strings can be cured for approximately 30 minutes. In some embodiments, the strings can be cured for approximately 45 minutes. In some embodiments, the strings can be cured at approximately 120° C. for approximately 30 minutes. In some embodiments, the strings can be cured at approximately 60° C. for approximately 45 minutes.

In some embodiments, the cured product can be post-cured at room temperature for at least 2 days. In some embodiments, the cured product can be post-cured at room temperature for at least 3 days. In some embodiments, the cured product can be post-cured at room temperature for at least 4 days. In some embodiments, the cured product can be post-cured at room temperature for at least 5 days. In some embodiments, the cured product can be post-cured at room temperature for at least 6 days. In some embodiments, the cured product can be post-cured at room temperature for at least 7 days. In some embodiments, the cured product is post-cured at room temperature for at least 8 days. In some embodiments, the cured product can be post-cured at room temperature for at least 9 days. In some embodiments, the cured product can be post-cured at room temperature for at least 10 days.

In certain embodiments, the strings can be cut after post-curing to provide cores suitable for providing the desired SA and EE release rates as disclosed herein. As core length and diameter can affect the release rate of the agents, the amount of a particular agent added to a particular core needs to be balanced against the length and diameter of that core to ensure that the release rates disclosed herein are attained. In some embodiments, the strings can be cut to a length from approximately 8 mm to approximately 14 mm. In some embodiments, the strings can be cut to a length from approximately 9 mm to approximately 13 mm. In some embodiments, the strings can be cut to a length from approximately 10 mm to approximately 12 mm. In some embodiments, the strings can be cut to a length of approximately 11 mm. In some embodiments, the weight of the first core can be from approximately 70 to approximately 120 mg. In some embodiments, the weight of the first core can be from approximately 80 to approximately 100 mg. In some embodiments, the weight of the first core can be from approximately 85 mg to approximately 95 mg. In some embodiments, the weight of the first core is approximately 90 mg.

In certain embodiments, the first core can contain from approximately 25 mg to approximately 75 mg of SA. In some embodiments, the first core can contain from approximately 35 mg to approximately 65 mg of SA In some embodiments, the first core can contain from approximately 40 mg to approximately 50 mg of SA. In some embodiments, the first core contains approximately 45 mg of SA or from 43 mg to 47 mg of SA.

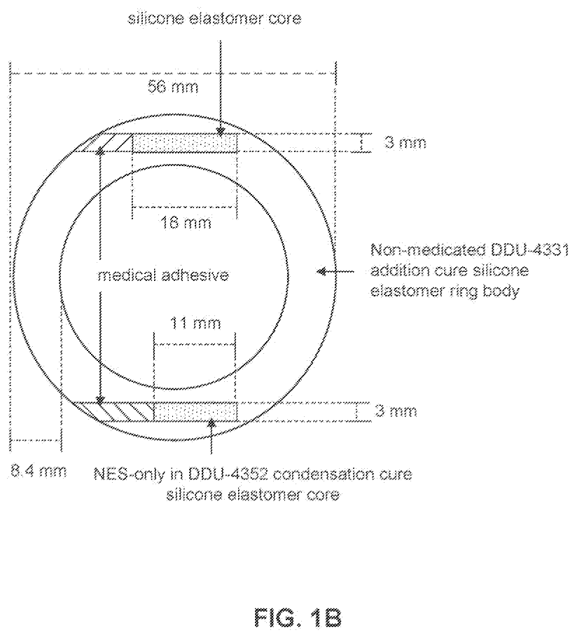

Segesterone acetate has been found to exist in at least two polymorphic non-solvated forms (Polymorphic form I and Polymorphic form II). Polymorphic forms I and II can be obtained by crystallization under conditions known in the art (see modifications A and B, respectively in Hungarian Patent HU0004967). XRPD patterns for each polymorphic form are shown in FIG. 2, which compares a representative core containing both EE and SA to the historical patterns of each of forms I and II.

In some embodiments, the SA used in the vaginal system described herein can be a pure, or substantially pure, single polymorphic form, such as Polymorphic form I or Polymorphic form II. In some embodiments, however, the SA used in the vaginal system described herein can comprises a mixture of polymorphic forms. For example, and in some embodiments, the SA can comprise from approximately 60% to approximately 99% of Polymorphic form I, by weight, with the remainder being the other known polymorphic form, amorphous SA, or a combination thereof. In some embodiments, the SA can comprise from approximately 70% to approximately 99% of Polymorphic form I. In some embodiments, the SA can comprise from approximately 80% to approximately 99% of Polymorphic form I. Each of the percentages specified is percent by weight.

In some embodiments, the SA contained within each core of the vaginal system can comprise from approximately 1% to approximately 40%, by weight, of Polymorphic form II, with the remainder being the other known polymorphic form, amorphous SA, or a combination thereof. In some embodiments, the SA can comprise from approximately 1% to approximately 30% Polymorphic form II. In some embodiments, the SA can comprise from approximately 1% to approximately 20% Polymorphic form II. In some embodiments, the SA can comprise a detectable amount of Polymorphic form II, but less than 10% Polymorphic form II. All percentages noted above are percent by weight.

Applicants have surprisingly discovered that SA particle size is important for obtaining elastomer core mixes, i.e., pre-core mixtures, that are suitable for extrusion and injection molding. If the SA particles are too large, the resulting pre-core mixture is too soft and thus not suitable for extrusion and/or injection molding. Alternatively, if the SA particle size is too small, the resulting pre-core mixture is too stiff for extrusion and/or injection molding. Particle size also influences the rate at which the compound solubilizes into the core and ultimately affects the release profile of the SA from the system into the subject.

In some embodiments, the SA contained within each core of the vaginal system described herein can be micronized. In some embodiments, the SA contained within each core can have a particle size distribution such that at least 95% of the particles have a particle size of from approximately 0.1 microns to approximately 25 microns, from approximately 0.1 microns to approximately 24 microns, from approximately 0.1 microns to approximately 23 microns, from approximately 0.1 microns to approximately 22 microns, from approximately 0.1 microns to approximately 21 microns, or from approximately 0.1 microns to approximately 20 microns.

In some embodiments, the SA contained within each core can have a particle size distribution wherein approximately 90% of the particles have a particle size from approximately 0.5 microns to approximately 15 microns, from approximately 0.5 microns to approximately 14 microns, from approximately 0.5 microns to approximately 13 microns, from approximately 0.5 microns to approximately 12 microns, from approximately 0.5 microns to approximately 11 microns, or from approximately 0.5 microns to approximately 10 microns.

In some embodiments, the SA contained within each core can have a particle size distribution wherein approximately 50% of the particles have a particle size from approximately 0.5 microns to approximately 10 microns, from approximately 0.5 microns to approximately 9 microns, from approximately 0.5 microns to approximately 8 microns, from approximately 0.5 microns to approximately 7 microns, from approximately 0.5 microns to approximately 6 microns, or from approximately 0.5 microns to approximately 5 microns.

In certain embodiments, the SA contained within each core can have a particle size distribution such that not less than 99% of the particles are less than 100 microns. In some embodiments, the SA contained within each core can have a particle size distribution such that not less than 99% of the particles are less than 90 microns. In some embodiments, the SA contained within each core can have a particle size distribution such that not less than 99% of the particles are less than 80 microns. In some embodiments, the SA contained within each core can have a particle size distribution such that not less than 99% of the particles are less than 70 microns. In some embodiments, the SA contained within each core can have a particle size distribution such that not less than 99% of the particles are less than 60 microns. In some embodiments, the SA contained within each core can have a particle size distribution such that not less than 99% of the particles are less than 50 microns. In some embodiments, the SA contained within each core can have a particle size distribution such that not less than 99% of the particles are less than 40 microns. In some embodiments, the SA contained within each core can have a particle size distribution such that not less than 99% of the particles are less than 30 microns. In some embodiments, the SA contained within each core can have a particle size distribution such that not less than 99% of the particles are less than 20 microns. In some embodiments, the SA contained within each core can have a particle size distribution such that not less than 99% of the particles are less than 10 microns.

In certain embodiments, the SA contained within each core can have a D90 less than or equal to approximately 100 microns. In some embodiments, the SA contained within each core can have a D90 less than or equal to approximately 90 microns. In some embodiments, the SA contained within each core can have a D90 less than or equal to approximately 80 microns. In some embodiments, the SA contained within each core can have a D90 less than or equal to approximately 70 microns. In some embodiments, the SA contained within each core can have a D90 less than or equal to approximately 60 microns. In some embodiments, the SA contained within each core can have a D90 less than or equal to approximately 50 microns. In some embodiments, the SA contained within each core can have a D90 less than or equal to approximately 40 microns. In some embodiments, the SA contained within each core can have a D90 less than or equal to approximately 30 microns. In some embodiments, the SA contained within each core can have a D90 less than or equal to approximately 20 microns. In certain embodiments, the SA contained within each core can have a D90 less than or equal to approximately 15 microns. In certain embodiments, the SA contained within each core can have a D90 less than or equal to approximately 12 microns. In some embodiments, the SA contained within each core can have a D90 less than or equal to approximately 10 microns. In certain embodiments, the SA contained within each core can have a D90 less than or equal to approximately 8 microns. In certain embodiments, the SA contained within each core can have a D90 less than or equal to approximately 6 microns.

In certain embodiments, the SA can have a D50 less than or equal to approximately 75 microns. In certain embodiments, the SA can have a D50 less than or equal to approximately 65 microns. In certain embodiments, the SA can have a D50 less than or equal to approximately 55 microns. In certain embodiments, the SA can have a D50 less than or equal to approximately 45 microns. In certain embodiments, the SA can have a D50 less than or equal to approximately 35 microns. In certain embodiments, the SA can have a D50 less than or equal to approximately 25 microns. In certain embodiments, the SA can have a D50 less than or equal to approximately 15 microns. In some embodiments, the SA can have a D50 less than or equal to approximately 10 microns. In some embodiments, the SA can have a D50 less than or equal to approximately 8 microns. In some embodiments, the SA can have a D50 less than or equal to approximately 5 microns. In some embodiments, the SA can have a D50 less than or equal to approximately 3 microns. In some embodiments, the SA can have a D50 less than or equal to approximately 2 microns.

In certain embodiments, the SA can have a D10 greater than or equal to approximately 50 microns. In some embodiments, the SA can have a D10 greater than or equal to approximately 40 microns. In some embodiments, the SA can have a D10 greater than or equal to approximately 30 microns. In some embodiments, the SA can have a D10 greater than or equal to approximately 20 microns. In some embodiments, the SA can have a D10 greater than or equal to approximately 10 microns. In some embodiments, the SA can have a D10 greater than or equal to approximately 5 microns. In some embodiments, the SA can have a D10 greater than or equal to approximately 3 microns. In some embodiments, the SA can have a D10 greater than or equal to approximately 1 micron. In some embodiments, the SA can have a D10 greater than or equal to approximately 0.6 microns. In some embodiments, the SA can have a D10 greater than or equal to approximately 0.5 microns. In some embodiments, the SA can have a D10 greater than or equal to approximately 0.4 microns.

In certain embodiments, the SA contained within each core can have a D90 less than or equal to approximately 80 microns, a D50 less than or equal to approximately 45 microns, and a D10 greater than or equal to approximately 10 microns. In some embodiments, the SA contained within each core can have a D90 less than or equal to approximately 40 microns, a D50 less than or equal to approximately 25 microns, and a D10 greater than or equal to approximately 5 microns. In some embodiments, the SA contained within each core can have a D90 less than or equal to approximately 20 microns, a D50 less than or equal to approximately 15 microns, and a D10 greater than or equal to approximately 1 micron. In some embodiments, the SA contained within each core can have a D90 less than or equal to approximately 10 microns, a D50 less than or equal to approximately 5 microns, and a D10 greater than or equal to approximately 0.6 microns. In some embodiments, the SA contained within each core can have a D90 less than or equal to approximately 8 microns, a D50 less than or equal to approximately 3 microns, and a D10 greater than or equal to approximately 0.4 microns. In some embodiments, the SA contained within each core can have a D90 less than or equal to approximately 6 microns, a D50 less than or equal to approximately 2 microns, and a D10 greater than or equal to approximately 0.2 microns.

In certain embodiments, the vaginal system comprises a second core which comprises SA and EE. In some embodiments, the second core comprises a one or more condensation cure silicone elastomers. In some embodiments, the second core comprises a single condensation cure silicone elastomer. In some embodiments, the condensation cure silicone elastomer is selected from the group consisting of NuSil™ MED-6603 (formerly known as DDU-4352), NuSil™ MED3-6603, NuSil™ MED-6381, NuSil™ MED-6382, and NuSil™ MED-6385, as described elsewhere herein. In certain embodiments, the second core comprises NuSil™ MED-6603 (formerly known as DDU-4352). This material is commercially available.

In certain embodiments, the second core comprises a single elastomer, SA, and EE. In some embodiments, the second core can be prepared by blending the elastomer and the EE. In some embodiments, SA is added to the blend in batches. In some embodiments, the resulting mixture containing the elastomer, EE, and SA can be divided into smaller batches before treatment with a curing agent. In some embodiments, the curing agent can be a tin catalyst. In some embodiments, the curing agent can be dibutyltin dilaurate. In some embodiments, the curing agent is NuSil™ MED-6603 Part B. In some embodiments, the resulting mixture can be extruded into strings after addition of the curing agent.

Applicants have surprisingly discovered that the temperature and relative humidity at which the second core is cured can be important to the rate at which the EE is released on Day 1 of the first product-use cycle. Higher curing temperatures and higher relative humidity during the curing process cause an unacceptable EE burst on Day 1. This effect was not seen in the core containing only SA. In certain embodiments, the strings containing EE and SA can be cured at a temperature below approximately 120° C. In some embodiments, the strings can be cured at a temperature of approximately room temperature to approximately 115° C. In some embodiments, the strings can be cured at a temperature of from approximately 40° C. to approximately 110° C. In some embodiments, the strings can be cured at a temperature of from approximately 50° C. to approximately 100° C. In some embodiments, the strings can be cured at a temperature of from approximately 60° ° C. to approximately 90° C. In some embodiments, the strings can be cured at a temperature of from approximately 90° C. In some embodiments, the strings can be cured at a temperature of approximately 60° C.

In some embodiments, the amount of time that the strings are cured increases with a decrease in curing temperature. In certain embodiments, the strings can be cured for from approximately 5 minutes to approximately 60 minutes. In some embodiments, the strings can be cured for from approximately 25 minutes to approximately 50 minutes. In some embodiments, the strings can be cured for from approximately 30 minutes to approximately 45 minutes. In some embodiments, the strings can be cured for from approximately 30 minutes. In some embodiments, the strings can be cured at approximately 90° C. for approximately 10 minutes. In some embodiments, the strings can be cured at approximately 60° C. for approximately 15 to approximately 20 minutes.

In certain embodiments, the strings can be cured at a relative humidity of less than approximately 5%. In certain embodiments, the strings can be cured at a relative humidity of less than approximately 4%. In some embodiments, the strings can be cured at a relative humidity of less than approximately 3%. In some embodiments, the strings can be cured at a relative humidity of less than approximately 2%. In some embodiments, the strings can be cured at a relative humidity from approximately 1% to approximately 2%. In some embodiments, the strings can be cured at a relative humidity of approximately 1.8%.

In some embodiments, the cured product can be post-cured at room temperature for at least 2 days. In some embodiments, the cured product can be post-cured at room temperature for at least 3 days. In some embodiments, the cured product can be post-cured at room temperature for at least 4 days. In some embodiments, the cured product can be post-cured at room temperature for at least 5 days. In some embodiments, the cured product can be post-cured at room temperature for at least 6 days. In some embodiments, the cured product can post-cured at room temperature for at least 7 days. In some embodiments, the cured product can be post-cured at room temperature for at least 8 days. In some embodiments, the cured product can be post-cured at room temperature for at least 9 days. In some embodiments, the cured product can be post-cured at room temperature for at least 10 days.

In some embodiments, the strings can be cut after the post-cure period to provide the cores. In some embodiments, the strings can be cut to a length from approximately 15 mm to approximately 21 mm. In some embodiments, the strings can be cut to a length from approximately 16 mm to approximately 20 mm. In some embodiments, the strings can be cut to a length from approximately 17 mm to approximately 19 mm. In some embodiments, strings are cut to a length of approximately 18 mm. In certain embodiments, the weight of the second core can be from approximately 115 mg to approximately 175 mg. In certain embodiments, the weight of the second core can be from approximately 125 mg to approximately 165 mg. In some embodiments, the weight of the second core can be from approximately 135 mg to approximately 155 mg. In some embodiments, the weight of the second core can be approximately 145 mg.

In certain embodiments, the second core can contain from approximately 40 mg to approximately 80 mg of SA. In certain embodiments, the second core can contain from approximately 50 mg to approximately 70 mg of SA. In some embodiments, the second core can contain from approximately 50 mg to approximately 60 mg of SA. In some embodiments, the second core can contain from approximately 55 mg to approximately 60 mg of SA. In some embodiments, the second core can contain approximately 58 mg of SA, or from 56 to 60 mg SA.

In some embodiments, the second core can contain from approximately 14 mg to approximately 25 mg of EE. In some embodiments, the second core can contain from approximately 15 mg to approximately 20 mg of EE. In some embodiments, the second core can contain from approximately 16 mg to approximately 19 mg of EE. In some embodiments, the second core can contain from approximately 15 mg to approximately 18 mg of EE. In some embodiments, the second core can contain from approximately 16 mg to approximately 18 mg of EE. In some embodiments, the second core can contain approximately 17.4 mg of EE, or from 17.2 to 17.6 mg of EE.

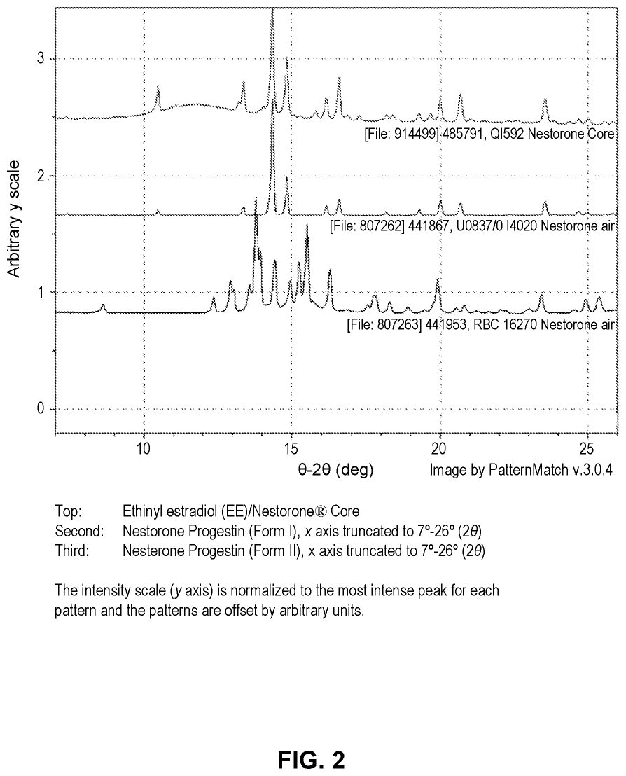

Crystalline forms of EE and multiple crystalline EE hydrates are known in the literature (see, for example, Pheasant, R., “Polymorphism of 17-Ethinylestradiol”, J. Am. Chem. Soc. 1950, 72 (9), pp 4303-4304 and Guguta, C. et al., Cryst. Growth Des. 2008, 8 (3), pp 823-831 which are both incorporated by reference in their entireties). A comparison of the XRPD pattern of the EE API to the calculated XRPD patterns of EE hemihydrate and anhydrous EE are shown in FIG. 3. In some embodiments, the EE contained within the second core comprises one or more anhydrous forms. In some embodiments, the EE contained within the second core comprises one or more hemihydrate forms. In some embodiments, the EE contained within the second core comprises a mixture of one or more anhydrous forms and one or more hemihydrate forms. In certain embodiments, the EE contained within the second core comprises a crystalline form that melts from approximately 181° C. to approximately 186° C. In some embodiments, the EE comprises a crystalline form that melts from approximately 141 to approximately 146° C. In yet another embodiment, the EE contained within the second core comprises a mixture of a crystalline form that melts from approximately 181° ° C. to approximately 186° C. and a crystalline form that melts from approximately 141 to approximately 146° C., wherein the ratio of these crystalline forms ranges from approximately 99:1 to approximately 1:99, by weight.

As discussed herein, particle size influences the rate at which the compound solubilizes into the core and ultimately affects the release profile of the EE from the system into the subject. In some embodiments, the EE contained within the second core of the vaginal system can be micronized. In some embodiments, the EE contained within the second core can have maximum particle size from approximately 10 microns to approximately 20 microns. In some embodiments, the EE contained within the second core can have maximum particle size from approximately 11 microns to approximately 19 microns. In some embodiments, the EE contained within the second core can have maximum particle size from approximately 12 microns to approximately 18 microns. In some embodiments, the EE contained within the second core can have maximum particle size from approximately 13 microns to approximately 17 microns. In some embodiments, the EE contained within the second core can have maximum particle size from approximately 14 microns to approximately 16 microns. In some embodiments, the EE can have a maximum particle size of approximately 15 microns.

In some embodiments, the EE can have a particle size distribution wherein approximately 99% of the particles have a maximum particle size from approximately 11 microns to approximately 15 microns. In some embodiments, the EE can have a particle size distribution wherein approximately 99% of the particles have a maximum particle size from approximately 12 microns to approximately 14 microns. In some embodiments, the EE can have a particle size distribution wherein approximately 99% of the particles have a maximum particle size from approximately 12 microns to approximately 13 microns. In some embodiments, the EE can have a particle size distribution wherein approximately 99% of the particles have a maximum particle size of approximately 12.5 microns. In some embodiments, the EE can have a particle size distribution wherein approximately 95% of the particles have a maximum particle size from approximately 8 microns to approximately 13 microns. In some embodiments, the EE can have a particle size distribution wherein approximately 95% of the particles have a maximum particle size from approximately 9 microns to approximately 12 microns. In some embodiments, the EE can have a particle size distribution wherein approximately 95% of the particles have a maximum particle size from approximately 9 microns to approximately 11 microns. In some embodiments, the EE can have a particle size distribution wherein approximately 95% of the particles have a maximum particle size of approximately 10.0 microns. In some embodiments, the EE can have a particle size distribution wherein approximately 50% of the particles have a maximum particle size from approximately 1 micron to approximately 4 microns. In some embodiments, the EE can have a particle size distribution wherein approximately 50% of the particles have a maximum particle size from approximately 2 microns to approximately 4 microns. In some embodiments, the EE can have a particle size distribution wherein approximately 50% of the particles have a maximum particle size of approximately 3 microns. In some embodiments, the EE can have a particle size distribution wherein approximately 40% or less of the particles have a particle size less than or equal to approximately 2 microns. In some embodiments, the EE can have a particle size distribution wherein approximately 40% or less of the particles have a particle size less than or equal to approximately 1.5 microns. In some embodiments, the EE can have a particle size distribution wherein approximately 40% or less of the particles have a particle size less than or equal to approximately 1.3 microns.

It has been surprisingly discovered that the age of the second core upon assembly into the ring body impacts the initial burst of EE on Day 1. For example, newer cores were shown to provide an unacceptable EE burst on Day 1. In certain embodiments, post curing, one or more of the cores can be stored for at least 8 days before assembling into the ring body. In certain embodiments, post-curing, one or more of the cores can be stored for at least 10 days before assembling into the ring body. In certain embodiments, post-curing, one or more of the cores can be stored for at least 12 days before assembling into the ring body. In certain embodiments, post-curing, one or more of the cores can be stored for at least 14 days before assembling into the ring body. In certain embodiments, post-curing, one or more of the cores can be stored for at least 16 days before assembling into the ring body. In certain embodiments, post-curing, one or more of the cores can be stored for at least 18 days before assembling into the ring body. In certain embodiments, post-curing, one or more of the cores can be stored for at least 20 days before assembling into the ring body. In certain embodiments, post-curing, one or more of the cores can be stored for at least 21 days before assembling into the ring body. In certain embodiments, post-curing, one or more of the cores can be stored for at least 22 days before assembling into the ring body. In certain embodiments, post-curing, one or more of the cores can be stored for at least 23 days before assembling into the ring body. In certain embodiments, post-curing, one or more of the cores can be stored for at least 24 days before assembling into the ring body. In certain embodiments, post-curing, one or more of the cores can be stored for at least 25 days before assembling into the ring body. In certain embodiments, post-curing, one or more of the cores can be stored for at least 26 days before assembling into the ring body. In certain embodiments, post-curing, one or more of the cores can be stored for at least 27 days before assembling into the ring body. In certain embodiments, post-curing, one or more of the cores can be stored for at least 28 days before assembling into the ring body. In certain embodiments, post-curing, one or more of the cores can be stored for at least 29 days before assembling into the ring body. In certain embodiments, post-curing, one or more of the cores can be stored for at least 30 days before assembling into the ring body. In certain embodiments, post-curing, one or more of the cores can be stored for at least 31 days before assembling into the ring body. In certain embodiments, post-curing, one or more of the cores can be stored for at least 32 days before assembling into the ring body. In certain embodiments, post-curing, one or more of the cores can be stored for at least 33 days before assembling into the ring body. In certain embodiments, post-curing, one or more of the cores can be stored for at least 34 days before assembling into the ring body. In certain embodiments, post-curing, one or more of the cores can be stored for at least 35 days before assembling into the ring body.

In certain embodiments, the shaping of the pre-core mixture, the cutting of the resulting cores, and/or storage of the resulting cores can be performed at a temperature of from approximately 10° ° C. to approximately 40° C. In certain embodiments, the shaping of the pre-core mixture, the cutting of the resulting cores, and/or storage of the resulting cores can be performed at a temperature of from approximately 15° C. to approximately 35° C. In certain embodiments, the shaping of the pre-core mixture, the cutting of the resulting cores, and/or storage of the resulting cores can be performed at a temperature of from approximately 15° C. to approximately 30° C. In some embodiments, the shaping of the pre-core mixture, the cutting of the resulting cores, and/or storage of the resulting cores can be performed at a temperature of from approximately 20° C. to approximately 25° C.

In some embodiments, the shaping of the pre-core mixture, the cutting of the resulting cores, and/or storage of the resulting cores can be performed at a relative humidity of greater than or equal to approximately 10%. In some embodiments, the shaping of the pre-core mixture, the cutting of the resulting cores, and/or storage of the resulting cores can be performed at a relative humidity of greater than or equal to approximately 20%. In some embodiments, the shaping of the pre-core mixture, the cutting of the resulting cores, and/or storage of the resulting cores can be performed at a relative humidity of greater than or equal to approximately 30%. In some embodiments, the shaping of the pre-core mixture, the cutting of the resulting cores, and/or storage of the resulting cores can be performed at a relative humidity of greater than or equal to approximately 40%.

In some embodiments, the cores of the vaginal system described herein conform to the guidelines outlined in the US Pharmacopeial Convention, incorporated herein by reference, and in particular USP <905>.

### Ring Body

The vaginal system ring body typically comprises one or more polymers. In certain embodiments, the ring body comprises one or more polymers selected from a polystyrene, a thermoplastic polymer (including, but not limited to, poly(methyl methacrylate), acrylonitrile butadiene styrene, nylon, polylactic acid, polybenimidazole, polycarbonate, polyether sulfone, polyoxymethylene, polyetherketone, polyetherimide, polyethylene, polyphenylene oxide, polyphenylene sulfide, polypropylene, polystyrene, polyvinyl chloride, polyvinylidene floride, and teflon), and elastomers (including, but not limited to natural and synthetic polyisoprene, polybutadiene, chloroprene, butyl rubber (including halogenated derivatives thereof), styrene-butadiene, nitrile rubber (including halogenated derivatives thereof), ethylene/propylene rubbers (including both melt blends and reactor blends (block copolymers) of ethylene and propyelene), epichlorohydrin rubber, polyacrylic rubber, a silicone elastomer, fluorosilicone rubber, a fluoroelastomer (e.g. VITON, TECNOFLON, FLUOREL, AFAS, and DAI-EL), a perfluoroelastomer, a polyether block amide, chlorosulfonated polyethylene, ethylene vinylacetate (“EVA”)). In some embodiments, the ring body comprises EVA. In some embodiments, the ring body comprises one or more elastomers wherein the elastomers are silicone elastomers. In some embodiments, the ring body comprises a mixture of silicone and other elastomers. In some embodiments, the ring body comprises a single silicone elastomer. In other embodiments, the ring body comprises multiple silicone elastomers. In some embodiments the ring body comprises a condensation-cure silicone elastomer. In other embodiments, the ring body comprises an addition-cure silicone elastomer.

In some embodiments, the ring body comprises a silicone addition-cure elastomer. Addition-cure systems silicone elastomers typically include vinyl-terminated silicone polymers, a platinum catalyst, and a silyl-hydride cross-linker. In general, silicone addition-cure elastomers are supplied as two-part systems that need to be intimately mixed to initiate curing. That said, and in other embodiments, the addition cure silicone elastomers can be supplied pre-mixed as non-polymerized starting materials, with a separate catalyst, or in three distinct component parts which are subsequently mixed in an appropriate ratio.

In certain embodiments, the ring body comprises a medical grade addition-cure silicone elastomer having a platinum concentration from approximately 1 ppm to approximately 15 ppm. In certain embodiments, the ring body comprises a medical grade addition-cure silicone elastomer having a platinum concentration from approximately 2 ppm to approximately 12 ppm.

In certain embodiments, the ring body comprises a medical grade addition-cure silicone elastomer having a platinum concentration from approximately 2 ppm to approximately 10 ppm. In some embodiments, the addition-cure silicone elastomer can be a polysiloxane elastomer comprising approximately 2 ppm to approximately 10 ppm platinum. In some embodiments, the polysiloxane elastomer can be a diorganopolysiloxane elastomer comprising approximately 2 ppm to approximately 10 ppm platinum. In some embodiments, the diorganopolysiloxane elastomer can be a dimethylpolysiloxane elastomer comprising approximately 2 ppm to approximately 10 ppm platinum. As will be discussed in more detail below, it has been surprisingly discovered that the concentration of platinum in the ring body is believed to play a role in controlling the release rate of EE in the vaginal system. Concentrations of platinum above or below the specified ranges can lead to increased rates of EE sequestration, while insufficient platinum can lead to release of too much EE, and the concomitant side effects associated with too much estradiol.

In addition to having the specified platinum concentration, the addition-cure silicone elastomer can also comprise one or more opacity agents, one or more pigments, one or more antidegradants, one or more fillers, or combinations thereof.

In certain embodiments, the addition-cure silicone elastomer having the specified platinum concentration can be prepared from two components, “Part A” and “Part B.” In some embodiments, the first part (Part A), contains uncured vinyl-terminated silicone polymers and a platinum catalyst which acts as a curing agent. In some embodiments, the second part (Part B) contains uncured vinyl-terminated silicone polymers and a hydride cross-linker. In certain embodiments, the ratio of hydride cross-linker (“hydride”) to vinyl-terminated polymer (“vinyl”) within both Part A and Part B is from approximately 1:2 to approximately 5:1. In some embodiments, the hydride/vinyl ratio is from approximately 1:1.5 to approximately 4:1. In some embodiments, the hydride/vinyl ratio is from approximately 1:1.5 to approximately 3:1. In some embodiments, the hydride/vinyl ratio is from approximately 1:1.5 to approximately 2:1. In some embodiments, the hydride/vinyl ratio is from approximately 1:1.5 to approximately 1.5:1. In some embodiments, the hydride/vinyl ratio is from approximately 1:1 to approximately 1.3:1. In some embodiments, the hydride/vinyl ratio is from approximately 1:1 to approximately 1.2:1.

Increasing the ratio of Part A to Part B has been found to increase both tensile strength and elongation of the cured elastomer without affecting the Shore A hardness. Accordingly, the appropriate ratios of Part A and Part B can be selected to provide an elastomer that had enough flexibility as to be easy to insert and remove yet be durable enough to withstand the physical stress of use. In certain embodiments, the ring body elastomer can be prepared by mixing an approximately 8:1 to approximately 12:1 ratio of Part A to Part B. In certain embodiments, the addition-cure silicone elastomer can be prepared by mixing an approximately 9:1 to approximately 11:1 ratio of Part A to Part B. In certain embodiments, the addition-cure silicone elastomer can be prepared by mixing an approximately 9.5:1 to approximately 10.5:1 ratio of Part A to Part B. In certain embodiments, the addition-cure silicone elastomer can be prepared by mixing an approximately 10:1 ratio of Part A to Part B.

In some embodiments, the addition-cure silicone elastomer having the noted platinum concentration can be NuSil™ MED-4870. NuSil™ MED-4870 can be prepared by mixing two components, “Part A” and “Part B.” In addition to siloxanes and silicones, Part A in this embodiment can comprise 30-40% trimethylsilylsilanamine (CAS No. 68909-20-6). Part B in this embodiment can comprise dimethylsiloxanes and dimethylsilicones, as well as 30-40% trimethylsilylsilanamine (CAS No. 689-20-6) and a platinum catalyst.

In some embodiments, the addition-cure silicone elastomer having a platinum concentration within the specified ranges can be NuSil™ DDU-4320. Like other addition-cure silicone elastomers, NuSil™ DDU-4320 can be prepared by mixing appropriate ratios of two components, “Part A” and “Part B.” In this embodiment, Part A can comprise 40-50% vinyl-terminated dimethylsiloxanes and dimethylsilicones (CAS No. 68952-0001), 10-20% amorphous, fumed, crystalline-free silica (CAS No. 112945-52-5), and <1% hydroxyl-1% hydroxyl-terminated dimethyl and methyl-vinylsiloxanes and silicones (CAS No. 67923-19-7). In some embodiments, Part B comprises 40-50% vinyl-terminated dimethylsiloxanes and dimethylsilicones (CAS No. 68952-0001), 30-40% ethenyldimethylsilyloxy- and trimethylsilyloxy-modified silica (CAS No. 68988-89-6), 10-20% amorphous, fumed, crystalline-free silica (CAS No. 112945-52-5), <1% silicic acid tetraethyl ester (CAS No. 68988-57-8), <1% 1-ethynylcyclohexanol (CAS No. 78-27-3), and <1% hydroxyl-terminated dimethyl and methyl-vinylsiloxanes and silicones (CAS No. 67923-19-7).

In some embodiments, the addition-cure silicone elastomer having a platinum concentration within the specified ranges can be MED4-4224 (previously known as DDU-4331). As above, this addition-cure silicone elastomer can be prepared by mixing appropriate ratios of two components, “Part A” and “Part B.” In this embodiment, Part A comprises 65-75% mono(vinyl group) terminated dimethylsiloxanes and dimethylsilicones (CAS No. 68952-00-1), 15-20% amorphous, fumed, crystalline-free silica (CAS No. 112945-52-5), and <5% titanium dioxide (CAS No. 137463-67-7). Part B, in this embodiment, comprises 65-75% mono(vinyl group) terminated dimethylsiloxanes and dimethylsilicones (CAS No. 68952-00-1), 10-15% siloxanes and silicones (dimethyl and methyl) (CAS No. 68037-59-2), and a platinum catalyst.

In some embodiments, the silicone elastomer is NuSil™ MED4-4224 (previously known as DDU-4331). In some embodiments, in addition to the components noted above, the NuSil™ MED4-4224 comprises one or more opacity agents. In some embodiments, the opacity agent is titanium dioxide. In some embodiments, the NuSil™ MED4-4224 comprises approximately 4% TiO₂ by weight.

In some embodiments, the ring body can be formed when the component parts of the addition-cure silicone elastomer are mixed and then molded into ring bodies and subjected to curing conditions. In some embodiments, the ring bodies can be cured at a temperature of from approximately 120° C. to approximately 180° C. In some embodiments, the ring bodies can be cured at a temperature of from approximately 130° C. to approximately 170° C. In some embodiments, the ring bodies can be cured at a temperature of from approximately 140° C. to approximately 160° C. In some embodiments, the ring bodies are cured at a temperature of from approximately 145° C. to approximately 155° C. In some embodiments, the ring bodies can be cured from approximately 20 to approximately 210 seconds. In some embodiments, the ring bodies can be cured from approximately 30 to approximately 200 seconds. In some embodiments, the ring bodies can be cured from approximately 40 to approximately 190 seconds. In some embodiments, the ring bodies can be cured from approximately 50 to approximately 190 seconds. In some embodiments, the ring bodies can be cured from approximately 60 to approximately 180 seconds. In some embodiments, the ring bodies can be cured for approximately 180 seconds.

In certain embodiments, the cured elastomer ring body has a specific gravity of from approximately 1 to approximately 1.5. In some embodiments, the cured elastomer ring body has a specific gravity of from approximately 1.05 to approximately 1.4. In some embodiments, the cured elastomer ring body has a specific gravity of from approximately 1.05 to approximately 1.3. In some embodiments, the cured elastomer ring body has a specific gravity of from approximately 1.05 to approximately 1.25. In some embodiments, the cured elastomer ring body has a specific gravity of from approximately 1.05 to approximately 1.20. In some embodiments, the cured elastomer ring body has a specific gravity of from approximately 1.07 to approximately 1.17. In some embodiments, the cured elastomer ring body has a specific gravity of from approximately 1.08 to approximately 1.11.

In certain embodiments, the ring bodies can be removed from the mold and allowed to rest before inserting the cores. In some embodiments, the ring bodies can be rested at a temperature of from approximately 10° C. to approximately 40° C. In some embodiments, the ring bodies can be rested at a temperature of from approximately 15° C. to approximately 35° C. In some embodiments, the ring bodies can be rested at a temperature of from approximately 15° C. to approximately 30° C. In some embodiments, the ring bodies can be rested at a temperature from approximately 19° C. to approximately 25° C. In some embodiments, the ring bodies can be rested for a period of from approximately 10 to approximately 45 days. In some embodiments, the ring bodies can be rested for a period of from approximately 20 to approximately 40 days. In some embodiments, the ring bodies are rested for approximately 30 days.

As noted elsewhere herein, the ring body contains one or more channels adapted to receive the active-impregnated cores. In certain embodiments, the channels adapted to receive the cores can be created within the ring bodies during the molding process. Alternatively, any suitable means for creating the channel after the molding process is complete can also be used. For example, and in some embodiments, the channels can be prepared by laser or by using an appropriate cutting mechanism, such as a metal blade or high-pressure water. In some embodiments, the channels can be created by puncturing. In some embodiments, the channels can be created by drilling. An appropriate mechanism for introducing the one or more channels into the ring body can be selected depending upon channel placement and size and other factors. As noted elsewhere herein, the channel or channels adapted to receive the core(s) can be a bore, such as a cylindrical bore adapted to receive an appropriately shaped cylindrical or spherical core. In other embodiments, the channel or channels can be adapted to receive a core or cores shaped like a rectangular prism, including for example a square prism, or a core or cores shaped like a cone, a triangular prism, a triangular pyramid, a rectangular pyramid, a pentagonal prism, a hexagonal prism, a heptagonal prism, or any other three-dimensional shape suitable for manufacture. In some embodiments, the channel or channels can be adapted to receive a core or cores that are disc shaped. In certain embodiments, the channel or channels can be adapted to receive a cylindrical core or core shaped like a rectangular prism.

Curing results in hardening of the resulting ring body. In certain embodiments, the cured ring body has a mean elongation parallel to the cores of between approximately 350 and approximately 550%. In some embodiments, the cured ring body has a mean elongation parallel to the cores of between approximately 375 and approximately 525%. In some embodiments, the cured ring body has a mean elongation parallel to the cores of between approximately 400 and approximately 500%. In some embodiments, the cured ring body has a mean elongation parallel to the cores of approximately 418%. In certain embodiments, the cured ring body has a mean elongation perpendicular to the cores of between approximately 350 and approximately 550%. In some embodiments, the cured ring body has a mean elongation perpendicular to the cores of between approximately 375 and approximately 525%. In some embodiments, the cured ring body has a mean elongation perpendicular to the cores of between approximately 400 and approximately 500%. In some embodiments, the cured ring body has a mean elongation perpendicular to the cores of approximately 474%.

In certain embodiments, the cured ring body has a mean tensile strength parallel to the cores of from approximately 9,000 N/mm² to approximately 10,000 N/mm². In some embodiments, the cured ring body has a mean tensile strength parallel to the cores of from approximately 9,100 N/mm² to approximately 9,750 N/mm². In some embodiments, the cured ring body has a mean tensile strength parallel to the cores of from approximately 9,200 N/mm² to approximately 9,500 N/mm². In some embodiments, the cured ring body has a mean tensile strength parallel to the cores of from approximately 9,300 N/mm² to approximately 9,400 N/mm². In some embodiments, the cured ring body has a mean tensile strength parallel to the cores of approximately 9312 N/mm². In certain embodiments, the cured ring body has a mean tensile strength perpendicular to the cores of from approximately 10,000 N/mm² to approximately 11,000 N/mm². In some embodiments, the cured ring body has a mean tensile strength perpendicular to the cores of from approximately 10,100 N/mm² to approximately 10,750 N/mm². In some embodiments, the cured ring body has a mean tensile strength perpendicular to the cores of from approximately 10,200 N/mm² to approximately 10,500 N/mm². In some embodiments, the cured ring body has a mean tensile strength perpendicular to the cores of from approximately 10,300 N/mm² to approximately 10,400 N/mm². In some embodiments, the cured ring body has a mean tensile strength perpendicular to the cores of approximately 10,369 N/mm².

In certain embodiments, the cured ring body has a mean fatigue parallel to the cores between approximately 80 and approximately 110%. In some embodiments, the cured ring body has a mean fatigue parallel to the cores between approximately 85 and approximately 105%. In some embodiments, the cured ring body has a mean fatigue parallel to the cores between approximately 90 and approximately 100%. In some embodiments, the cured ring body has a mean fatigue parallel to the cores of approximately 95%. In certain embodiments, the cured ring body has a mean fatigue perpendicular to the cores between approximately 80 and approximately 100%. In some embodiments, the cured ring body has a mean fatigue perpendicular to the cores between approximately 85 and approximately 100%. In some embodiments, the cured ring body has a mean fatigue perpendicular to the cores between approximately 90 and approximately 100%. In some embodiments, the cured ring body has a mean fatigue perpendicular to the cores of approximately 98%.

In some embodiments, the cured elastomer has a shore A hardness of from approximately 10 to approximately 50. In some embodiments, the cured elastomer has a shore A hardness of from approximately 15 to approximately 45. In some embodiments, the cured elastomer has a shore A hardness of from approximately 20 to approximately 40. In some embodiments, the cured elastomer has a shore A hardness of from approximately 25 to approximately 35. In some embodiments, the cured elastomer has a shore A hardness of from approximately 25 to approximately 30.

### Assembly of the Vaginal System

Depending on the configuration, the vaginal system can be completed by inserting an appropriate number of appropriately aged cores into channels or other structures within the ring body adapted to receive the core(s). In some embodiments, one or more suitable medical adhesives can be added to secure the cores in the ring body. In some embodiments, the medical adhesive can be added before the cores are added. In some embodiments, the medical adhesive can be added after the cores are added and in certain embodiments, the medical adhesive can be added before and after the cores are added. In certain embodiments, the medical adhesive can be a one-part acetoxy (alkyltriacetoxysilane) or alcohol (alkoxy) cross-linked cure system. These one-part adhesives cure in the presence of ambient humidity. In some embodiments, the acetoxy cure system utilizes a tin catalyst, while in other embodiments, the acetoxy cure system does not utilize a tin catalyst. In other embodiments, the medical adhesive can be a UV-cure (solvent-free) adhesive. Such adhesives are known in the art and comprise a photoinitiatior that initiates cross linking upon exposure to UV radiation between 200 to 500 nm.

Medical adhesives can be purchased from vendors such as NuSil and Elkem. In some embodiments, the medical adhesive used can be NuSil™ MED-1134, which comprises 15-25% trimethylsilanamine (CAS No. 68909-20-6) and <5% methylsilanetriol triacetate (CAS No. 4253-34-3). In some embodiments, the channels can be sealed with additional medical adhesive. In certain embodiments, and in a ring body containing two channels, the ring can be assembled by adding medical adhesive to each channel, inserting one core, generally an aged core, into each channel, and adding additional medical adhesive to the channels once the cores are added.

In some embodiments, the ring can be assembled at a temperature of from approximately 10° C. to approximately 35° C. In some embodiments, the ring assembly can be conducted at a temperature of from approximately 15° C. to approximately 30° C. In certain embodiments, the ring assembly can be conducted at a relative humidity of from approximately 40% to approximately 95%. In certain embodiments, the ring assembly can be conducted at a relative humidity of from approximately 45% to approximately 90%. In some embodiments, the ring assembly can be conducted at a relative humidity of from approximately 50% to approximately 80%. In some embodiments, the ring assembly can be conducted at a relative humidity of from approximately 50% to approximately 75%. In some embodiments, the ring assembly can be conducted at a relative humidity of from approximately 50% to approximately 65%. In some embodiments, the ring assembly can be conducted at a relative humidity of approximately 55%.

In some embodiments, the vaginal system can be assembled by extruding the ring body about one or more cores.

In some embodiments, the assembled vaginal system can be cured at room temperature for a period of approximately 1 to approximately 14 days. In some embodiments, the assembled vaginal system can be cured at room temperature for a period of approximately 2 to approximately 10 days. In some embodiments, the assembled vaginal system can be cured for a period of approximately 3 to approximately 7 days.

In certain embodiments, the assembled vaginal system has a total weight of approximately 6 grams to approximately 15 grams. In some embodiments, the assembled vaginal system has a total weight of approximately 6 grams to approximately 10 grams. In some embodiments, the assembled vaginal system has a total weight of approximately 8 grams to approximately 10 grams. In some embodiments, the assembled vaginal system has a total weight of approximately 9 grams.

In certain embodiments, the assembled vaginal system can be packaged into a pouch. In some embodiments, the pouch comprises aluminum. In some embodiments, the ring can be packaged at a temperature of from approximately 10° C. to approximately 35° C. In some embodiments, the packaging can be conducted at a temperature of from approximately 15° ° C. to approximately 30° C. In some embodiments, the packaging can be conducted at a relative humidity greater than or equal to 40%. In some embodiments, the packaging can be conducted at a relative humidity of from approximately 40% to approximately 90%. In some embodiments, the packaging can be conducted at a relative humidity of from approximately 50% to approximately 80%. In some embodiments, the packaging can be conducted at a relative humidity of from approximately 50% to approximately 70%. In some embodiments, the packaging can be conducted at a relative humidity of approximately 55%.

In other embodiments, the packaged vaginal system can be matured at a temperature of from approximately 10° C. to approximately 35° C. In certain embodiments, the packaged vaginal system can be matured at a temperature of from approximately 15° C. to approximately 30° C. In certain embodiments, the maturation time can be from approximately 15 to approximately 60 days. In certain embodiments, the maturation time can be from approximately 25 to approximately 40 days. In some embodiments, the maturation time can be from approximately 28 to approximately 35 days.

In some embodiments, the vaginal system described herein operates when the EE and SA partially solubilize into the cores into which they are contained, then diffuse from the cores into the ring body and eventually out of the ring body and into the subject. The system is complex, as the rate of solubilization must be controlled to deliver the proper amount of each agent for each of the four quarterly product-use cycles or for a 365-day product-use cycle. If the agents are too soluble in either the cores or the ring body, too much agent is released, and if too little EE or SA are solubilized into the core or the ring body, an insufficient amount will be released. Stability of the rings over extended periods of time is also essential. That is, the polymeric systems chosen must be compatible with both SA and EE such that sufficient amounts of both SA and EE remain available in sufficient quantities to provide the desired release rate of both active agents over four quarterly product-use cycles or over a 365-day product-use cycle, especially as the vaginal system is repeatedly exposed to twenty one-day periods of heat and humidity once placed in the vagina.

Surprisingly, it was discovered that while the amount of SA recoverable from the vaginal system over 24 months of storage at 25° C. and 60% relative humidity remained essentially constant, the amount of EE recoverable from the system decreased in a time-dependent manner. This was quite surprising as a similar trend was not observed during long-term stability studies on the cores before assembly. In fact, the full amount of EE was found to be recoverable from the core by extraction even after extended storage.

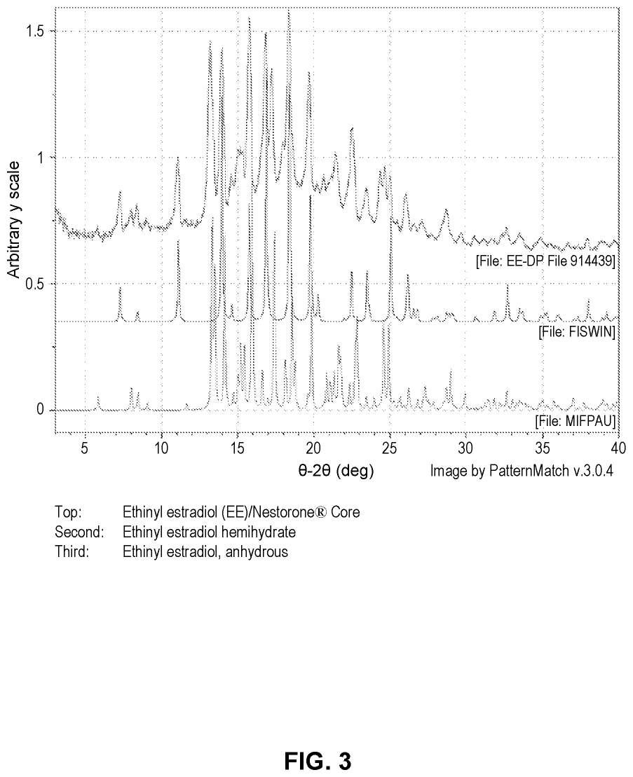

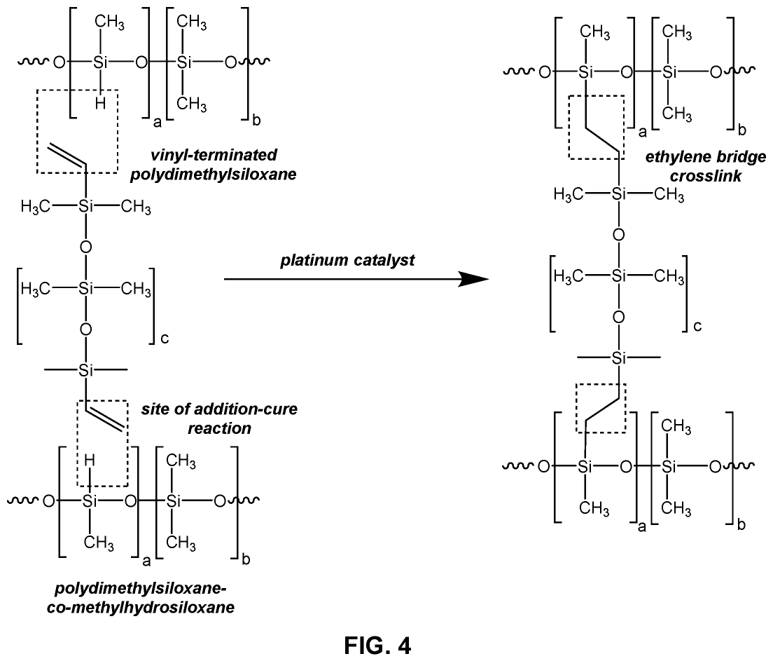

Without being bound to a particular theory, it is believed that platinum dispersed throughout the ring body catalyzes a reaction between excess/unreacted hydrosilane present in the cured ring body elastomer and the terminal acetylene group in the EE as it diffuses into the ring body during maturation of the system. As this process binds the EE to the ring body elastomer, it is not available for release from the ring, causing a decrease in the recoverable amount of EE over time. This process is shown schematically in FIGS. 4 and 5. FIG. 4, for example, shows the process by which an exemplary addition-cure silicone elastomer used to prepare the ring body forms under catalytic conditions. Although this process is generally complete under the conditions described herein, the silicone elastomer resulting from the platinum catalyzed reaction results in an elastomer having platinum catalyst dispersed throughout, along with an amount of unreacted hydrosilanes present on the polymeric backbone. Without wishing to be bound by theory, it is believed that these hydrosilanes are dispersed randomly throughout the ring body, along with the platinum catalyst, which is more evenly dispersed as it is not believed to be linked to the polymer itself. EE, some of which is dissolved in the core, and some of which dissolves into the core over the life of the vaginal system, migrates from the core through the ring body. Most of the EE migrates successfully out of the ring body into a subject's vagina and provides EE over the course of multiple product-use cycles. But a certain number of EE molecules interact with both the platinum catalyst dispersed throughout the ring body and a hydrosilane, resulting in the structure shown in FIG. 5.

To determine if the amount of residual hydride in ring body elastomer contributed to this phenomenon, the effect of the hydride/vinyl ratio of the uncured elastomer on the in vitro release of EE on Day 1 at 6 months and at 12 months was studied. The results showed that higher hydride/vinyl ratios (>1:1) provided lower Day 1 EE releases than lower (<1:1) hydride/vinyl ratios. Surprisingly, Applicants discovered that hydride/vinyl ratios <1 led to EE “bursts” which provided unacceptably high Day 1 releases at 6 months and 12 months. Alternatively, hydride/vinyl ratios from approximately 1:1 to approximately 1.3:1 provided acceptable EE release profiles over the same period of time.

In some embodiments, a hydride/vinyl ratio of <1:1 provides a Day 1 release after six months of storage at 25° C. and 60% relative humidity from approximately 25% to approximately 85% higher than the Day 1 release prior to storage. In some embodiments, a hydride/vinyl ratio of <1:1 provides a Day 1 release after six months of storage at 25° C. and 60% relative humidity from approximately 25% to approximately 80% higher than the Day 1 release prior to storage. In some embodiments, a hydride/vinyl ratio of <1:1 provides a Day 1 release after six months of storage at 25° C. and 60% relative humidity from approximately 30% to approximately 75% higher than the Day 1 release prior to storage. In some embodiments, a hydride/vinyl ratio of <1:1 provides a Day 1 release after six months of storage at 25° C. and 60% relative humidity from approximately 35% to approximately 65% higher than the Day 1 release prior to storage. In some embodiments, a hydride/vinyl ratio of <1:1 provides a Day 1 release after six months of storage at 25° C. and 60% relative humidity from approximately 35% to approximately 60% higher than the Day 1 release prior to storage. In some embodiments, a hydride/vinyl ratio of <1:1 provides a Day 1 release after six months of storage at 25° C. and 60% relative humidity from approximately 35% to approximately 55% higher than the Day 1 release prior to storage. In some embodiments, a hydride/vinyl ratio of <1:1 provides a Day 1 release after six months of storage at 25° C. and 60% relative humidity from approximately 35% to approximately 50% higher than the Day 1 release prior to storage. In some embodiments, a hydride/vinyl ratio of <1:1 provides a Day 1 release after six months of storage at 25° C. and 60% relative humidity from approximately 40% to approximately 45% higher than the Day 1 release prior to storage.

In some embodiments, a hydride/vinyl ratio of >1:1 provides a Day 1 release after six months of storage at 25° C. and 60% relative humidity from approximately 15% lower to approximately 25% higher than the Day 1 release prior to storage. In some embodiments, a hydride/vinyl ratio of >1:1 provides a Day 1 release after six months of storage at 25° C. and 60% relative humidity from approximately 10% lower to approximately 20% higher than the Day 1 release prior to storage. In some embodiments, a hydride/vinyl ratio of >1:1 provides a Day 1 release after six months of storage at 25° C. and 60% relative humidity from approximately 5% lower to approximately 15% higher than the Day 1 release prior to storage. In some embodiments, a hydride/vinyl ratio of >1:1 provides a Day 1 release after six months of storage at 25° C. and 60% relative humidity from approximately 2% lower to approximately 19% higher than the Day 1 release prior to storage. In some embodiments, a hydride/vinyl ratio of >1:1 provides a Day 1 release after six months of storage at 25° C. and 60% relative humidity from approximately 1% to approximately 15% higher than the Day 1 release prior to storage. In some embodiments, a hydride/vinyl ratio of >1:1 provides a Day 1 release after six months of storage at 25° C. and 60% relative humidity from approximately 1% to approximately 10% higher than the Day 1 release prior to storage. Thus, and unexpectedly, some hydrosilation of EE appears to be necessary in order to achieve an acceptable EE release profile over the course of the four quarterly product-use cycles or the 364-day product-use period.

What is more, it has been surprisingly discovered that when using NuSil™ MED4-4224, a 10:1 ratio of component parts A and B must have a narrow range of hydride/vinyl ratio to obtain consistent release of EE throughout the four quarterly product-use cycles or the 364-day product-use period. In certain embodiments, this hydride/vinyl ratio can be from approximately 1:2 to approximately 5:1. In some embodiments, the hydride/vinyl ratio is from approximately 1:1.5 to approximately 4:1. In some embodiments, the hydride/vinyl ratio is from approximately 1:1.5 to approximately 3:1. In some embodiments, the hydride/vinyl ratio is from approximately 1:1.5 to approximately 2:1. In some embodiments, the hydride/vinyl ratio is from approximately 1:1.5 to approximately 1.5:1. In some embodiments, the hydride/vinyl ratio is from 1:1 to 1.3:1. In some embodiments, the hydride/vinyl ratio is from 1:1 to 1.2:1. Hydride to vinyl ratio can be adjusted by specifying the amount of vinyl-terminated dimethylsiloxanes and dimethylsilicones in the pre-cured elastomer when ordering.

As previously discussed, there are additional factors that contribute to the amount of EE that is released from the system on Day 1 of each product-use cycle. Particle size of the EE influences the rate at which the compound solubilizes into the core and ultimately affects the release profile of the drug from the system. In addition, it was surprisingly discovered that the temperature and relative humidity at which the EE-containing core is cured affects the amount of EE released on Day 1. Cure temperatures of 120° C. provided unacceptably excessive release amounts. Humidity levels also caused unpredictable effects as certain cure temperatures required lower relative humidity to ensure an acceptable amount of EE release on Day 1.

The combination of particle size, conditions at which the core is cured, and hydride/vinyl ratio in the ring body elastomer all contribute to the rate of EE release from the vaginal system over the four quarterly product-use cycles or over the 364-day product-use period and also contribute to the system's stability over extended periods of time. Thus, each of these factors must be harmonized to ensure a proper release profile over the four quarterly product-use cycles or over the 364-day product-use period and to ensure proper long-term stability. Too much hydride within the ring body elastomer reduces the amount of EE that is available in the system, particularly after long-term storage. Conversely, too little hydride, high cure temperatures, and high humidity during core curing provides excessively high bursts of EE on Day 1.

The vaginal system disclosed herein is reusable for four quarterly product-use cycles or usable for one 364-day produce-use period and is sufficiently stable for at least 18 months of storage at 25° C. and at 60% relative humidity. In certain embodiments, approximately 80 to approximately 95% of EE incorporated into the system during manufacture can be recovered from the system after approximately 6 to approximately 18 months of storage at a temperature of 25° C. and at 60% relative humidity. In some embodiments, approximately 81 to approximately 94% of EE incorporated into the system during manufacture can be recovered from the system after approximately 6 to approximately 18 months of storage at a temperature of 25° C. and at 60% relative humidity. In some embodiments, approximately 82 to approximately 93% of EE incorporated into the system during manufacture can be recovered from the system after approximately 6 to approximately 18 months of storage at a temperature of 25° C. and at 60% relative humidity. In some embodiments, approximately 83 to approximately 92% of EE incorporated into the system during manufacture can be recovered from the system after approximately 6 to approximately 18 months of storage at a temperature of 25° C. and at 60% relative humidity. In some embodiments, approximately 84 to approximately 91% of EE incorporated into the system during manufacture can be recovered from the system after approximately 6 to approximately 18 months of storage at a temperature of 25° C. and at 60% relative humidity. In some embodiments, approximately 85 to approximately 90% of EE incorporated into the system during manufacture can be recovered from the system after approximately 6 to approximately 18 months of storage at a temperature of 25° C. and at 60% relative humidity.

In certain embodiments, approximately 80 to approximately 90% of EE incorporated into the system during manufacture can be recovered from the system after approximately 6, approximately 7, approximately 8, approximately 9, approximately 10, approximately 11, approximately 12, approximately 13, approximately 14, approximately 15, approximately 16, approximately 17, or approximately 18 months of storage at a temperature of 25° C. and at 60% relative humidity. In particular embodiments, approximately 80 to approximately 90% of the EE incorporated into the system during manufacture can be recovered from the system after approximately 6, approximately 12, and/or approximately 18 months of storage at a temperature of 25° C. and at 60% relative humidity. In some embodiments, approximately 80 to approximately 90% of EE incorporated into the system during manufacture can be recovered from the system after approximately 6 to approximately 9 months of storage at a temperature of 25° C. and at 60% relative humidity. In some embodiments, approximately 80 to approximately 90% of EE incorporated into the system during manufacture can be recovered from the system after approximately 6 to approximately 12 months of storage at a temperature of 25° C. and at 60% relative humidity. In some embodiments, approximately 80 to approximately 90% of EE incorporated into the system during manufacture can be recovered from the system after approximately 6 to approximately 15 months of storage at a temperature of 25° C. and at 60% relative humidity. In some embodiments, approximately 80 to approximately 90% of EE incorporated into the system during manufacture can be recovered from the system after approximately 6 to approximately 18 months of storage at a temperature of 25° C. and at 60% relative humidity. In some embodiments, approximately 80 to approximately 90% of EE incorporated into the system during manufacture can be recovered from the system after approximately 12 to approximately 15 months of storage at a temperature of 25° C. and at 60% relative humidity. In some embodiments, approximately 80 to approximately 90% of EE incorporated into the system during manufacture can be recovered from the system after approximately 12 to approximately 18 months of storage at a temperature of 25° C. and at 60% relative humidity. In some embodiments, approximately 80 to approximately 90% of EE incorporated into the system during manufacture can be recovered from the system after approximately 15 to approximately 18 months of storage at a temperature of 25° C. and at 60% relative humidity. In still further embodiments, approximately 80 to approximately 90% of EE incorporated into the system during manufacture can be recovered from the system after approximately 18 to approximately 24 months of storage at a temperature of 25° C. and at 60% relative humidity. In yet another embodiment, approximately 80 to approximately 90% of EE incorporated into the system during manufacture can be recovered from the system after approximately 24 to approximately 30 months of storage at a temperature of 25° C. and at 60% relative humidity. And in yet another embodiment, approximately 80 to approximately 90% of EE incorporated into the system during manufacture can be recovered from the system after approximately 24 to approximately 36 months of storage at a temperature of 25° C. and at 60% relative humidity.

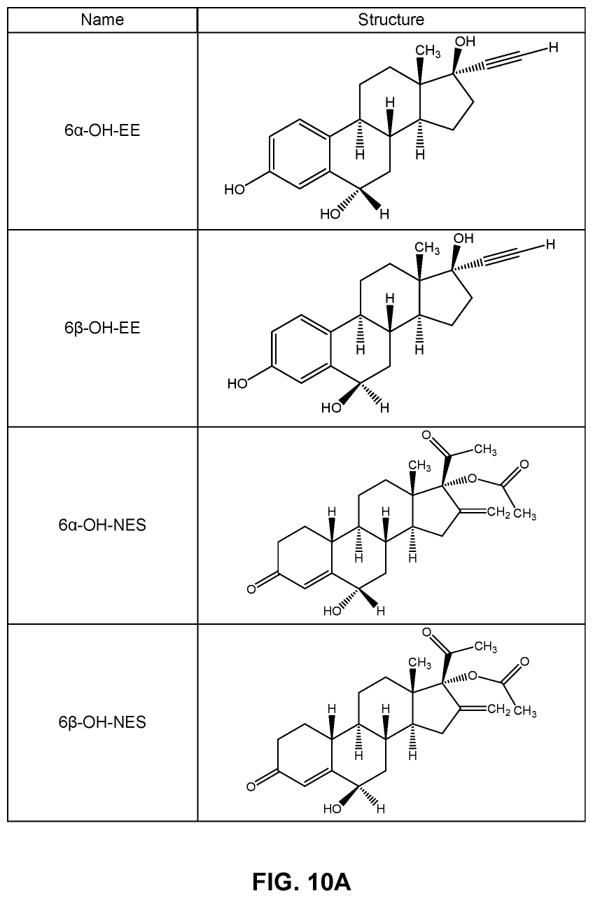

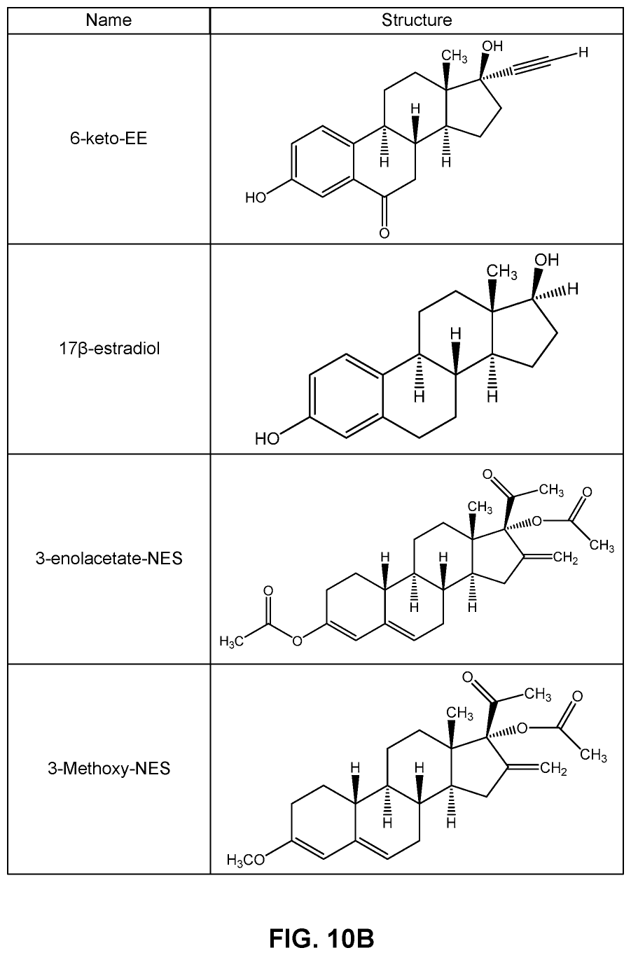

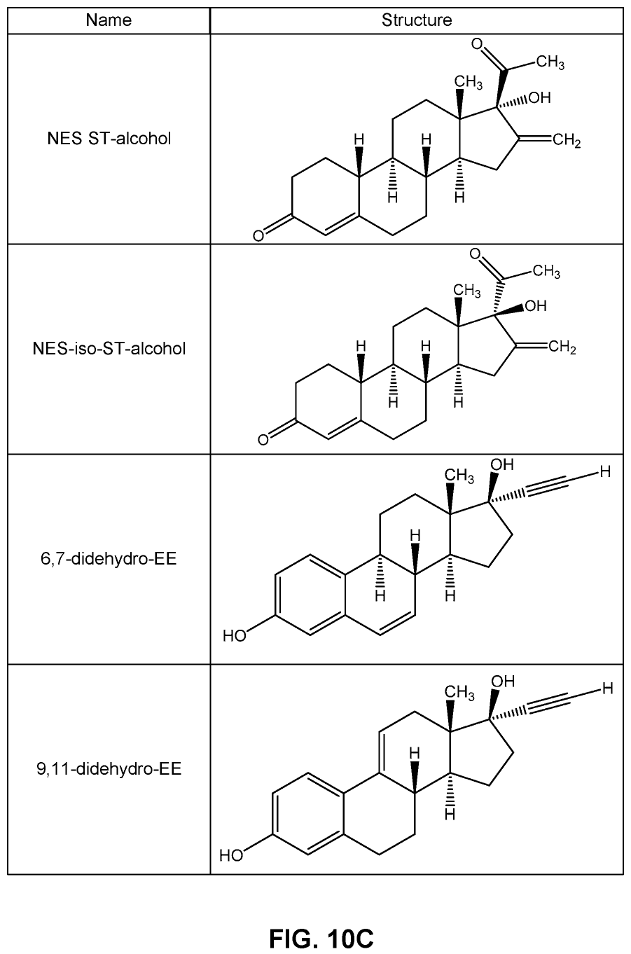

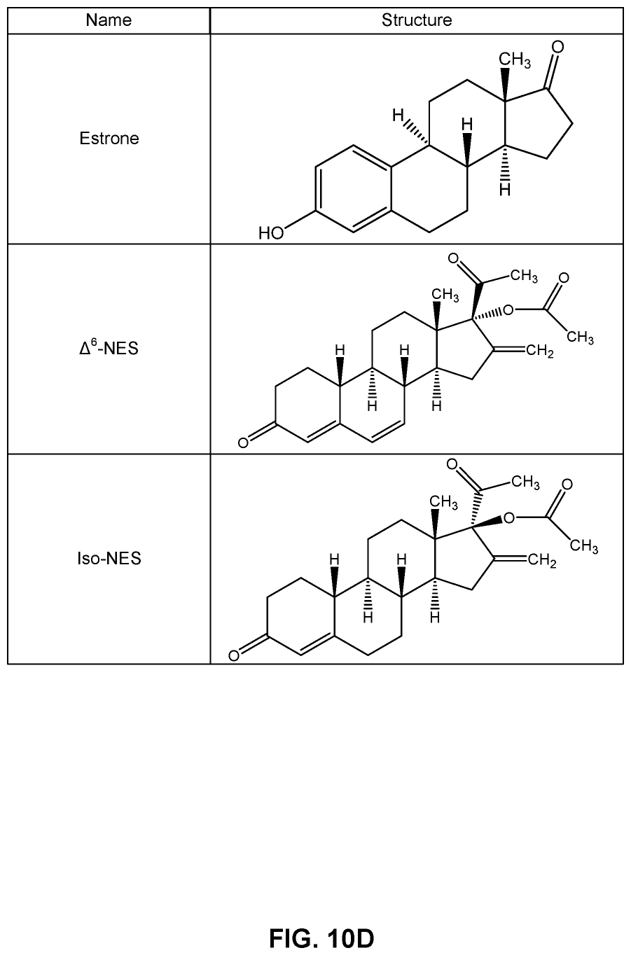

Although EE reaction with unreacted hydrosilane is believed to be responsible for the majority of unrecovered EE over any of the periods of time specified above, both EE and SA are susceptible to degradation over any of the periods of time noted above. As a result, the ring body and cores can contain a certain quantity of degradation products including, but not limited to, 6α-OH-EE, 6β-OH-EE, 6α-OH-NES, 6β-OH-NES, 17β-estradiol, NES ST-alcohol, NES iso-ST-alcohol, 6,7-didehydro-EE & 9,11-didehydro-EE, estrone, Δ⁶-NES, Iso-NES, 3-enolacetate-NES, and 3-methoxy-NES. Structures of these compounds are shown in FIGS. 10A-10D. That said, and in certain embodiments, the total percentage of EE and SA degradation products after 18 months of storage is detectible but not more than 5% as measured by HPLC (i.e. Liquid Chromatography Area Percent or “LCAP”). In certain embodiments, the total percentage of EE and SA degradation products after 18 months of storage is detectible but not more than 4 LCAP. In some embodiments, the total percentage of EE and SA degradation products after 18 months of storage is detectible but not more than 3 LCAP. In some embodiments, the total percentage of EE and SA degradation products after 18 months of storage is detectible but not more than 2 LCAP. In some embodiments, the total percentage of EE and SA degradation products after 18 months of storage is detectible but not more than 1 LCAP. Example 8 describes the procedure for determining the percentage of degradation products.

In certain embodiments, the total percentage of EE and SA degradation products after 24 months of storage is detectible but not more than 5 LCAP. In certain embodiments, the total percentage of EE and SA degradation products after 24 months of storage is detectible but not more than 4 LCAP. In some embodiments, the total percentage of EE and SA degradation products after 24 months of storage is not more than 3 LCAP. In some embodiments, the total percentage of EE and SA degradation products after 24 months of storage is detectible but not more than 2 LCAP. In some embodiments, the total percentage of EE and SA degradation products after 24 months of storage is detectible but not more than 1 LCAP.

In certain embodiments, the total percentage of EE and SA degradation products after 36 months of storage is detectible but not more than 5 LCAP. In certain embodiments, the total percentage of EE and SA degradation products after 36 months of storage is detectible but not more than 4 LCAP. In some embodiments, the total percentage of EE and SA degradation products after 36 months of storage is detectible but not more than 3 LCAP. In some embodiments, the total percentage of EE and SA degradation products after 36 months of storage is detectible but not more than 2 LCAP. In some embodiments, the total percentage of EE and SA degradation products after 36 months of storage is detectible but not more than 1 LCAP. The embodiments described herein minimize the amount of impurities contained within the vaginal system after approximately 18 to approximately 36 months of storage.

The vaginal system described herein is further detailed with reference to the examples shown below. These examples are provided for the purpose of illustration only and the embodiments described herein should in no way be construed as being limited to these examples. Rather, the embodiments should be construed to encompass any and all variations which become evident as a result of the teachings provided herein.

### EXAMPLES

### Example 1: XRPD Studies

XRPD patterns were collected with a PANalytical X'Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. Core samples were prepared for analysis by slicing into thin disks using a razor blade. A specimen of the sample was sandwiched between 3-μm-thick films and analyzed in transmission geometry. A beam-stop, short antiscatter extension, antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data acquisition parameters for each pattern are displayed above the image in the Data section of this report including the divergence slit (DS) before the mirror. XRPD patterns were obtained in the 20 range ˜7°-26°.

Figures labeled “Image by PatternMatch v3.0.4” were generated using unvalidated software.

### Example 2: Manufacture of EE 1302 Silicone Elastomer Samples for NMR Studies

MED4-4224 (previously known as DDU-4331) was supplied by NuSil™ Technology LLC (Carpinteria, CA, USA). Non-micronised 17α-ethinyl-¹³C₂-estradiol (20,21-¹³C₂ labelled; 99.1% isotopic enrichment) (EE-¹³C₂) was purchased from Cambridge Isotope Laboratories, Inc. (Andover, MA, USA). Particle size reduction of EE-¹³C₂ was achieved by manual grinding in a mortar and pestle.

Silicone elastomer mixes without EE were prepared by intimately mixing Part A and Part B (9:1) in a DAC150 FVK-Z Speedmixer™ (3000 rpm, 30 s). EE-¹³C₂-loaded (2% w/w) silicone elastomer mixes were similarly prepared except with extended speedmixing at 3000 rpm for 60 seconds to achieve a dispersion of the drug powder in the silicone elastomer. The elastomer mix was poured onto glass plates fitted with a cellulose acetate release liner and 1 mm spacers. After pouring, a second acetate release liner and glass plate were set on top and the mixture compressed to form thin viscous films. Non-medicated silicone elastomer samples were cured in an oven at 150° C. for 10 min. Despite adjustments to the cure conditions (final temperature 130° C. for >20 h), the EE-¹³C₂ loaded silicone samples only partly cured to form gum-like consistency materials due to EE inhibition of the curing reaction.

### Example 3: Solvent Extraction of EE from Cured EE-¹³C₂ Silicone Elastomer Samples

To increase detection sensitivity for any bound EE using ¹³C solid state NMR, the non-bound EE fraction was extracted from the silicone elastomer sample. The elastomer samples were placed in individually labeled glass vials. CDCl₃ or acetone (10-40 mL, depending on EE loading) was added to each extraction flask. Flasks were sealed and stored at ambient temperature for 24 hours with periodic manual shaking. This extraction protocol was repeated three times using fresh volumes of solvent to ensure complete extraction of the non-bound EE. The elastomer samples were removed from the solvent and dried overnight by solvent evaporation in preparation for ¹³C solid state NMR analysis.

### Example 4: NMR Spectra of Silicone Elastomer Samples Containing EE-¹³C₂

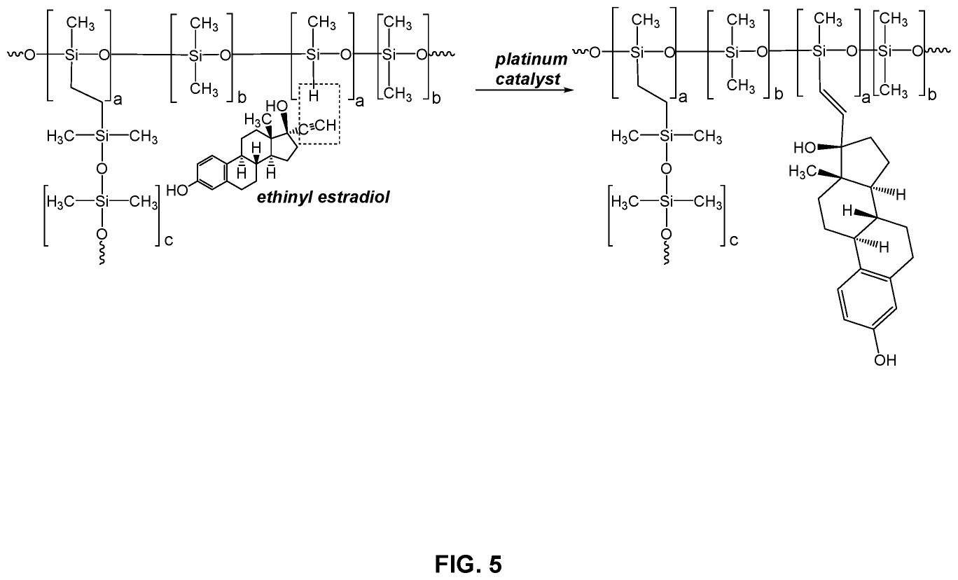

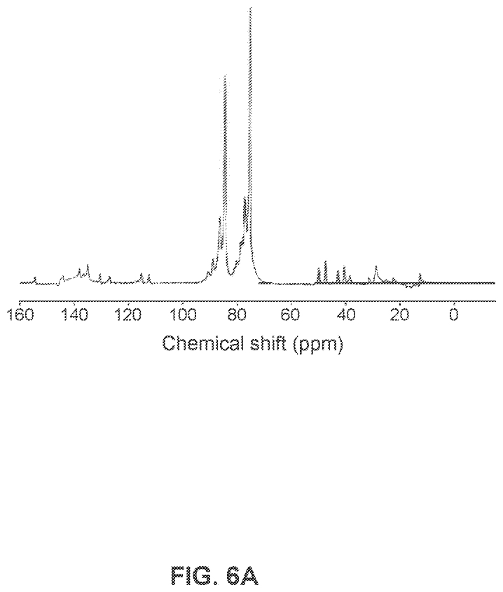

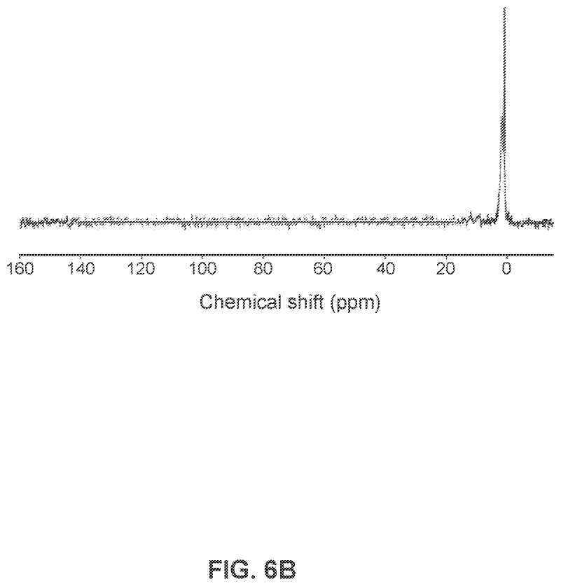

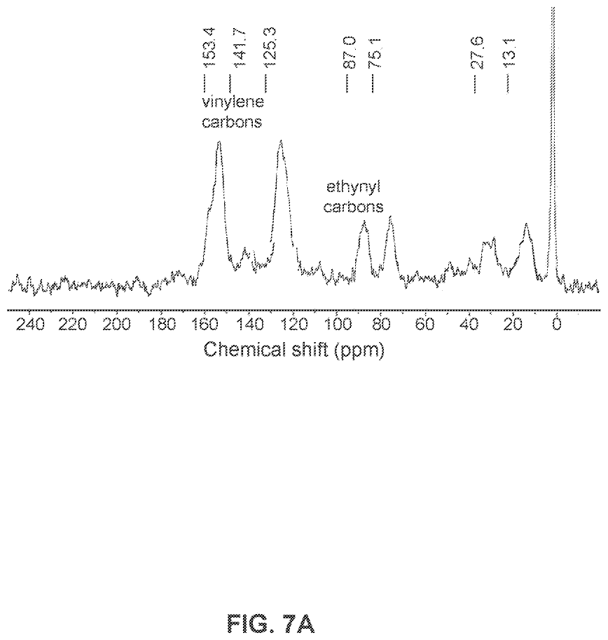

FIG. 7A shows the ¹³C-solid state NMR spectra for an EE-¹³C₂ silicone sample before solvent extraction. The chemical shifts associated with the ¹³C-labelled ethynyl groups are visible at 75 and 87 ppm. A second set of intense signals are observed at 125 and 153 ppm. These signals at 125 and 153 ppm are not observed in the EE-¹³C₂ or elastomer reference spectra (FIGS. 6A and 6B, respectively) and are attributed to newly-formed vinylene carbons produced from the hydrosilylation reaction between the ethynyl groups of the EE-¹³C₂ and the hydrosilane groups within the silicone elastomer (FIG. 4). Analysis of the EE-¹³C₂ plus elastomer material following acetone extraction showed that the ethynyl signals (75 and 87 ppm) associated with the non-bound EE-¹³C₂ were no longer visible in the post-extraction sample (FIG. 7B), confirming that the non-bound EE-¹³C₂ fraction had been successfully removed via solvent extraction. More interestingly, the new vinylene signals at 125 and 153 ppm were still observed and showed no reduction in intensity when compared to the non-extracted sample (FIG. 7A), clearly indicating that they could not be removed from the silicone elastomer by solvent extraction and therefore must be attributed to bound EE. Therefore, FIGS. 7A and 7B provide direct evidence for the formation of the irreversible covalent bond between the ethynyl groups of the EE-¹³C₂ and the hydrosilane groups of the addition-cure silicone elastomer.

### Example 5: Tensile Strength and Elongation Testing

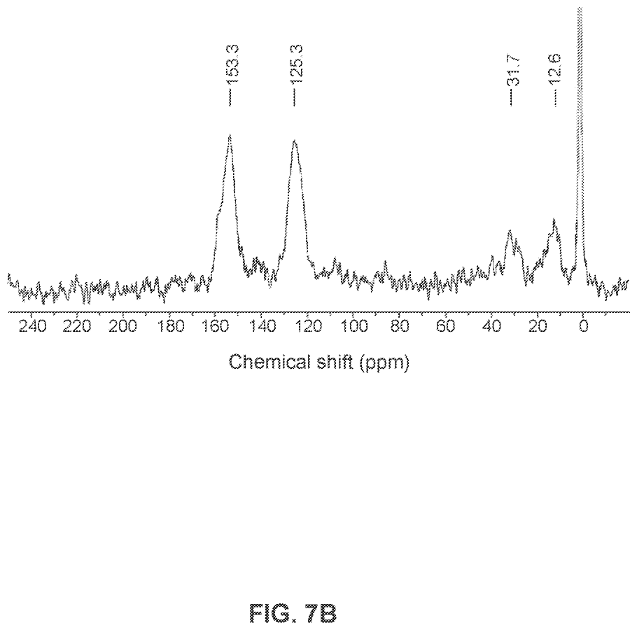

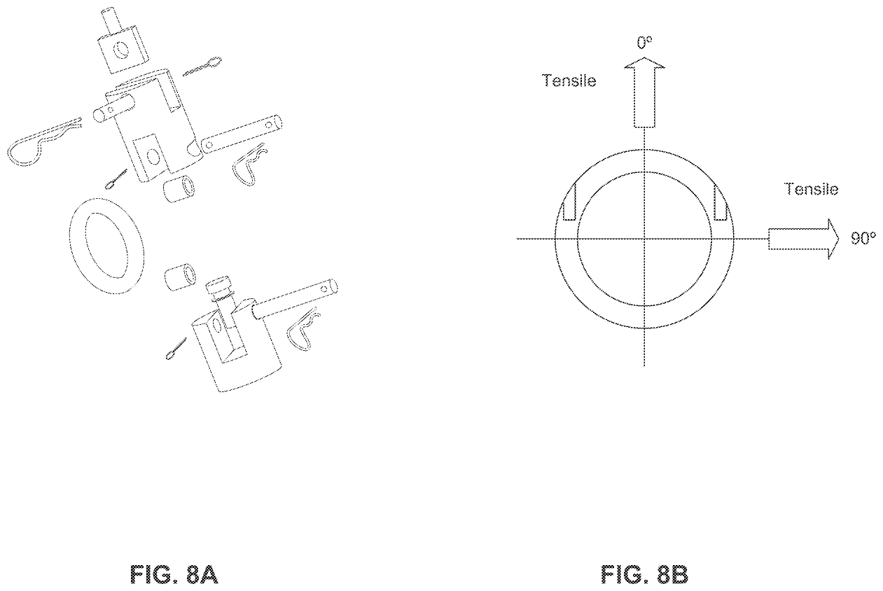

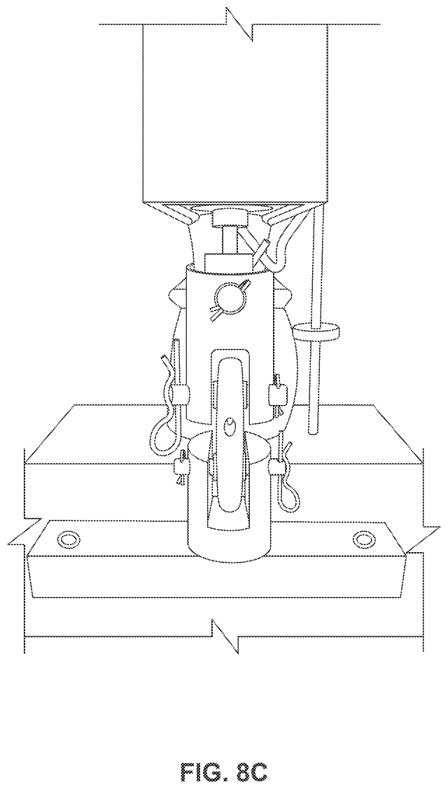

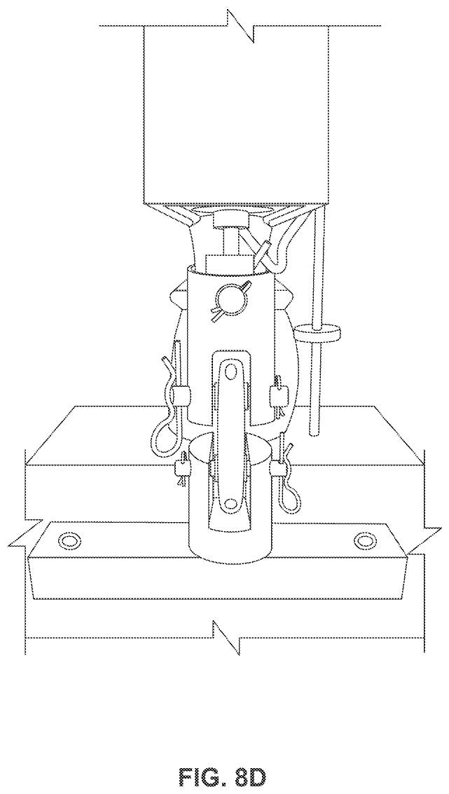

Tensile strength and elongation testing were performed on a calibrated Stable Micro Systems TA.XTPlus texture analyzer equipped with a TEXTURE1-1 tensile rig (FIG. 8A) using a Texture Exponent 32 software program and a 50 kg (PL/CEL5) load cell. The instrument parameters used for tensile strength testing are shown in Table 1.

TABLE 1
Instrument Settings for Tensile Strength and Elongation Analysis

| Parameters | Settings | |
|---|---|---|
| T.A. settings | Cycle Until Count (Distance) | |
| Test Mode | Tension | |
| Load Cell | 50 | kg |
| Test Speed | 8.5 | mm/sec |
| Target Mode | Distance | |
| Distance | 400 | mm |
| Trigger Type | Auto (Force) | |
| Trigger Force | 10 | g |

| | |
|---|---|
| Number of Measurements Per Ring | 1 |
| Number of Samples (Rings) | 10 |
| Temperature | Ambient |

Ring bodies that did not contain cores were equilibrated to room temperature prior to testing. The cross-sectional diameter and internal diameter of 10 rings were measured for calculation purposes.

Ten rings were measured parallel to the core channels, along the 0° line and ten additional rings were measured perpendicular to the core channels, along the 90° line (FIG. 8B). The instrument was started, and a 50 kg load cell was mounted on the instrument according to the procedure described in the instrument instruction manual, ensuring that the correct screws were used to attach the black rig holder to the instrument. The screws were at least 30 mm long and went completely into the countersunk holes in the rig holder (FIG. 8C). A force calibration and/or daily check were conducted according to the procedures described in the instruction manual. The upper rig was lowered to a position just above the lower rig, ensuring that the upper and lower rig were aligned. Height calibration was performed according to the procedure described in the instrument instruction manual.

For measurements parallel to the cores, a single ring was placed in the upper and lower rig according to machine instructions, with the channel opening pointing upwards. One channel opening was visible on each side of the upper rig (FIG. 8C). The measurement was performed according to instrument instructions and the process was repeated on the remaining nine rings.

For measurements perpendicular to the cores, a single ring was place in the upper and lower rig according to machine instructions, with the channel opening pointing outwards, towards the operator. Both channel openings were visible in the set-up (FIG. 8D). The measurement was performed according to instrument instructions and the process was repeated on the remaining nine rings.

Tensile strength, σ, was calculated for each ring according to the formula:

$$σ = ( F \times 4 ) \div ( 2 \times π \times {d}^{2} )$$

wherein F is the breaking force (N) and d is the average cross-sectional diameter of the ring body (mm) measured for 10 rings.

Internal circumference of the ring, Cᵢₙₜ (nm) was calculated according to the formula:

$${C}_{i n t} = {d}_{i} \times π$$

wherein dᵢ is the average internal diameter of the ring (mm) measured from 10 rings as described herein.

Elongation at break, E, is calculated for each ring according to:

$$E = ( 2 l + 2 r + {C}_{roll} - {C}_{i n t} ) / {C}_{i n t} \times 100$$

wherein:
l is the final distance between upper and lower rig (mm);
r is the distance between the center of the rollers at height calibration (15 mm);
Cᵣₒₗₗ is the circumference of the rollers (47 mm); and
Cᵢₙₜ is the internal circumference of the ring (mm).

Results of tensile strength testing are shown in Table 2. Results from elongation studies are shown in Table 3.

TABLE 2
Tensile Strength Testing Results

| | | | Cross- | Tensile | Tensile |
|---|---|---|---|---|---|
| | | | Sec- | Strength (σ) | Strength (σ) |
| | F (N) | F (N) | tional | Perpendicular | Parallel |
| Ring | Perpendicular | Parallel | Area | to Cores | to Cores |
| No. | to Cores | to Cores | (mm²) | (N/mm²) | (N/mm²) |
| 1 | 353.327 | 340.608 | 54.60 | 9645.6 | 9298.3 |
| 2 | 398.655 | 319.397 | 54.60 | 10883.0 | 8719.3 |
| 3 | 399.192 | 368.062 | 54.60 | 10897.6 | 10047.8 |
| 4 | 395.33 | 343.358 | 54.60 | 10792.2 | 9373.4 |
| 5 | 397.763 | 322.092 | 54.60 | 10858.6 | 8792.9 |
| 6 | 388.947 | 298.22 | 54.60 | 10618.0 | 8141.2 |
| 7 | 395.988 | 287.767 | 54.60 | 10810.2 | 7855.8 |
| 8 | 374.297 | 378.507 | 54.60 | 10218.0 | 10333.0 |
| 9 | 348.705 | 311.893 | 54.60 | 9519.4 | 8514.4 |
| 10 | 346.007 | 441.245 | 54.60 | 9445.7 | 12045.7 |
| Mean | 379.821 | 341.115 | 54.60 | 10368.8 | 9312.2 |
| Min | 346.007 | 287.767 | 54.60 | 9445.7 | 7855.8 |
| Max | 399.192 | 441.245 | 54.60 | 10897.6 | 12045.7 |
| SD | 22.31 | 45.37 | 0.00 | 609.15 | 1238.67 |

TABLE 3
Elongation Testing Results

| | l (mm) | E (%) | l (mm) | E (%) |
|---|---|---|---|---|
| Ring | Parallel | Parallel | Perpendicular | Perpendicular |
| No. | to Cores | to Cores | to Cores | to Cores |
| 1 | 289.052 | 420.2 | 301.614 | 440.2 |
| 2 | 279.900 | 405.7 | 324.448 | 476.4 |
| 3 | 301.707 | 440.3 | 320.150 | 469.6 |
| 4 | 288.697 | 419.7 | 319.627 | 468.8 |
| 5 | 277.890 | 402.5 | 328.794 | 634.6 |
| 6 | 271.784 | 392.8 | 319.377 | 468.4 |
| 7 | 259.451 | 373.2 | 322.550 | 473.4 |
| 8 | 304.030 | 444.0 | 306.151 | 447.4 |
| 9 | 273.986 | 396.3 | 292.725 | 426.1 |
| 10 | 331.482 | 487.6 | 296.655 | 432.3 |
| Mean | 287.798 | 418.2 | 313.209 | 473.7 |
| Min | 259.451 | 373.2 | 292.725 | 426.1 |
| Max | 331.482 | 487.6 | 328.794 | 634.6 |
| SD | 20.49 | 32.54 | 12.74 | 59.41 |

### Example 6: Fatigue Testing

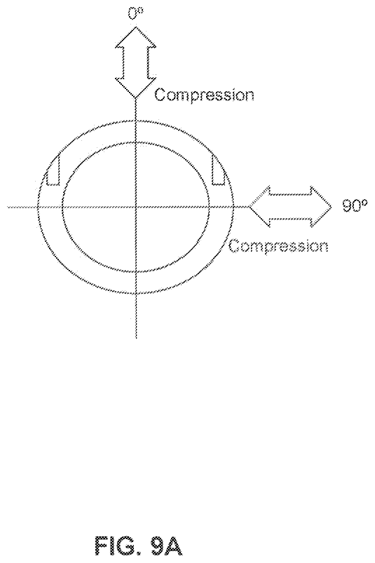

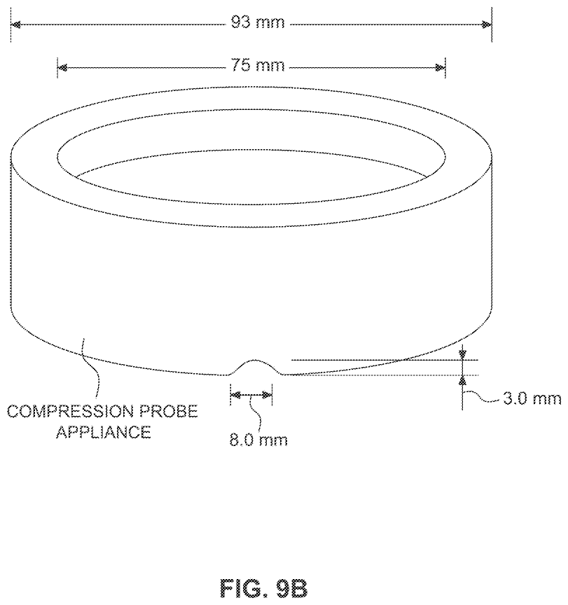

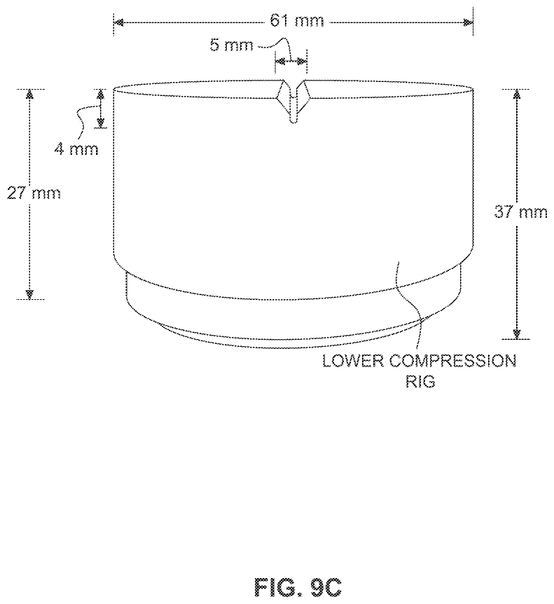

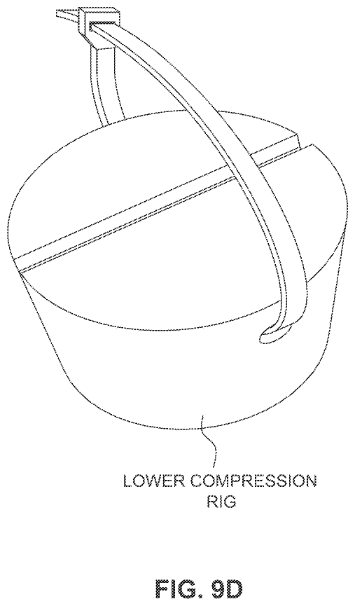

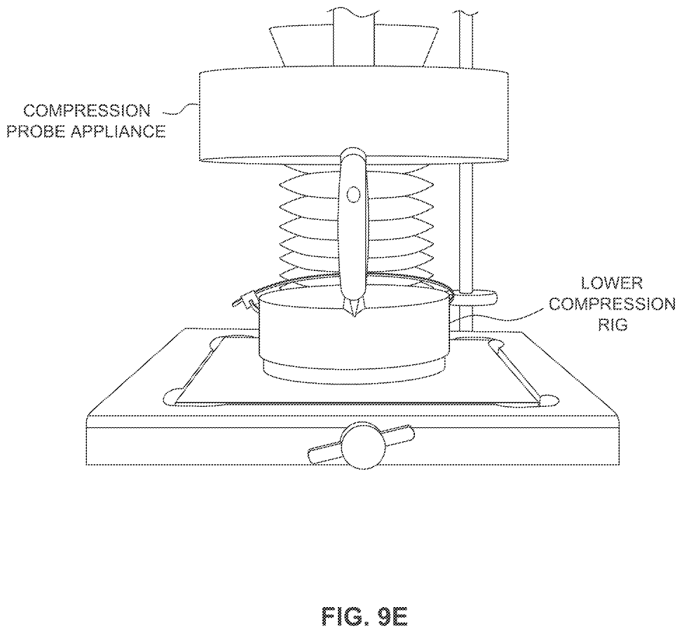

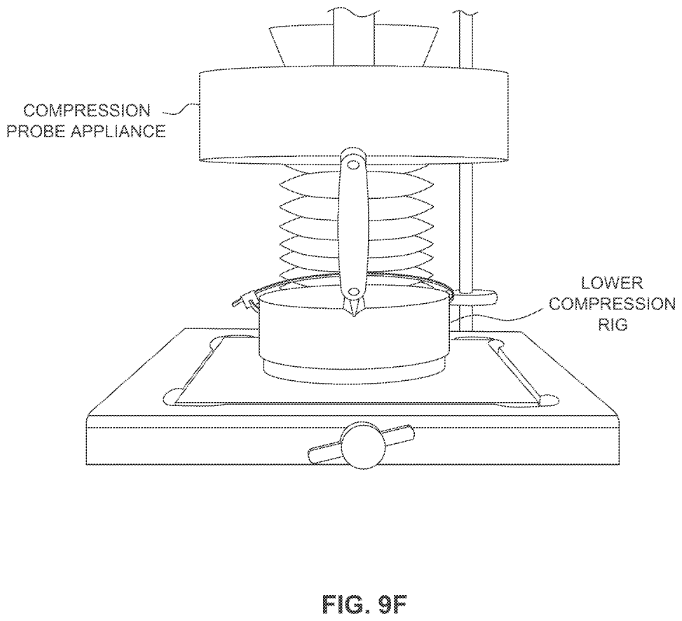

Compression force, fatigue, and seal integrity testing were performed on a calibrated Stable Micro Systems TA.XTPlus texture analyzer equipped with a TEXTURE1-2 compression rig with a 9 mm slit and a lower compression rig with a 202 mm×4.8 mm nylon strap (FIGS. 9A, 9B, and 9C). A 5 kg (PL/CEL5) load cell, a 75 mm (SMS P/75) compression probe, and a heavy-duty platform (HDP/90) were used, in addition to Texture Exponent 32 software. The instrument parameters used for compression analysis are shown in Table 4.

TABLE 4
Instrument Settings for Compression Analysis

| Parameter | Setting | |
|---|---|---|
| T.A. Setting | Cycle Until Count (Distance) | |
| Test Mode | Compression | |
| Load Cell | 5 | kg |
| Test Speed | 40.0 | mm/sec |
| Pre-Test Speed | 2.0 | mm/sec |
| Target Mode | Distance | |
| Distance | 30.0 | mm |
| Trigger Force | 0.1 | N |
| Number of Measurements Per Ring | 1000 | |
| Number of Samples (Rings) | 10 | |
| Force Limit | 10 | N |
| Force Range | 50 | N |

| | |
|---|---|
| Temperature | Ambient |

Ring bodies that did not contain cores were equilibrated to room temperature at least three hours prior to testing. Ten rings were measured parallel to the core channels, along the 0° line and ten additional rings were measured perpendicular to the core channels, along the 90° line (FIG. 9D). The instrument was started, and a 5 kg load cell was mounted on the instrument. The compression rig was mounted according to the instrument instruction manual. A calibration and/or daily check was performed according to the instrument instruction manual. The compression probe was lowered to just above the lower rig to make sure the slits in the probe appliance and the lower rig were aligned. Alternatively, the heavy-duty platform was adjusted to ensure alignment.

For measurement parallel to the cores, a single ring was mounted as shown in FIG. 9E and secured, with the channel opening pointing upwards and the ring fitted in the slits. The ring was secured by the strap, but it was possible to rotate it. One channel opening was visible on each side of the compression probe. It was important that the ring was mounted perpendicular to the rig. The compression plate was carefully lowered to just above the ring without compressing it. The measurement was performed, and the process was repeated on the remaining nine rings.

For measurement perpendicular to the cores, a single ring was mounted as shown in FIG. 9F, with the channel opening pointing outwards and the ring fitted in the slits. The ring was secured by the strap, but it was possible to rotate it. Both channel openings were visible in the set-up. It was important that the ring was mounted perpendicular to the rig. The compression probe was carefully lowered to just above the ring without compressing it. The measurement was performed, and the process was repeated on the remaining nine rings.

For each set of ten rings, the average force in Newton (N) for the 1^{st} compression and for the 1000^{th} compression was calculated.

Fatigue (percentage chain in compression force) due to cycle loading, ΔF_{c}, was calculated for each ring according to the formula:

$$Δ {F}_{c} = 100 \times {F}_{1000} \div {F}_{1}$$

wherein F₁ is the compression force for the 1^{st} compression and F₁₀₀₀ is the compression force for the 1000^{th} compression.

No impact on seal integrity for the tested rings was noted.

Results of the fatigue testing studies are shown in Table 5.

TABLE 5
Fatigue Testing Results

| | Force (N) | Force(N) After | | Force (N) | Force(N) After | |
|---|---|---|---|---|---|---|
| | After | 1000^{th} | | After | 1000^{th} | |
| | 1^{st}Compression | Compression | | 1^{st}Compression | Compression | |
| | (F₁) | (F₁₀₀₀) | | (F₁) | (F₁₀₀₀) | |
| Ring | Parallel | Parallel | ΔF_{c} | Perpendicular | Perpedicular | ΔF_{c} |
| No. | to Cores | to Cores | (%) | to Cores | to Cores | (%) |
| 1 | 4.723 | 4.468 | 94.6 | 3.907 | 3.839 | 98.3 |
| 2 | 4.746 | 4.523 | 95.3 | 3.875 | 3.799 | 98.0 |
| 3 | 4.770 | 4.508 | 94.5 | 3.845 | 3.768 | 98.0 |
| 4 | 4.747 | 4.464 | 94.0 | 3.953 | 3.862 | 97.7 |
| 5 | 4.534 | 4.319 | 95.3 | 3.951 | 3.900 | 98.7 |
| 6 | 4.691 | 4.479 | 95.5 | 3.888 | 3.837 | 98.7 |
| 7 | 5.046 | 4.768 | 94.5 | 3.955 | 3.870 | 97.9 |
| 8 | 4.723 | 4.457 | 94.4 | 3.777 | 3.693 | 97.8 |
| 9 | 4.661 | 4.467 | 95.8 | 3.801 | 3.730 | 98.1 |
| 10 | 4.799 | 4.577 | 95.4 | 3.993 | 3.926 | 98.3 |
| Mean | 4.744 | 4.503 | 94.9 | 3.895 | 3.822 | 98.1 |
| Min | 4.534 | 4.319 | 94.0 | 3.777 | 3.693 | 97.7 |
| Max | 5.046 | 4.768 | 95.8 | 3.993 | 3.926 | 98.7 |
| SD | 0.13 | 0.11 | 0.59 | 0.07 | 0.07 | 0.35 |

### Example 7: Extraction Procedure to Determine Recoverable EE and NES after Storage for any Period of Time

### Solutions:

Diluent: methanol/water 58/42 v/v
Dried NES and EE before weighing (100-105° C. 3 h)
EE stock solution: Dissolved 25.0 mg of EE and diluted to 250.0 mL with methanol (duplicates, EE₁ and EE₂)
NES stock solution: Dissolved 50.0 mg of NES and diluted to 100.0 mL with methanol (duplicates, NES₁ and NES₂)
Standard solutions:
   S1: Diluted 5.0 mL of NES₁ and 4.0 mL of EE₁ to 50.0 mL with diluent
   S2: Diluted 6.0 mL of NES₂ and 5.0 mL of EE₂ to 50.0 mL with diluent
   S3: Diluted 8.0 mL of NES₁ and 7.0 mL of EE₁ to 50.0 mL with diluent
   S4: Diluted 10.0 mL of NES₂ and 9.0 mL of EE₂ to 50.0 mL with diluent
System suitability solution (SST solution): Diluted 5.0 mL acetone+7.0 mL NES_{1+6.0} mL EE₁ to 50.0 mL with diluent.

### Extraction Procedure:

Rings were cut into 8 pieces and each piece was divided lengthwise then transferred to an Erlenmeyer flask.
140 mL of acetone (weight of flask was noted before and after acetone addition) was added to the flask. The flask was then capped.
The flask was shaken for 24 h at 180 rpm (weight was noted after extraction).
Diluted 2.5 mL of the extraction medium to 25.0 mL with diluent (test solution) and a sample was subsequently pulled for HPLC analysis.

### Liquid Chromatography

### Column

analytical column: Discovery C8, 5 μm, 150×4.6 mm (Supelco)
pre-column: Supelguard, Discovery C8, 5 μm, 20×4.0 mm (Supelco)
   stationary phase: endcapped C8 (5 μm particle size) USP L7
temperature: 30° ° C.
   Mobile phase: methanol/water 58/42, isocratic elution
   Flow rate: 1.2 mL/min
   Detection (assay): NES UV 240 nm, EE UV 280 nm
   Detection (identity): PDA (photodiode array detector) scanning 220-310 nm, NES 240 nm, EE 280 nm

### Injection: 20 μL

Run time: 15 min
System suitability: SST solution
Area precision (n=5): RSD (%)≤2.0
Peak tailing (T): 0.8≤T≤1.5
Blank injections: No interfering peaks
   Retention times: EE approximately 7 min and NES approximately 9 min
   Results Assay: Report the average value of three different rings and express as mg EE/ring and mg NES/ring.
   Results Content uniformity: Calculate the average value of ten different rings. Report with or without remarks according the guidelines outlined in the US Pharmacopeial Convention, incorporated herein by reference, and in particular USP <905>.
   Results Identity: If the retention time in the test and standard solution match in the assay and UV spectra of EE/NES in test solution and PDA library match report without remarks, otherwise with remarks.

### Example 8: Determination of SA and EE Degradation Products

### REFERENCES

Ethinylestradiol (EE), working reference standard
NESTORONE® (NES), working reference standard
17β-estradiol (structure shown in FIG. 10B)
Estrone estradiol (structure shown in FIG. 10D)
Δ6-NESTORONE® estradiol (structure shown in FIG. 10D)
NES ST-alcohol estradiol (structure shown in FIG. 10C)

### Reagents

Methanol, HPLC grade
Acetone, p.a.
Water, purified
Acetonitrile, gradient grade

### Solutions

Dry NES and EE before weighing (100-105° C. 3 h)
NES stock solution: Dissolved 75.0 mg of NES and diluted to 50.0 mL with methanol (duplicates, SSA1 and SSA2)
EE stock solution: Dissolved 15.0 mg of EE and diluted to 50.0 mL with methanol (duplicates, SSB1 and SSB2)
Standard Solutions:
S5: Diluted 5.0 mL of SSA1 and 5.0 mL of SSB1 to 50.0 mL with methanol
S4: Diluted 7.0 mL of S5 to 10.0 mL with methanol
S3: Diluted 2.5 mL of SSA2 and 2.5 mL of SSB2 to 50.0 mL with methanol
S2: Diluted 5.0 mL of S5 to 50.0 mL with methanol
S1: Diluted 5.0 mL of S3 to 50.0 mL with methanol
NES area reject stock solution: Diluted 2.5 mL SSA1 to 50.0 mL with methanol (R1).
EE area reject stock solution: Diluted 2.5 mL SSB1 to 50.0 mL with methanol (R2).
NES EE area reject solution: Diluted 1.0 mL R1+5.0 mL R2 to 100.0 mL with methanol. Rejection peak area at 254 nm: NES area. Rejection peak area at 280 nm: EE area.
System suitability solution:
SST1: Dissolved 15.0 mg 17β-estradiol and diluted to 50.0 mL with methanol
SST2: Dissolved 15.0 mg estrone and diluted to 50.0 mL with methanol
SST3: Dissolved 15.0 mg Δ⁶-NESTORONE® and diluted to 10.0 mL with methanol
SST4: Dissolved 15.0 mg NES ST-alcohol and diluted to 10.0 mL with methanol
SST solution: Dilute 2.5 mL SSA2+5.0 mL SSB2+5.0 mL SST1+5.0 mL SST2+2.5 mL SST3+2.5 mL SST4 to 50.0 mL with methanol.

### Extraction Procedure

Cut the ring in 8 pieces and divided each piece lengthwise, transfer to Erlenmeyer flask.
Added 70 mL of acetone (noted weight before and after), capped Erlenmeyer flask.
Shook for 24 h at 180 rpm (noted weight after extraction).
Transferred 10.0 mL of extraction medium to a test tube and evaporated to dryness.
Dissolved in 1.0 mL of methanol. When a clear upper phase was obtained, transferred to LC vial (test solution)

### Liquid Chromatography

### Column

analytical column: SUNFIRE™ C18, 5 μm, 250×4.6 mm (Waters)
pre-column: SUNFIRE™ C18, 5 μm, 20×4.6 mm (Waters)
stationary phase: reversed phase endcapped C18, 100 Å(5 μm), USP L1
temperature: 35° ° C.

### Mobile Phase A: Acetonitrile, B: Water

| Time (min) | Mobile Phase A (% v/v) | Mobile Phase B (% v/v) |
|---|---|---|
| 0 | 34 | 66 |
| 20 | 34 | 66 |
| 23 | 42 | 58 |
| 30 | 42 | 58 |
| 35 | 55 | 45 |
| 40 | 55 | 45 |
| 51 | 90 | 10 |
| 70 | 90 | 10 |
| 75 | 34 | 66 |
| 85 | 34 | 66 |

Flow rate: 1 mL/min

### Detection (UV): NES 254 nm, EE 280 nm

Detection (PDA): scanning 220-310 nm

### Injection: 10 UL

Sample temperature: 2-8° ° C.
Run time: 85 min
System suitability: SST solution
Resolution
≥2.0 between 17ß-estradiol and NES ST-alcohol at 280 nm
≥1.5 between EE and estrone at 280 nm
≥4.0 between A6-NESTORONE® and NES at 254 nm
Peak tailing (T): 0.8≤T≤1.5
Area precision (n=5): RSD (%)≤3.0 for NES peak at 254 nm, ≤3.0 for EE peak at 280 nm

| | | Relative Retention | |
|---|---|---|---|
| Related | Quantitation | Time (RRT) | RRT |
| substance | at [nm] | reference | 30° C. |
| 6α-OH-EE | 280 | 17β-estradiol | 0.28 |
| 6β-OH-EE | 280 | 17β-estradiol | 0.29 |
| 6-keto-EE | 254 | 17β-estradiol | 0.53 |
| 6α-OH-NES | 254 | 17β-estradiol | 0.60 |
| 6β-OH-NES | 254 | 17β-estradiol | 0.71 |
| 17β-estradiol | 280 | 17β-estradiol | 1.00 |
| NES ST- | 254 | 17β-estradiol | 1.04 |
| alcohol | | | |
| NES iso-ST- | 254 | 17β-estradiol | 1.06 |
| alcohol | | | |
| 6,7-didehydro- | 254 | 17β-estradiol | 1.15 |
| EE & 9,11- | | | |
| didehydro-EE | | | |
| Estrone | 280 | 17β-estradiol | 1.23 |
| Δ6-NES | 280 | NES | 0.96 |
| Iso-NES | 254 | NES | 1.13 |
| 3-enolacetate- | 254 | NES | 1.29 |
| NES | | | |
| 3-methoxy- | 254 | NES | 1.36 |
| NES | | | |

### Example 9: Condom Compatibility Testing

Five different types of condoms (three latex, one polyisoprene, and one polyurethane) were tested to determine if exposure to the vaginal ring system described herein would result in detrimental effects to the condom. Condom strength was measured using four different parameters: Force required for breakage (break force), percent condom elongation at breakage (percent elongation), pressure required to cause the condom to burst (burst pressure), and the condom's volume at burst (burst volume). A baseline was established for the analysis by measuring the four parameters of the condoms in their “as-received” condition with no exposure to heat, the ring system, or any lubricants (labeled “Baseline” in Table 6, below).

To test the compatibility of the vaginal system components with the condoms, an aqueous extract was prepared by placing 20 vaginal ring systems in a sufficient amount of water to test 20 condoms and agitating the resulting mixture for 24 hours. After the 24-hour period, the vaginal ring bodies were removed and the remaining extract was saturated with segesterone acetate and ethinyl estradiol. The amounts of segesterone acetate and ethinyl estradiol needed to ensure saturation were calculated by multiplying the solubilities of segesterone acetate and ethinyl estradiol in water at neutral pH (18.5 μg/mL and 11.0 μg/mL, respectively) by the volume of extract and adding an additional 20% of each agent.

Each type of condom was then covered with the saturated extract and conditioned at 40 ºC for 1 hour at which time the four parameters described above were measured (labeled “Sample” in Table 6). Two controls were also included: one where each condom was subjected to the same preparation and conditioning using water in place of the saturated extract (labeled “Control” in Table 6), and one where each condom was subjected to the same preparation and conditioning using mineral oil in place of the saturated extract (labeled “Mineral Oil” in Table 6).

As shown in Tables 6 and 7, the parameters of each condom in the “Sample” category measured within 10% of condoms in the “Control” group, illustrating that exposure to the components of the vaginal ring system did not cause detrimental effects to the condoms. In contrast, condoms exposed to the mineral oil lubricant became significantly weaker.

TABLE 6
Change in Condom Strength After Exposure to Vaginal Ring System Components

| | | | | | | Burst | | Burst | |
|---|---|---|---|---|---|---|---|---|---|
| | | | | | | Pressure in | | Volume in | |
| | | | | Percent | | Kilopascals | | Liters (L) | |
| | | Break Force in | | Elongation at | | (kPa) and | | and | |
| | | Newtons (N) | | Break (%) and | | Standard | | Standard | |
| | | and Standard | | Standard | | Deviation | | Deviation | |
| Condom | | Deviation (SD) | | Deviation (SD) | | (SD) | | (SD) | |
| Type | Exposure | (N) | SD | (%) | SD | (kPa) | SD | (L) | SD |
| Trojan ® | Baseline | 103.7 | 6.8 | 880.0 | 17.3 | 2.0 | 0.2 | 32.7 | 3.2 |
| Enz ® Latex | Control | 94.4 | 13.4 | 852.0 | 30.0 | 1.9 | 0.1 | 36.6 | 3.0 |
| (non- | Mineral | 4.7 | 1.1 | 326.0 | 85.0 | 0.9 | 17.7 | 0.1 | 9.8 |
| Lubricated) | Oil | | | | | | | | |
| | Sample | 89.7 | 8.9 | 875.0 | 25.6 | 1.9 | 0.1 | 35.8 | 2.3 |
| LifeStyles ® | Baseline | 86.3 | 9.7 | 792.0 | 21.0 | 2.3 | 0.2 | 38.5 | 3.1 |
| Latex | Control | 78.0 | 8.8 | 795.0 | 25.5 | 2.3 | 0.2 | 40.3 | 3.8 |
| (non- | Mineral | 12.0 | 5.0 | 542.0 | 138.1 | 1.1 | 0.2 | 27.6 | 6.7 |
| (Lubricated) | Oil | | | | | | | | |
| | Sample | 79.6 | 10.2 | 795.0 | 21.1 | 2.2 | 0.2 | 40.1 | 3.6 |
| Atlas ® | Baseline | 88.4 | 8.0 | 873.0 | 22.6 | 2.0 | 0.1 | 34.6 | 3.3 |
| Latex | Control | 85.9 | 6.1 | 893.0 | 15.4 | 2.0 | 0.1 | 36.7 | 3.8 |
| (non- | Mineral | 12.3 | 5.0 | 556.0 | 174.4 | 0.8 | 0.1 | 17.4 | 8.3 |
| Lubricated) | Oil | | | | | | | | |
| | Sample | 81.0 | 7.5 | 878.0 | 21.9 | 2.0 | 0.1 | 35.2 | 3.1 |
| LifeStyles ® | Baseline | 85.7 | 13.7 | 1033.0 | 31.5 | 1.8 | 0.1 | 46.8 | 2.8 |
| Skyn ® | Control | 80.9 | 10.5 | 1017.0 | 23.5 | 1.8 | 0.1 | 46.9 | 2.4 |
| Polyisoprene | Mineral | 24.6 | 9.6 | 781.0 | 131.5 | 0.7 | 0.0 | 13.9 | 2.1 |
| (Lubricated) | Oil | | | | | | | | |
| | Sample | 78.2 | 13.0 | 1013.0 | 29.5 | 1.7 | 0.2 | 45.3 | 3.8 |
| Trojan | Baseline | 46.1 | 10.8 | 539.0 | 17.1 | 9.8 | 0.9 | 7.6 | 0.6 |
| Supra ® | Control | 44.7 | 7.8 | 532.0 | 13.9 | 9.3 | 0.9 | 8.7 | 0.9 |
| Polyurethane | Mineral | 35.5 | 6.6 | 528.0 | 14.6 | 6.6 | 0.80 | 7.9 | 0.6 |
| (Lubricated) | Oil | | | | | | | | |
| | Sample | 45.6 | 12.1 | 537.0 | 20.2 | 9.1 | 0.6 | 8.6 | 0.6 |

TABLE 7
Percent Changes

| | Break | Elon- | Burst | Burst |
|---|---|---|---|---|
| | Force % | gation % | Pressure % | Volume % |
| | Change | Changes | Change | Change |
| | (sample | (sample | (sample | (sample |
| | vs. | vs. | vs. | vs. |
| Condom Type | control) | control) | control) | control) |
| Trojan ® Enz ® Latex | −5.0 | 2.7 | 0.0 | −2.3 |
| (non-Lubricated) | | | | |
| LifeStyles ® Latex | 2.1 | 0.0 | −2.4 | −0.5 |
| (non-(Lubricated) | | | | |
| Atlas ® Latex | −5.7 | −1.7 | −0.3 | −3.9 |
| (non-Lubricated) | | | | |
| LifeStyles ® Skyn ® | −3.3 | −0.4 | −3.9 | −3.4 |
| Polyisoprene | | | | |
| (Lubricated) | | | | |
| Trojan Supra ® | 2.0 | 0.9 | −2.2 | −1.5 |
| Polyurethane | | | | |
| (Lubricated) | | | | |

Percent change in Table 7 was calculated using the formula % change=100*(sample mean value−control mean value)/Control mean value

### Example 10: Pharmacodynamics

### Cardiac Electrophysiology

The effect of SA on the QTc interval was evaluated in a Phase 1 randomized, placebo and positive controlled, double-blind, single-dose, three-period, crossover thorough QTc study in 44 healthy adult female subjects. At the single intravenous bolus dose which produces 4.5-fold the therapeutic serum concentrations of SA achieved with the vaginal system, SA did not prolong the QTc interval to any clinically relevant extent.

### Example 11: Pharmacokinetics

### Absorption

The pharmacokinetics (PK) of the vaginal system described herein were determined during studies related to the prior approved use (i.e., 13 product-use cycles comprising a 21-day “product-in” period and a 7-day “product-out” period). Studies were conducted in 39 women who used the system for up to the 13 product-use cycles. Following vaginal administration, SA and EE were absorbed into systemic circulation with median Tₘₐₓ of approximately 2 hours in product-use cycle 1, product-use cycle 3, and product-use cycle 13. Concentrations of both components declined after Tₘₐₓ and became more constant after 96 hours post-dose. Over subsequent product-use cycles, the peak serum concentrations of SA and EE declined. PK parameters are summarized in Table 8 and Table 9.

TABLE 8
Mean (SD) PK Parameters for SA following Administration

| | AUC_{0-21 day} | | AUC_{0-1 day} | Cₘₐₓ | | C_{avg} | |
|---|---|---|---|---|---|---|---|
| Cycle | (pmol*hr/L) | | (pmol*hr/L) | (pmol/L) | | (pmol/L) | |
| 1 | 259,740 | (45,630) | 40,500 (8,640) | 3,097 | (850) | 516 | (92) |
| 3 | 480,330 | (108,000) | 36,450 (4,320) | 980 | (359) | 353.7 | (78) |
| 13 | 127,440 | (27,270) | 10,530 (3,780) | 794 | (313) | 254 | (54) |

TABLE 9
Mean (SD) PK Parameters for EE following Administration

| | AUC_{0-21 day} | AUC_{0-1 day} | Cₘₐₓ | | C_{avg} | |
|---|---|---|---|---|---|---|
| Cycle | (pmol*hr/L) | (pmol*hr/L) | (pmol/L) | | (pmol/L) | |
| 1 | 74,814 (33,026) | 7,077 (2,359) | 435 | (131) | 148 | (64) |
| 3 | 49,539 (15,839) | 3,033 (1,348) | 202 | (108) | 98 | (30) |
| 13 | 32,352 (13,817) | 2,359 (1,011) | 131 | (54) | 64 | (27) |

The volume of distribution of SA is 19.6 L/kg. SA is approximately 95% bound to human serum proteins and has negligible binding affinity for sex hormone-binding globulin (SHBG). EE is highly protein bound but not specifically bound to serum albumin (98.5%) and induces an increase in the serum concentrations of SHBG.

### Metabolism

In vitro data show that both SA and EE are metabolized by the cytochrome P450 (CYP) 3A4 isoenzyme. In human serum, two oxidative metabolites (5α-dihydro-and 17α-hydroxy-5αdihydro metabolites) constitute 50% of exposure relative to SA. Both metabolites are not considered as active metabolites with EC50 to progesterone receptor 10-fold higher than that of SA. EE is primarily metabolized by aromatic hydroxylation, but a wide variety of hydroxylated and methylated metabolites are formed. These are present as free metabolites and as sulfate and glucuronide conjugates. The hydroxylated EE metabolites have weak estrogenic activity.

### Excretion

The mean (SD) half-life of SA is 4.5 (3.4) hours. EE is known to be excreted in the urine and feces as glucuronide and sulfate conjugates, and it undergoes enterohepatic recirculation. The mean (SD) half-life of EE is 15.1 (7.5) hours.

The in vitro studies suggest that SA is unlikely to inhibit or induce CYP enzymes at the therapeutic dose.

### Example 12: Segesterone Acetate and Ethinyl Estradiol Levels with a Regression Model of 364-Day Continuous Use

Continuous use models for the vaginal ring system were post hoc analyses based on PK data obtained from a phase 1 PK sub-study.

Modeling from the PK Study Data

### Clinical Study Design

During a phase 3 trial of the vaginal ring system, three centers participated in a PK sub-study. The PK sub-study was an open-label, multicenter, non-randomized phase 1 study evaluating the PK and pharmacodynamics (PD) of a self-administered vaginal ring system. Enrolled women were healthy, aged 18-38 years, who had regular cycles of 28±7 days and were sexually active with no intention of becoming pregnant for the following 13 months. Exclusion criteria were those typical for oral contraceptive assessments. Women were not permitted to use hormonal contraceptives (other than the study vaginal ring) or vaginal lubricants that could have interfered with steroid absorption during the study.

Women were instructed to use the SA/EE vaginal ring system for 13 consecutive cycles following a 21-days-in and 7-days-out regimen, with the initial ring insertion (day 1 of cycle 1) under investigator supervision at the study clinic. Exposure to SA and EE was 273 days (13 cycles of 21 consecutive days of ring use) for women who completed all 13 cycles. In the PK substudy, blood samples from each woman were collected 8 times (see below) per assessed cycles (1, 3, and 13) as per the protocol.

### Determination of Serum Concentrations for SA and EE at Multiple Time Points

For serum SA and EE measurements, blood samples were collected on treatment days 1-5, 8, 15, and 22 during cycles 1, 3, and 13 with the serum stored at −20° C. Steroid concentrations were determined at each time point using a validated gas chromatography/tandem mass spectrometry (GC-MS/MS; Taylor Technology, Princeton, NJ); the lower limits of quantification (LLOQ) were 50 pg/mL (135 pmol/L) for SA and 10 pg/mL (33.74 pmol/L) for EE. The linear calibration range was 50.0 to 5000 pg/mL for SA and 10.0 to 1000 pg/mL for EE. The inter-assay coefficient of variation for SA was 7-15.5% and for EE 8-13% for low, medium, and high-quality control samples used during assays. The intra-assay coefficient of variation for both SA and EE was lower than 12%.

### Regression Model of Continuous Vaginal Ring Use

A regression model was constructed post hoc to describe the relationship between serum SA concentration and the number of days of vaginal ring use and to extrapolate the expected SA concentration after one year of continuous use. Serum SA data points taken in the first 72 hours following vaginal ring insertion and from days 1-3 from each cycle were not included, as the initial burst of steroid release from insertion/reinsertion would not be predictive of serum SA levels with continuous use. Serum SA levels measured after vaginal ring removal were also excluded. Data from subjects who were not adherent during vaginal ring use were also excluded. Adherence was evaluated in the primary PK sub-study, which was based on diary recordings and observed progesterone/estradiol levels. Serum measurements were log transformed for regression modeling. Serum SA measurements were from cycles 1, 3 and 13. The day of use (if continuous) was calculated as (cycle −1)*21+cycle-day of use (21/7). The last measurement was from cycle 13, day 15, corresponding to the 267th day of use. Using the model's parameters, serum SA levels after 364 days of hypothetical use and the 95% confidence interval (95% CI) around the predicted values were predicted as well as the mean. A serum SA level above 100 pmol/L has previously been shown to suppress ovulation. A similar modeling approach was used for EE.

Linear mixed models adjusting for the repeated measurements from each subject during the study were constructed. Linear regression models without a repeated measure adjustment were also constructed for comparison and simplicity. The models were fitted using SAS version 9.4 (SAS Institute, Cary, NC).

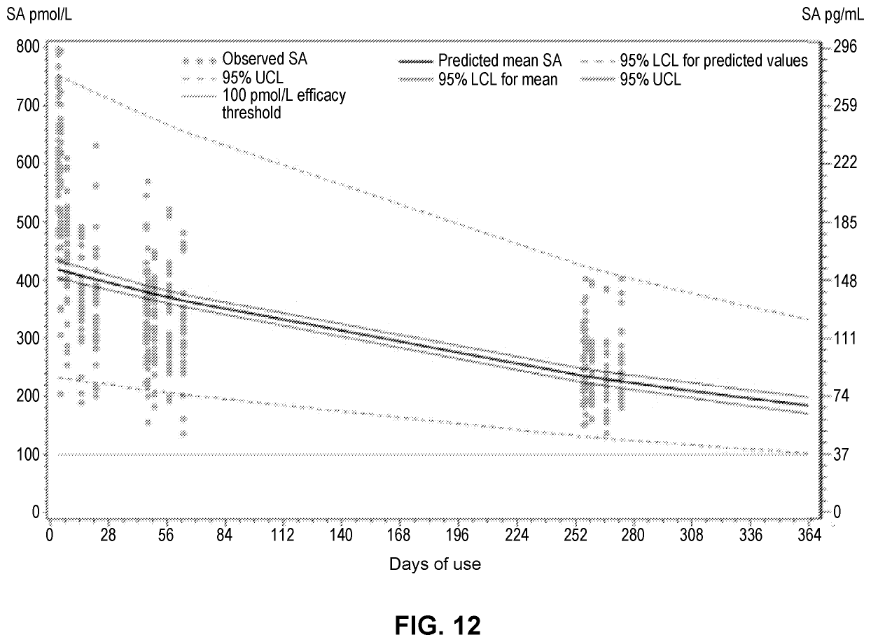

FIG. 12 shows the observed SA values, the predicted regression line along with the 95% CI around the predicted values, and the mean for up to 364 days of use. Based on the results of the linear regression model, the mean serum SA level after 364 days of continuous vaginal ring use was predicted to be 184 pmol/L (68 ng/mL) with a 95% CI of 171-198 pmol/L for the predicted mean. The 95% CI for the predicted values ranged between 102 and 332 pmol/L, meaning that 95% of the serum measurements on day 364 are expected to be between 102 and 332 pmol/L, which are higher than the minimal levels needed for efficacy (i.e., SA levels >100 pmol/L).

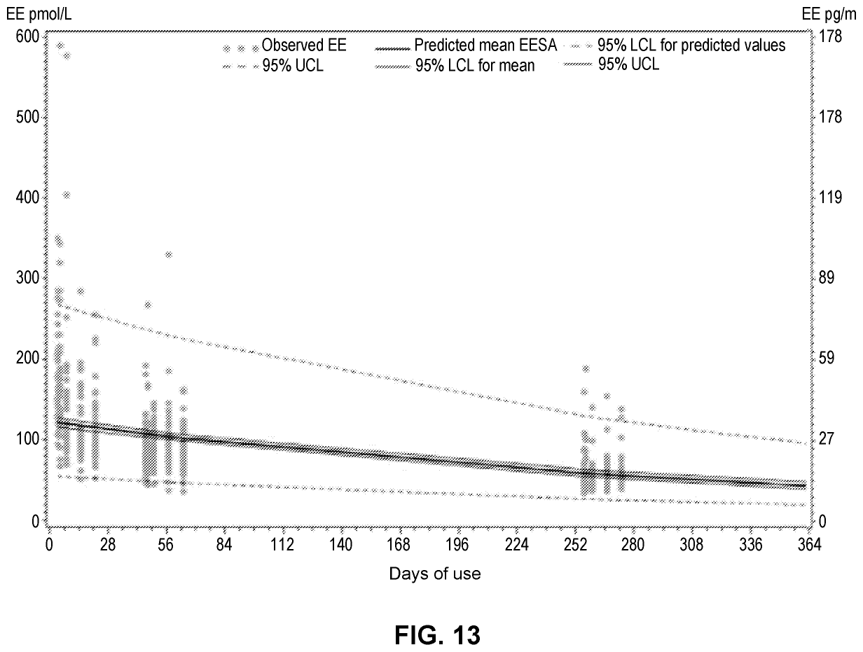

FIG. 13 shows the observed EE values, the predicted regression line along with the 95% CI around the predicted values, and the mean for up to 364 days of use. Based on the results of the linear regression model, the mean serum EE level after 364 days of continuous vaginal ring use was predicted to be 43 pmol/L (12.7 ng/mL) with a 95% CI of 39-48 pmol/L for the predicted mean. The 95% CI for the predicted values ranged between 19 and 95 pmol/L, meaning that 95% of the serum measurements on day 364 are expected to be between 19 and 95 pmol/L.

The regression model supports the use of the vaginal ring system in a flexible extended (i.e., quarterly) or continuous regimen for up to 364 days based on an estimated mean serum SA level of 184 pmol/L with a 95% CI lower limit of 102 pmol/L, which is higher than the 100 pmol/L SA previously shown to suppress ovulation.

The risk of developing a venous thromboembolism event (VTE) has been shown to be dependent on the estrogen dose, with an increased VTE risk with higher estrogen doses. The continuous use model using linear regression surprisingly predicted no accumulation of serum EE levels at 364 days, by showing a steady decline in EE levels over time with cyclical use; alleviating some of the concerns regarding VTE risk with continuous vaginal ring use. In contrast, higher EE levels have been observed with the continuous use of transdermal patches (norelgestromin/EE 150/35 [mcg/mcg] per day) for 12 weeks (˜40 pg/mL) with estimated increase of 2.15 pg/mL EE per week). Higher levels of EE (50.4 pg/mL) were also observed after one cycle (3 weeks) of a patch releasing a lower amount of EE (norelgestromin/EE 150/20 [mcg/mcg] per day).

Results from the predictive modeling support a safe, one-year continuous use of the ring system without removal and reinsertion.

### Example 13: Segesterone Acetate and Ethinyl Estradiol Levels with a Regression Model of Quarterly Use

The regression model described in Example 12 was used to predict AUC and C_{avg} values for SA and EE after four quarterly periods of extended use of the vaginal system disclosed herein. Table 10 shows the pharmacokinetic values for segesterone acetate predicted by the model described herein over four 84-day product use periods, with each of the first three product-use periods followed by a 7-day removal period. C_{avg} is the SA concentration in serum during the product-use period while the AUC_{F-L} is the area under the curve for the period beginning on the first day (“F”) and ending on the last day (“L”) of the product-use cycle.

TABLE 10
84-Day Quarterly Product-Use Period Segesterone
Acetate Predicted C_{avg} and AUC Values

| Quarterly Product- | C_{avg} (pmol/L) | AUC_{F-L}(pmol*h/L) |
|---|---|---|
| Use Period | (95% CI) | (95% CI) |
| 1 | 383 (378- | 776,463 (753,054- |
| | 387) | 800,606) |
| 2 | 317 (313- | 638,853 (620,577- |
| | 320) | 657,671) |
| 3 | 261 (258- | 527,507 (506,390- |
| | 264) | 549,516) |
| 4 | 217 (214- | 463,366 (437,087- |
| | 219) | 491,239) |

Table 11 shows the pharmacokinetic values for segesterone acetate predicted by the model described herein over four quarterly 88-day product use periods, wherein each of the first three product-use cycles are followed by a removal period of 3 days. C_{avg} is the SA concentration in serum during the product-use period while the AUC_{F-L} is the area under the curve for the period beginning on the first day and ending on the last day of the product-use cycle.

TABLE 11
88-Day Quarterly Product-Use Period Segesterone
Acetate Calculated PK Values

| Product- | C_{avg} (pmol/L) | AUC_{F-L}(pmol*h/L) |
|---|---|---|
| Use Period | (95% CI) | (95% CI) |
| 1 | 381 (377- | 809,739 (785,437- |
| | 386) | 834,798) |
| 2 | 312 (308- | 660,280 (641,059- |
| | 316) | 680,082) |
| 3 | 255 (252- | 557,123 (533,659- |
| | 258) | 581,635) |
| 4 | 209 (207- | 452,964 (425,816- |
| | 212) | 481,860) |

As shown in Tables 10 and 11, the average SA concentration in serum during each product-use period is over the minimal levels needed for efficacy (i.e., SA levels >100 pmol/L) indicating that the vaginal ring described herein can be effectively used to prevent pregnancy over four quarterly product-use periods.

Table 12 shows the ethinyl estradiol values calculated over four 84-day quarterly product use periods, wherein each of the first three product-use cycles is followed by a removal period of 7 days. C_{avg} is the average concentration in serum during the product-use period while the AUC_{F-L} is the area under the curve for the period beginning on the first day and ending on the last day of the product-use cycle.

TABLE 12
84-Day Quarterly Product-Use Period Ethinyl
Estradiol Calculated PK Values

| Quarterly Product- | C_{avg} (pmol/L) | AUC_{F-L}(pmol*h/L) |
|---|---|---|
| Use Period | (95% CI) | (95% CI) |
| 1 | 109 (107- | 210,261 (201,764- |
| | 110) | 219,118) |
| 2 | 85 (84- | 172,431 (165,641- |
| | 87) | 179,501) |
| 3 | 67 (66- | 135,316 (127,696- |
| | 68) | 143,396) |
| 4 | 53 (52- | 99,279 (91,246- |
| | 53) | 108,024) |

Table 13 shows the ethinyl estradiol values calculated over four 88-day quarterly product use periods, wherein each of the first three product-use cycles is followed by a removal period of 3 days. C_{avg} is the average EE concentration in serum during the product-use period while the AUC_{F-L} is the area under the curve for the period beginning on the first day and ending on the last day of the product-use cycle.

TABLE 13
88-Day Quarterly Product-Use Period Ethinyl
Estradiol Calculated PK Values

| Quarterly Product- | Cavg (pmol/L) | AUC_{F-L}(pmol*h/L) |
|---|---|---|
| Use Period | (95% CI) | (95% CI) |
| 1 | 108 (106- | 224,510 (215,488- |
| | 110) | 233,913) |
| 2 | 84 (83- | 177,584 (170,450- |
| | 85) | 185,018) |
| 3 | 95 (64- | 137,760 (129,641- |
| | 66) | 146,395) |
| 4 | 51 (50- | 106,867 (97,841- |
| | 51) | 116,733) |

As with the 364-day continuous-use model, the quarterly-use model surprisingly predict no accumulation of serum EE levels at the end of the fourth quarterly product-use period in either the 84-day or 88-day models, instead showing a steady decline in EE levels over time with cyclical use. These data support the safe use of the vaginal ring for quarterly use.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The breadth and scope of the present disclosure should not be limited by any of the above-described exemplary embodiments but should be defined only in accordance with the following claims and their equivalents.

What is claimed is:

1. A method of preventing pregnancy in a female of reproductive potential over a consecutive 364-day product-use period, the method comprising:
(a) inserting into the vagina of the female a vaginal ring system, the ring system comprising:
i. a silicone elastomer ring body having a platinum concentration of approximately 3 ppm to approximately 10 ppm and a hydride/vinyl ratio from approximately 1:1 to approximately 1.3:1 before curing; and
a. a first cylindrical channel adapted to receive a first cylindrical core; and
b. a second cylindrical channel adapted to receive a second cylindrical core;
ii. a first cylindrical core disposed within the first cylindrical channel, wherein the first cylindrical core comprises first and second condensation-cure silicone elastomers, dibutyltin dilaurate, and a viscosity agent selected from the group consisting of diatomaceous earth, cellulose, talc, and silica, and wherein the first cylindrical core comprises approximately 50% segesterone acetate by mass;
iii. a second cylindrical core disposed within the second cylindrical channel, the second cylindrical core comprising a third condensation-cure silicone elastomer, wherein the second and third condensation-cure silicone elastomers are optionally the same, and dibutyltin dilaurate, and wherein the second cylindrical core comprises approximately 40% segesterone acetate by mass and approximately 12% ethinyl estradiol by mass; and
iv. approximately 103 mg of segesterone acetate and approximately 17.4 mg of ethinyl estradiol, wherein both the segesterone acetate and ethinyl estradiol are contained within the cores of the vaginal ring system;
wherein:
the first and second cylindrical cores have a volume ratio of about 11:18; and
(b) retaining the ring system in the vagina for 364 consecutive days,
wherein the method achieves in a serum sample of a female subject one or more of the following:
i) a segesterone acetate concentration in serum on day 364 of approximately 100 pmol/L to approximately 350 pmol/L; and
ii) an ethinyl estradiol concentration in serum on day 364 of approximately 10 pmol/L to approximately 100 pmol/L.

2. The method of claim 1, wherein the method achieves in a serum sample of a female subject one or both of:
i) a segesterone acetate concentration in serum on day 364 of approximately 184 pmol/L on average; and
ii) an ethinyl estradiol concentration in serum on day 364 of approximately 43 pmol/L on average.

3. The method of claim 1 or 2, wherein the method further achieves in a serum sample of a female subject one or both of the following during the 364-day product-use period:
iii) a segesterone acetate Cₘₐₓ of approximately 3,097±850 pmol/L of segesterone acetate over the 364-day product-use period; and
iv) an ethinyl estradiol Cₘₐₓ of approximately 435±131 pmol/L of ethinyl estradiol over the 364-day product-use period.

4. A method of preventing pregnancy in a female of reproductive potential, the method comprising:
(a) initially inserting into the vagina of the female a reusable vaginal ring system; and
(b) removing the vaginal ring system on the day following the end of a consecutive 80- to 90-day product-use period;
wherein the vaginal ring system comprises:
i. a silicone elastomer ring body having a platinum concentration of approximately 3 ppm to approximately 10 ppm and a hydride/vinyl ratio from approximately 1:1 to approximately 1.3:1 before curing; and
a. a first cylindrical channel adapted to receive a first cylindrical core; and
b. a second cylindrical channel adapted to receive a second cylindrical core;
ii. a first cylindrical core disposed within the first cylindrical channel, wherein the first cylindrical core comprises first and second condensation-cure silicone elastomers, dibutyltin dilaurate, and a viscosity agent selected from the group consisting of diatomaceous earth, cellulose, talc, and silica, and wherein the first cylindrical core comprises approximately 50% segesterone acetate by mass;
iii. a second cylindrical core disposed within the second cylindrical channel, the second cylindrical core comprising a third condensation-cure silicone elastomer, wherein the second and third condensation-cure silicone elastomers are optionally the same, and dibutyltin dilaurate, and wherein the second cylindrical core comprises approximately 40% segesterone acetate by mass and approximately 12% ethinyl estradiol by mass; and
iv. approximately 103 mg of segesterone acetate and approximately 17.4 mg of ethinyl estradiol, wherein both the segesterone acetate and ethinyl estradiol are contained within the cores of the vaginal ring system;
wherein:
the first and second cylindrical cores have a volume ratio of approximately 11:18;
wherein the method achieves in a serum sample of a female subject one or both of the following:
i) an average segesterone acetate concentration in serum during the product-use period of approximately 350 pmol/L to approximately 425 pmol/L; and
ii) an average ethinyl estradiol concentration in serum during the product-use period of approximately 75 pmol/L to approximately 125 pmol/L.

5. The method of claim 4, further comprising:
(c) storing the removed vaginal ring system for a removal period of approximately 3 to 7 days including the removal date of step (b), wherein the product-use period and removal period together comprise a product-use cycle; and
(d) repeating steps (a), (b), and (c), for a total of up to four product-use cycles.

6. The method of claim 5, wherein the method achieves in a serum sample of a female subject one or more of the following:
i) an average segesterone acetate concentration in serum during the second quarterly product-use period of approximately 275 pmol/L to approximately 350 pmol/L;
ii) an average segesterone acetate concentration in serum during the third quarterly product-use period of approximately 225 to approximately 300 pmol/L of a third quarterly product-use period;
iii) an average segesterone acetate concentration in serum during the fourth quarterly product-use period of approximately 175 to approximately 250 pmol/L;
iv) an average ethinyl estradiol concentration in serum during the second quarterly product-use period of approximately 75 pmol/L to approximately 100 pmol/L;
v) an average ethinyl estradiol concentration in serum during the third quarterly product-use period of approximately 55 to approximately 75 pmol/L; and
vi) an average ethinyl estradiol concentration in serum during the fourth quarterly product-use period approximately 40 to approximately 60 pmol/L.

7. The method of claim 4, wherein the method further achieves in a serum sample of a female subject one or both of the following:
iii) an average segesterone acetate AUC_{1F-1L} of approximately 725,000 pmol*h/L to approximately 825,000 pmol*h/L of a first product-use period; and
iv) an average ethinyl estradiol AUC_{1F-1L} of approximately 190,000 pmol*h/L to approximately 240,000 pmol*h/L of a first product-use period.

8. The method of claim 5 or 6 wherein the method further achieves in a serum sample of a female subject one or more of the following:
i) an average segesterone acetate AUC_{2F-2L} of approximately 600,000 pmol*h/L to approximately 675,000 pmol*h/L;
ii) an average segesterone acetate AUC_{3F-3L} of approximately 480,000 pmol*h/L to approximately 575,000 pmol*h/L;
iii) a segesterone acetate AUC_{4F-4L} of approximately 400,000 pmol*h/L to approximately 510,000 pmol*h/L;
iv) an average ethinyl estradiol AUC_{2F-2L} of approximately 150,000 pmol*h/L to approximately 200,000 pmol*h/L;
v) an average ethinyl estradiol AUC_{3F-3L} of approximately 100,000 pmol*h/L to approximately 150,000 pmol*h/L; and
vi) an average ethinyl estradiol AUC_{4F-4L} of approximately 75,000 pmol*h/L to approximately 125,000 pmol*h/L.

9. The method of any one of claims 4 to 8, wherein the method further achieves in a serum sample of a female subject one or both of the following:
i) a segesterone acetate Cₘₐₓ of approximately 3,097±850 pmol/L of segesterone acetate over the first product-use period; and
ii) an ethinyl estradiol Cₘₐₓ of approximately 435±131 pmol/L of ethinyl estradiol over the first product-use period.

10. The method of any one of claims 4 to 9, wherein the product-use period consists of 80 consecutive days.

11. The method of any one of claims 4 to 9, wherein the product-use period consists of 81 consecutive days.

12. The method of any one of claims 4 to 9, wherein the product-use period consists of 82 consecutive days.

13. The method of any one of claims 4 to 9, wherein the product-use period consists of 83 consecutive days.

14. The method of any one of claims 4 to 9, wherein the product-use period consists of 84 consecutive days.

15. The method of any one of claims 4 to 9, wherein the product-use period consists of 85 consecutive days.

16. The method of any one of claims 4 to 9, wherein the product-use period consists of 86 consecutive days.

17. The method of any one of claims 4 to 9, wherein the product-use period consists of 87 consecutive days.

18. The method of any one of claims 4 to 9, wherein the product-use period consists of 88 consecutive days.

19. The method of any one of claims 4 to 9, wherein the product-use period consists of 89 consecutive days.

20. The method of any one of claims 4 to 9, wherein the product-use period consists of 90 consecutive days.

21. The method of claim 5, wherein the removal period consists of 3 consecutive days.

22. The method of claim 5, wherein removal period consists of 4 consecutive days.

23. The method of claim 5, wherein removal period consists of 5 consecutive days.

24. The method of claim 5, wherein removal period consists of 6 consecutive days.

25. The method of claim 5, wherein removal period consists of 7 consecutive days.

26. The method any one of claims 1 to 25, wherein approximately 80% to approximately 90% of the ethinyl estradiol is recoverable from the ring system after approximately 18 months of storage at 25° C. and 60% relative humidity and wherein no more than approximately 10% to approximately 20% of the ethinyl estradiol undergoes hydrosilylation with unreacted hydrosilane in the ring body after approximately 18 months of storage at 25° C. and 60% relative humidity.

27. The method of any one of claims 1 to 26, wherein the first cylindrical core has a length of approximately 11 mm.

28. The method of any one of claims 1 to 27, wherein the second cylindrical core has a length of approximately 18 mm.

29. The method of any one of claims 1 to 28, wherein the first cylindrical core has a diameter of approximately 3 mm.

30. The method of any one of claims 1 to 29, wherein the second cylindrical core has a diameter of approximately 3 mm.

31. The method of any one of claims 1 to 30, wherein the first and second cylindrical channels each have a diameter of approximately 3 mm.

32. The method of any one of claims 1 to 31, wherein the first and second cylindrical cores are secured in the first and second channels, respectively, with an adhesive.

33. The method of any one of claims 1 to 32, wherein the first cylindrical core is substantially longitudinally centered in the first cylindrical channel, further wherein the second cylindrical core is substantially longitudinally centered within the second cylindrical channel.

34. The method of any one of claims 1 to 33, wherein
a. the first cylindrical core has a first end face and a second end face, wherein the first cylindrical core is fully disposed within the first cylindrical channel;
b. the second cylindrical core has a first end face and a second end face, wherein the second cylindrical core is fully disposed within the second cylindrical channel; and
c. an end face of the first cylindrical core is substantially coplanar with an end face of the second cylindrical core.

35. The method of any one of claims 1 to 34, wherein the first cylindrical channel and the second cylindrical channels each have lengths of approximately 27 mm.

36. The method of any one of claims 1 to 35, wherein the first and second cylindrical channels are substantially parallel to each other.

37. The method of any one of claims 1 to 36, wherein any void spaces in the first and second cylindrical channels not occupied by the first and second cylindrical cores are filled with adhesive.

38. The method of any one of claims 1 to 37, wherein the ring body has an outer diameter, an inner diameter, and a cross-sectional diameter.

39. The method of claim 38, wherein the outer diameter is approximately 56 mm.

40. The method of claim 35 or 39, wherein the inner diameter is approximately 40 mm.

41. The method of any one of claims 35 to 40, wherein the cross-sectional diameter is approximately 8.4 mm.

42. The method of any one of claims 1 to 41, wherein the silicone elastomer ring body has a platinum concentration of approximately 4 ppm to approximately 9 ppm before curing.

43. The method of claim 42, wherein the silicone elastomer ring body has a platinum concentration of approximately 5 ppm to approximately 8 ppm before curing.

44. The method of any one of claims 1 to 43, wherein the silicone elastomer ring body has a shore A hardness of approximately 25 to approximately 30, a mean fatigue parallel to the cores of approximately 95% and a mean fatigue perpendicular to the cores of approximately 98%.

45. The method of any one of claims 1 to 44, wherein the first cylindrical core is impregnated with a first amount of segesterone acetate particles having a particle size distribution: D90 of not more than 10 microns and a D50 of not more than 5 microns; further wherein in the second cylindrical core is impregnated with a second amount of segesterone acetate particles and an amount of ethinyl estradiol particles, wherein the ethinyl estradiol particles have a particle size distribution of 100% max 15 microns, 99% max 12.5 microns, 95% max 10 microns and max 40% less than or equal to 1.3 microns.

46. The method of any one of claims 1 to 45, wherein at least 75% of the segesterone acetate comprises segesterone acetate Polymorphic form I.

47. The method of any one of claims 1 to 46, wherein the segesterone acetate comprises up to 25% segesterone acetate Polymorphic form II.

48. The method of any one of claims 1 to 47, wherein after 18 months of storage of the vaginal system, at least one degradation product selected from the group consisting of 6α-OH-EE, 6β-OH-EE, 6α-OH-NES, 6β-OH-NES, 17β-estradiol, NES ST-alcohol, NES iso-ST-alcohol, 6,7-didehydro-EE & 9,11-didehydro-EE, estrone, Δ⁶-NES, Iso-NES, 3-enolacetate-NES, 3-methoxy-NES, and combinations thereof, is detectable but does not account for more than 5% of ring extractables as measured by HPLC.

49. The method of claim 48, wherein the at least one degradation product is detectable but does not account for more than 1% of ring extractables as measured by HPLC.

50. The method of any one of claims 1 to 49, wherein the ring body comprises approximately 4% TiO₂ by weight.
